(19) United States
(12) Patent Application Publication
(10) Pub. No.: US 2015/0018384 A1
(43) Pub. Date: Jan. 15, 2015

(54) TREATMENT FOR HIGH CHOLESTEROL

(71) Applicant: FibroGen, Inc., San Francisco, CA (US)

(72) Inventors: Al Y Lin, Castro Valley, CA (US); Thomas B. Neff, Atherton, CA (US); Kin-Hung Yu, Hillsborough, CA (US)

(73) Assignee: FibroGen, Inc., San Francisco, CA (US)

(21) Appl. No.: 14/381,955

(22) PCT Filed: Mar. 8, 2013
(86) PCT No.: PCT/US2013/029851

§ 371 Date: Aug. 28, 2014

Related U.S. Application Data
(60) Provisional application No. 61/609,007, filed on Mar. 9, 2012.
(60) Provisional application No. 61/650,043, filed on May 22, 2012.

(51) Int. Cl. C07D 471/04; C07D 217/26

(52) U.S. Cl. CPC C07D 471/04; C07D 217/26
USPC 514/300; 514/309

(57) ABSTRACT

The present invention provides a method for treatment of high cholesterol by reducing low density lipoprotein cholesterol (LDL-C) and/or very low density Opoproiein cholesterol (VLDL-C) in subjects In need thereof by administering a compound that inhibits HIF hydroxylase activity. The method is useful, for reducing LDL cholesterol levels and total cholesterol levels even In subjects already undergoing treatment with other cholesterol-lowering medications, for example statins, fibrates, nicotinic acids and bile acid-binding resins, and in patients having chronic kidney disease or end stage renal disease, inter alia.

### CROSS REFERENCE TO RELATED APPLICATIONS

This application claims the benefit under 35 U.S.C. §119(e) of U.S. Provisional Application 61/609,007, filed Mar. 9, 2012, and U.S. Provisional Application 61/650,043, filed May 22, 2012, which applications are hereby incorporated by reference in their entirety.

### FIELD OF THE INVENTION

The invention relates to treatment methods for reducing the circulating level of total cholesterol and particularly for reducing the circulating level of LDL-C and/or VLDL-C, and/or increasing the ratio of HDL-C/LDL-C.

### BACKGROUND OF THE INVENTION

In the past 25 years, a large body of evidence from numerous clinical trials has demonstrated that pharmacological reagents that reduce total blood cholesterol, and particularly low density lipoprotein-cholesterol (LDL-C) levels, also decrease the risk of coronary heart disease (CHD). The totality of the trial data to date supports the idea that lowering the circulating LDL-C levels results directly in reduction of coronary events (including fatal and non-fatal heart attacks) and strongly suggests that the lowering of LDL-C should be the principal therapeutic goal in any lipid-altering therapy. (See, National Cholesterol Education Program Guidelines, NHLBI/NIH Publication May 2001; Grundy et al. (2004) Circulation 110:227.) A number of agents are currently available for lowering total cholesterol, including HMGCoA reductase inhibitors (e.g., simvastatin, lovastatin, pravastatin, fluvastatin, atorvastatin, etc.), various forms of nicotinic acid, fibric acids, and bile acid-binding resins. These agents, although effective in many patients, often have undesirable side effects. Statins can cause myopathy and increased liver enzymes; nicotinic acid agents can cause flushing, hyperglycemia, hyperuricemia, hepatotoxicity, and gastrointestinal distress; fibric acids can cause dyspepsia, gallstones, and myopathy; bile acid-binding resins can cause GI distress, constipation, and decreased absorption of other drugs. Therefore, a need remains for additional cholesterol-lowering agents with fewer side effects. In addition, for some patients, currently available agents do not provide a sufficient level of reduction of total cholesterol and/or LDL-C. Thus, additional cholesterol lowering agents that are more effective or that can be used in combination with current therapies to achieve greater reductions in total cholesterol or LDL-C levels would be desirable.

### SUMMARY OF THE INVENTION

The present invention relates to methods for treatment of high cholesterol by reducing the circulating level of total cholesterol and particularly by reducing the circulating level of LDL-C and/or VLDL-C, and/or increasing the ratio of HDL-C/LDL-C.

In particular aspects, the present invention provides methods of reducing the circulating level of low density lipoprotein cholesterol (LDL-C) and/or reducing the circulating level of very low density lipoprotein cholesterol (VLDL-C) in subjects in need of such therapy. In other aspects, the invention also provides a method of reducing the circulating levels of both VLDL-C and LDL-C, and a method of reducing the levels of both LDL-C and serum triglycerides. In addition, the invention provides a method of reducing LDL-C levels in subjects undergoing treatment with other cholesterol lowering drugs but for whom the circulating level of LDL-C remains undesirably high. These and other methods as described herein are accomplished by administering to the subject an effective amount of a compound that inhibits HIP hydroxylase activity. Particular aspects of the invention are described herein.

Also provided is the use of a compound that inhibits HIF hydroxylase activity in the manufacture of a medicament for treating high cholesterol, for reducing the circulating level of LDL-C and/or reducing the circulating level of very low density lipoprotein cholesterol (VLDL-C). Also provided is a compound that inhibits HIP hydroxylase activity for use in treating high cholesterol, for use in reducing the circulating level of LDL-C and/or for use in reducing the circulating level of very low density lipoprotein cholesterol (VLDL-C).

In one embodiment, the invention provides a method of reducing the circulating level of low density lipoprotein cholesterol (LDL-C) in a subject in need thereof, the method comprising administering to the subject an effective amount of a compound that inhibits HIF hydroxylase activity, whereby the circulating level of LDL-C in said subject is reduced.

In another embodiment, the invention provides method of reducing the circulating level of very low density lipoprotein cholesterol (VLDL-C) in a subject in need thereof, the method comprising administering to the subject an effective amount of a compound that inhibits HIF hydroxylase activity, whereby the circulating level of VLDL-C in said subject is reduced.

In a further embodiment, the invention provides a method of reducing the circulating levels of very low density lipoprotein cholesterol (VLDL-C) and low density lipoprotein cholesterol (LDL-C) in a subject in need thereof, the method comprising administering to the subject an effective amount of a compound that inhibits HIF hydroxylase activity, whereby the circulating levels of both LDL-C and VLDL-C in said subject are reduced.

In another embodiment, the invention provides a method of reducing the circulating level of total cholesterol in a subject under treatment for high cholesterol with a separate cholesterol-lowering agent, the method comprising administering to the subject an effective amount of a compound that inhibits HIF hydroxylase activity, whereby the circulating level of total cholesterol is reduced. In some embodiments, the separate cholesterol-lowering agent is selected from the group consisting of a HMGCoA reductase inhibitor, a nicotinic acid, a fibric acid, and a bile acid-binding resin.

In another embodiment, the invention provides a method of reducing the circulating levels of LDL-C and/or VLDL-C in a subject under treatment for high cholesterol with a cholesterol-lowering agent, the method comprising administering to the subject an effective amount of a compound that inhibits HIF hydroxylase activity, whereby the circulating levels of LDL cholesterol and/or VLDL cholesterol are reduced. In particular embodiments, the cholesterol-lowering agent is selected from the group consisting of a HMGCoA reductase inhibitor, a nicotinic acid, a fibric acid, and a bile acid-binding resin.

In another embodiment, the invention provides a method of increasing the ratio of HDL-C/LDL-C in a subject in need thereof, the method comprising administering to the subject an effective amount of a compound that inhibits HIF hydroxylase activity, whereby the ratio of HDL-C/LDL-C in said subject is increased.

In yet another embodiment, the invention provides a method of decreasing the ratio of LDL-C/HDL-C in a subject in need thereof, the method comprising administering to the subject an effective amount of a compound that inhibits HIF hydroxylase activity, whereby the ratio of LDL-C/HDL-C in said subject is decreased.

In another embodiment, the invention provides a method of decreasing the ratio of total cholesterol/HDL-C in a subject in need thereof, the method comprising administering to the subject an effective amount of a compound that inhibits HIF hydroxylase activity, whereby the ratio of total cholesterol/HDL-C in said subject is decreased.

Other methods of regulating and maintaining cholesterol levels are also provided herein.

Suitable compound is any compound that inhibits HIF hydroxylase activity. In particular embodiments of the above methods, the compound that inhibits HIF hydroxylase activity is a heterocyclic carboxamide. In some embodiments of the above methods, the compound that inhibits HIF hydroxylase activity is a structural mimetic of 2-oxoglutarate. In certain embodiments of the above methods, the compound that inhibits HIF hydroxylase activity is a heterocyclic carbonyl glycine. In some embodiments, the compound that inhibits HIF hydroxylase activity is a compound of Formula I. In other embodiments, the compound that inhibits HIF hydroxylase activity is an isoquinoline carboxamide. In other particular embodiments, the compound that inhibits HIF hydroxylase activity is a compound of Formula II or a compound of Formula III or a compound of Formula IV.

In other embodiments the compound that inhibits HIF hydroxylase activity is selected from the group consisting of [(4-Hydroxy-1-methyl-7-phenoxy-isoquinoline-3-carbonyl)-amino]-acetic acid, [(1-Chloro-4-hydroxy-isoquinoline-3-carbonyl)-amino]-acetic acid, {[5-(4-Chloro-phenoxy)-1-cyano-4-hydroxy-isoquinoline-3-carbonyl]-amino}-acetic acid, {[4-Hydroxy-7-(4-methoxy-phenoxy)-isoquinoline-3-carbonyl]-amino}-acetic acid, [(1-Cyano-4-hydroxy-5-phenoxy-isoquinoline-3-carbonyl)-amino]-acetic acid, {[2,3-Dichloro-7-cyano-4-hydroxy-1-(4-methoxy-benzyl)-1H-pyrrolo[2,3-c]pyridine-5-carbonyl]-amino}-acetic acid, {[7-Cyano-1-(2-fluoro-benzyl)-4-hydroxy-1H-pyrrolo[2,3-c]pyridine-5-carbonyl]-amino}-acetic acid, [(1-Cyano-4-hydroxy-7-isopropoxy-isoquinoline-3-carbonyl)-amino]-acetic acid, {[1-Cyano-7-(2,6-dimethyl-phenoxy)-4-hydroxy-isoquinoline-3-carbonyl]-amino}-acetic acid, [(7-Cyano-4-hydroxy-1-naphthalen-2-ylmethyl-1H-pyrrolo[2,3-c]pyridine-5-carbonyl)-amino]-acetic acid, {[3-Bromo-7-cyano-4-hydroxy-1-(4-methoxy-benzyl)-1H-pyrrolo[2,3-c]pyridine-5-carbonyl]-amino}-acetic acid, [(1-Chloro-4-hydroxy-7-trifluoromethyl-isoquinoline-3-carbonyl)-amino]-acetic acid, [(1-Chloro-4-hydroxy-5-methyl-isoquinoline-3-carbonyl)-amino]-acetic acid, [(7-Bromo-4-hydroxy-isoquinoline-3-carbonyl)-amino]-acetic acid, {[2-(3,4′-Difluoro-biphenyl-4-ylmethyl)-5-hydroxy-6-isopropyl-3-oxo-2,3-dihydro-pyridazine-4-carbonyl]-amino}-acetic acid, [(1-Hydroxy-4,4-dimethyl-3-oxo-3,4-dihydro-naphthalene-2-carbonyl)-amino]-acetic acid, 4-Oxo-1,4-dihydro-[1,10]phenanthroline-3-carboxylic acid, {[5-(3-Chloro-phenyl)-3-hydroxy-pyridine-2-carbonyl]-amino}-acetic acid, and [(7-Fluoro-4-hydroxy-2-oxo-2H-thiochromene-3-carbonyl)-amino]-acetic acid. Other suitable compounds are described herein.

In one embodiment of the methods, the subject in need is a human subject. In particular embodiments, the human subject has a high circulating level of total cholesterol. In some embodiments the subject has a circulating level of total cholesterol of 200 mg/dL or greater, or 240 mg/dL or greater.

In other particular embodiments, the human subject has a high circulating level of LDL cholesterol. In some embodiments the subject has a circulating level of LDL cholesterol of 100 mg/dL or greater, or 130 mg/dL or greater, or 160 mg/dL or greater.

In certain embodiments of the present methods, a suitable subject will have one or more of a high circulating level of total cholesterol, a high circulating level of LDL-cholesterol, and a low circulating level of HDL-cholesterol. In some embodiments of the present methods, the subject is undergoing treatment for high cholesterol with a separate cholesterol-lowering medication.

In certain embodiments, the subject is a subject with chronic kidney disease, a subject with end-stage renal disease, a subject with anemia, a subject on dialysis, a subject having coronary heart disease, a subject with diabetes, a subject having atherosclerosis or clinical forms of atherosclerotic disease (e.g., peripheral artery disease (PAD), abdominal aortic aneurysm, and symptomatic carotid artery disease), a subject having hypertension, a subject having HDL-C below 40 mg/dL, a subject having family history of premature CHD, a subject with serum triglyceride level ≧150 mg/dL, or a subject having history of cigarette smoking.

In one embodiment, the method additional comprises administering to the subject a cholesterol-lowering drug selected from the group consisting of a HMGCoA reductase inhibitor, a nicotinic acid, a fibric acid, and a bile acid-binding resin.

In certain embodiments of the method, the circulating LDL-C level is reduced by at least 10%, at least 20/, at least 30%, at least 40%, or more. In certain embodiments of the method the circulating LDL-C level is reduced by at least 10 mg/dL, at least 20 mg/dL, at least 30 mg/dL, at least ≧40 mg/dL, or more.

The invention also provides compounds that inhibit HIF hydroxylase activity for use in regulating cholesterol metabolism and achieving and/or maintaining cholesterol homeostasis, for use in regulating cholesterol biosynthesis, uptake, processing, storage, transport, clearance, and utilization, for use in altering expression of a cholesterol regulatory factor, for use in reducing the circulating level of low density lipoprotein cholesterol (LDL-C) and/or reducing the circulating level of very low density lipoprotein cholesterol (VLDL-C), for use in reducing the circulating levels of both VLDL-C and LDL-C, for use in reducing the levels of both LDL-C and serum triglycerides, and for use in reducing LDL-C levels in subjects undergoing treatment with other cholesterol lowering drugs but for whom the circulating level of LDL-C remains undesirably high.

These and other embodiments of the subject invention will readily occur to those of skill in the art in light of the disclosure herein, and all such embodiments are specifically contemplated.

### BRIEF DESCRIPTION OF THE DRAWINGS

FIG. 1 shows a graph of the change from the baseline level of total cholesterol over time for CKD patients not on dialysis treated with compound A.

FIG. 2 shows a graph of the change from the baseline level of total cholesterol over time for ESRD patients on dialysis treated with compound A (diamond) or EPO (square).

(A) FIG. 3A shows a graph of the change from the baseline of total cholesterol over time for CKD patients not on dialysis treated with compound A. The patients were divided into those concurrently on separate cholesterol-lowering medication (diamonds) and those not on separate cholesterol-lowering medication (squares). (B) FIG. 3B shows a graph of the change from the baseline level of total cholesterol over time for ESRD patients on dialysis treated with compound A (diamonds, squares) or EPO (triangles, X). Each group was divided into those concurrently on separate cholesterol-lowering medication (squares, X) and those not on separate cholesterol-lowering medication (diamonds, triangles).

FIG. 4 shows a graph of the change from the baseline level of total cholesterol over time for ESRD patients on dialysis treated with compound A or EPO, in which patients were stratified according to a prior medical history of hyperlipidemia or hypercholesterolemia. Diamonds—Compound A treated, no hyperlipidemia, Squares—Compound A treated, with history of hyperlipidemia; Triangles—EPO treated, no hyperlipidemia, X-EPO treated, with history of hyperlipidemia.

FIG. 5 shows the total cholesterol over time for CKD patients not on dialysis treated with compound A. The patients were stratified according to their total cholesterol level at baseline (i.e., pre-treatment) of >200 mg/dL (squares) or ≦200 mg/dL (diamonds).

FIG. 6A shows a graph of the change from the baseline level of total cholesterol over time for ESRD patients on dialysis treated with compound A. The patients were stratified according to their total cholesterol level at baseline (i.e., pre-treatment) of >200 mg/dL (squares) or ≦200 mg/dL (diamonds). FIG. 6 B shows a graph of the mean total cholesterol level over time for the same patients as in FIG. 6A.

FIG. 7 shows the mean total cholesterol levels (diamonds) and the mean LDL-C levels (squares) over time in CKD patients not on dialysis treated with compound A.

FIG. 8A is a bar graph showing the total cholesterol, HDL-C, LDL-C, triglyceride (TOG), and VLDL-C levels at 9 weeks after treatment with compound A in ESRD patients on dialysis compared to the level of each component at baseline (pre-treatment). FIG. 8B is a bar graph showing the HDL/LDL ratio at 9 weeks for the patients in FIG. 8A compared to baseline. Baseline ratio set at 1.0.

FIG. 9 is a bar graph showing the % change from baseline at 24 hours after treatment for total cholesterol, HDL-C, LDL-C, and the LDL/HDL in Sprague-Dawley rats treated with a 60 mg/kg dose of compound A.

FIG. 10 shows the dose dependent decrease in total cholesterol in monkeys (n=5) treated daily with compound A at 1 mg/kg (squares), 10 mg/kg (triangles), 30 mg/kg (X), or untreated (diamonds) at pre-dose, day 28 of treatment, and 30 days after the treatment ended.

FIG. 11 shows the dose dependent decrease in total cholesterol in monkeys (n=5) treated with compound A at 1 mg/kg (squares), 10 mg/kg (triangles), 30 mg/kg (X), 40 mg/kg (filled circles), or untreated (diamonds) at pre-dose, day 28 to day 152 of treatment, and 43 days after the treatment ended.

FIG. 12 shows the dose dependent decrease in total cholesterol in monkeys treated daily with compound D at 1 mg/kg (squares), 10 mg/kg (triangles), 30 mg/kg (X), or untreated (diamonds) at pre-dose, day 28 of treatment, and 33 days after the treatment ended. N=3 for the 1 mg/kg and 10 mg/kg samples, n=5 for the other samples.

FIG. 13 shows the dose dependent decrease in total cholesterol in monkeys (n=5) treated daily with compound C at 1 mg/kg (squares), 10 mg/kg (triangles), 30 mg/kg (X), or untreated (diamonds) at pre-dose, day 14, day 29 of treatment, and 57 days after the treatment ended.

FIG. 14 shows a graph of the mean total cholesterol (mg/dL) over time for CKD patients not on dialysis treated with Compound A or placebo, in which the patients were stratified by their baseline (pre-treatment) total cholesterol level (>200 mg/dL or ≦200 mg/dL). Circles—Compound A treated, ≦200 mg/dL (n=48); Triangles—Placebo, ≦200 mg/dL (n=20); Squares—Compound A treated, >200 mg/dL (n=13); Asterisk—Placebo, >200 mg/dL (n=10). Treatment ended at week 9.

FIG. 15 shows a graph of the mean LDL cholesterol (mg/dL) over time for CKD patients not on dialysis treated with Compound A or placebo, in which the patients were stratified by their baseline (pre-treatment) total cholesterol level (>200 mg/dL or ≦200 mg/dL). Circles—Compound A treated, ≦200 mg/dL (n=48); Triangles—Placebo, ≦200 mg/dL (n=20); Squares—Compound A treated, >200 mg/dL (n=13); Asterisk—Placebo, >200 mg/dL (n=10). Treatment ended at week 9.

FIG. 16 shows a graph of the mean total cholesterol (mg/dL) over time for ESRD patients, previously treated with epoetin alfa (EPO), treated with Compound A for 6 weeks or continued on EPO. The patients were stratified by their baseline (pre-treatment) total cholesterol level (>200 mg/dL or ≦200 mg/dL). Circles—Compound A treated, ≦200 mg/dL (n=60); Triangles—EPO, ≦200 mg/dL (n=20); Squares—Compound A treated, >200 mg/dL (n=14); Asterisk—EPO, >200 mg/dL (n=2). Treatment ended at week 7.

FIG. 17 shows a graph of the mean LDL cholesterol (mg/dL) over time for ESRD patients, previously treated with epoetin alfa (EPO), treated with Compound A for 6 weeks or continued on EPO. The patients were stratified by their baseline (pre-treatment) total cholesterol level (>200 mg/dL or ≦200 mg/dL). Circles—Compound A treated, ≦200 mg/dL (n=60); Triangles—EPO, ≦200 mg/dL (n=20); Squares—Compound A treated, >200 mg/dL (n=14); Asterisk—EPO, >200 mg/dL (n=2). Treatment ended at week 7.

FIG. 18 shows a graph of the % change from baseline for total cholesterol levels for healthy human subjects over the course of weekly administration of 0.15 mg/kg Compound C (n=7) or placebo (n=2). Dosing at days 1, 8, 15, and 22.

FIG. 19 shows a graph of the % change from baseline for LDL cholesterol levels for healthy human subjects over the course of weekly administration of 0.15 mg/kg Compound C (n=7) or placebo (n=2). Dosing at days 1, 8, 15, and 22.

FIG. 20 shows a graph of the % change from baseline for total cholesterol levels for healthy human subjects over the course of weekly administration of 0.25 mg/kg Compound C (n=6) or placebo (n=1). Dosing at days 1, 8, 15, and 22.

FIG. 21 shows a graph of the % change from baseline for LDL cholesterol levels for healthy human subjects over the course of weekly administration of 0.25 mg/kg Compound C (n=6) or placebo (n==). Dosing at days 1, 8, 15, and 22.

FIG. 22 is a plot of total cholesterol levels (mg/dL) in ApoE deficient mice dosed with various PHI compounds (60 mg/kg B, E, D, F, or G), rosuvastatin, or vehicle, three times a week for two weeks (n=10/group). A group of 10 mice was sacrified at the beginning of the study for a baseline total cholesterol measurement. Mean±SEM is shown.

FIG. 23 is a plot of the % change from baseline in total cholesterol levels in DIO mice dosed with various PHI compounds (60 mg/kg B, E, D, F, or G), rosuvastatin, or vehicle, three times a week for two weeks (n=10/group except that n=9 for Compound F). Mean±SEM is shown.

FIG. 24 is a plot of the % change from baseline in total cholesterol levels in DIO mice dosed with various PHI compounds (60 mg/kg A; 20 mg/kg L, M, or N), rosuvastatin (20 mg/kg), or vehicle, three times a week for two weeks (n=10/group). Mean±SEM is shown.

FIG. 25A, 25B, 25C, 25D. ApoE deficient mice (n=8/group) were dosed with Compound A or Compound D at 2, 20, 60, or 100 mg/kg, rosuvastatin, or vehicle only, 3 times a week for 4 weeks. Hemoglobin (g/dL) and total cholesterol was measured at the end of the study. Compound A treated mice showed a significant increase in hemoglobin for the 60 mg/kg and 100 mg/kg doses (FIG. 25A). The Compound A treated mice also showed a significant percent change in total cholesterol from baseline for the 100 mg/kg dose (FIG. 25B). The Compound D treated mice did not show a significant increase in hemoglobin at any of the doses tested (FIG. 25C) but these mice exhibited a significant percent change in total cholesterol from baseline for the 20 mg/kg, 60 mg/kg, and 100 mg/kg doses (FIG. 25D).

### DETAILED DESCRIPTION OF THE INVENTION

Before the present compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items. It must be noted that as used herein and in the appended claims, the singular forms “a,” “an,” and “the” include plural references unless context clearly dictates otherwise.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods in Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer Verlag.

The section headings are used herein for organizational purposes only, and are not to be construed as in any way limiting the subject matter

### DEFINITIONS

The terms “disorders,” “diseases,” and “conditions” are used inclusively herein and refer to any condition deviating from normal.

The terms “treating,” “treatment” and the like, are used herein to mean administering a therapy to a patient in need thereof.

An “effective amount” of a compound is an amount sufficient to bring about the desired result in a treated subject, for example, an amount sufficient to treat congestive heart failure, to improve cardiac function, to prevent or reduce decline in cardiac function, or to reduce cardiac damage. The effective amount can vary depending upon the particular compound, the nature or severity of the condition being treated, the age, weight, etc. of the subject being treated, the route of administration or formulation of the compound, and the dosing regimen, among other things. An effective amount can readily be determined by one skilled in the medical arts.

In all embodiments of the invention in which a “reducing” or “increasing” is included, or in which a component or measurement is indicated to be “reduced” or “increased,” unless otherwise indicated, the reduction or increase is determined with respect to the baseline value of the component or measurement. The baseline (BL) value of any component or measurement is the value of the component or measurement prior to any treatment by the present methods, that is, prior to the administration of any compound, vehicle, or placebo to the subject.

### HIF and HIF Hydroxylase

In response to hypoxia, an increase in glycolysis occurs to compensate for energy loss due to reduced oxidative phosphorylation, and erythropoiesis and angiogenesis are upregulated to achieve more efficient oxygen utilization. Central to this adaptive response is the mechanism that uses oxygen deficiency as a signal to activate the transcription of genes important for these processes. The key to this activation mechanism is the rapid accumulation of hypoxia-inducible factor (HIF)-α under hypoxia. HIF is recognized as the key modulator of the transcriptional response to hypoxic stress. HIF-α subunits undergo oxygen-dependent hydroxylation on specific proline residues. Hydroxylated HIF-α subunits are recognized by the von Hippel-Lindau (VHL) protein of the E3 ubiquitin ligase complex, and are rapidly destroyed via the polyubiquitination/proteasomal degradation pathway. As intracellular O₂ concentration is reduced, non-hydroxylated HIF-α subunits accumulate and form functional transcription factors in the nucleus by heterodimerization with the constitutively expressed HIF-lfisubunit.

HIF prolyl hydroxylases (HPHs), also referred to by several other names, including prolyl hydroxylase domain (PHD) proteins, form an evolutionarily conserved subfamily of dioxygenases that use oxygen and 2-oxoglutarate (2-OG) as co-substrates, and iron and ascorbate as cofactors. Under normal oxygen conditions, HIF-α is hydroxylated by the HIF prolyl hydroxylases at specific proline residues, resulting in its ultimate destruction via the polyubiquitination/proteasomal degradation pathway. HIF prolyl hydroxylases are sensitive to oxygen conditions in the cell and become less active under hypoxic conditions, resulting in an accumulation of HIF-α. The hypoxic condition and resulting stabilization of HIF can be mimicked with inhibitors of the HIF prolyl hydroxylases. Interest in this area has increased substantially in the past decade and many HIF prolyl hydroxylase inhibitor compounds have been described

The term “HIF prolyl hydroxylase,” as used herein, refers to an enzyme that is capable of hydroxylating a proline residue within an alpha subunit of hypoxia inducible factor (HIF). Prolyl hydroxylation of HIF is accomplished by a family of proteins variously termed the prolyl hydroxylase domain proteins (PHD1, 2, and 3), also known as HIF prolyl hydroxylases (HPH-3, 2, and 1) or EGLN-2, 1, and 3. The term “HIF hydroxylase” is used herein synonomously with “HIF prolyl hydroxylase.” Such HIP prolyl hydroxylases include protein members of the EGL-9 (EGLN) 2-oxoglutarate- and iron-dependent dioxygenase family described by Taylor (2001) Gene 275:125-132; and characterized by Aravind and Koonin (2001) Genome Biol 2:RESEARCH0007; Epstein et al. (2001) Cell 107:43-54; and Bruick and McKnight (2001) Science 294:1337-1340. (See reviews of the HIF and PHD systems in Majmundar et al. (2010) Mol. Cell. 40.294; Fong and Takeda (2008) Cell Death and Differentiation 15:635; Smith et al. (2008) Brit. J. Haemotol. 141:325).

In the methods of the present invention, reference to a compound that inhibits HIF hydroxylase activity means a compound that inhibits the hydroxylase activity of one or more HIF hydroxylase enzymes. Typically, the compound inhibits the activity of two or three HIF hydroxylase enzymes.

### Lipoproteins

Lipoproteins are macromolecular complexes that transport hydrophobic plasma lipids, particularly cholesterol and triglyceride, in the plasma. More than half of the coronary artery disease in the U.S. is attributable to abnormalities in the levels and metabolism of the plasma lipids and the lipoproteins.

Lipoproteins are spherical particles made up of hundreds of lipid and protein molecules. The major lipids of the lipoprotein particles are cholesterol, triglycerides, and phospholipids. The core of the lipoproteins is composed of nonpolar lipids, primarily triglycerides and the esterified form of cholesterol (cholesteryl esters). Phospholipids and a small amount of non-esterified cholesterol, which are soluble in both aqueous and lipid environments, cover the surface of the particles, where they act as the interface between the plasma the core components.

Lipoproteins have been classified into 5 major groups based on their densities: (1) chylomicrons, (2) very low density lipoproteins (VLDL); (3) intermediate density lipoproteins (IDL) (4) low density lipoproteins (LDL); and (5) high density lipoproteins (HDL). LDL-C (the so-called “bad” cholesterol) is the cholesterol in LDL particles, HDL-C (the so-called “good” cholesterol) is the cholesterol in the HDL particles, VLDL-C is the cholesterol in the VLDL particles, etc.

Cholesterol is an essential component for animal life and serves several major cellular functions including the structural integrity of cell membranes, intracellular transport, cell signaling and nerve conduction, and is used as the precursor molecule for biosynthesis of vitamin D, certain steroid hormones including cortisol and aldosterone, and the sex hormones progesterone, estrogens, testostersone, and their derivatives. However, high levels of cholesterol in the blood, depending upon how it is transported in the lipoproteins, has been associated with the progression of atherosclerosis, leading to myocardial infarction, stroke, peripheral vascular disease, and other disorders. High levels of LDL-C in the blood are strongly correlated with the development of coronary heart disease. Low levels of HDL-C in the blood add further risk of CHD.

The National Cholesterol Education Program (NCEP) Expert Panel on Detection, Evaluation and Treatment of High Blood Cholesterol in Adults has produced a report, endorsed by the National Heart, Lung and Blood Institute of the NIH, the American Heart Association, and the American College of Cardiology Foundation, in which certain guidelines for acceptable blood (i.e., circulating) levels of total cholesterol, LDL-C, HDL-C, VLDL-C and triglycerides were suggested. The acceptable levels for these lipid components vary with the kind and number of risk factors present in the patient. These guidelines have been widely accepted and are generally used for determining the advisability of some type of therapeutic intervention to lower the circulating lipid levels.

In general, the NCEP guidelines suggest that total blood cholesterol below 200 mg/dL (5.2 mmol/L) is desirable; 200-239 mg/dL (5.2-6.2 mmol/L) is borderline high; 240 mg/dL (6.2 mmol/L) and above is high. For purposes of the present invention, a “high circulating level of total cholesterol” in a subject will vary depending upon other existing factors in the subject, and with current guidelines from authoritative medical organizations. Typically, at present, for all patients, a total circulating cholesterol level of 240 mg/dL and higher is a “high circulating level of total cholesterol.” Typically, for patients at higher risk of CHD or with existing CHD, a “high circulating level of total cholesterol” is 200 mg/dL and higher. Thus a “high circulating level of total cholesterol” will vary with the subject to be treated and can be readily determined by competent medical practicioners based on patient history and prevailing guidelines.

For circulating level of HDL-C, the guidelines suggest that 60 mg/dL and above is best; 50-59 mg/dL is good; and below 50 mg/dL (for women) and below 40 mg/dL (for men) is poor (significant CHD risk). Typically, a “low circulating level of HDL-C” is below 50 mg/dL (for women) and below 40 mg/dL (for men).

With respect to the guidelines for circulating level of LDL-C, the recommendations vary depending on the risk status of the patient. According to the guidelines, for patients at high risk of heart disease, circulating level of LDL-C below 70 mg/dL is ideal; for patients at risk of heart disease, circulating level of LDL-C below 100 mg/dL is ideal; for patients with low risk of heart disease, circulating level of LDL-C of 100-129 mg/dL is near ideal; circulating level of LDL-C of 130-159 mg/dL for all patients is borderline high; 160-189 mg/dL is high; and 190 mg/dL and above is very high.

For patients with existing CHD or CHD risk equivalents (which include other clinical forms of atherosclerotic disease (e.g., PAD, abdominal aortic aneurysm, and symptomatic carotid artery disease), and diabetes) a high level of circulating LDL-C is considered to be 100 mg/dL or more. For patients with 2 or more risk factors for CHD (risk factors include cigarette smoking, hypertension, HDL-C below 40 mg/dL, family history of premature CHD, and age (45 and over for men; 55 and over for women), a high level of circulating LDL-C is considered to be 130 mg/dL or more; for patients for 0-1 risk factor for CHD, a high level of circulating LDL-C is considered to be 160 mg/dL or more. Thus a “high circulating level of LDL-C” will vary with the subject to be treated and can be readily determined by competent medical practicioners based on patient history and prevailing guidelines.

Although the recommendation of the NCEP panel identifies LDL-C as the primary target for cholesterol-lowering therapy, it also suggests that the sum of LDL-C and VLDL-C (also referred to as “non-HDL-cholesterol”) should be a secondary target for cholesterol-lowering therapies, particularly in patients with high serum triglycerides (≦200 mg/dL; ≧2.3 mmol/L). Non-HDL-C goals recommended were <130 mg/dL for patients with existing CHD or CHD risk equivalent, <160 mg/dL for patients with two or more risk factors for CHD, and <190 mg/dL for patients with 0-1 CHD risk factors. Thus, a VLDL-C level of 30 mg/dL is considered normal; a circulating level of VLDL-C above 30 mg/dL is a high circulating level of VLDL-C.

Measurement of circulating levels of total cholesterol, LDL-C, HDL-C, and VLDL-C can be measured by standard, routine laboratory methods. These methods are well known and some of these methods are described elsewhere herein. Total blood cholesterol is generally taken to be the sum of LDL-C, HDL-C and VLDL-C. Typically, LDL-C can be directly measured by centrifugation techniques or can be estimated indirectly (in individuals with serum triglyceride levels <4.5 mmol/L) by subtracting the HDL-C and VLDL-C from the total plasma cholesterol. HDL-C is typically determined after chemical precipitation of VLDL-C and LDL-C from the sample. VLDL-C can be estimated in some cases by dividing the plasma triglyceride level by 5.

Ratios of various cholesterol components are often used as convenient indicators of the need for therapeutic intervention. For example, the total cholesterol/HDL-C ratio is ideally 3.5/1 or below; the HDL-C/LDL-C ratio is ideally 0.4/1 or more; the LDL-C/HDL-C ratio is ideally 2.5/1 or less.

The circulating level of total cholesterol, LDL-C, HDL-C, VLDL-C and other lipid components means the level of those components found in the blood, and can be determined from a blood, plasma, or serum sample as is standard practice. Reference herein to total cholesterol, LDL-C, HDL-C, VLDL-C and/or triglycerides refers to the circulating level of these components. Reference to LDL, HDL, and/or VLDL herein means LDL cholesterol (or LDL-C) HDL cholesterol (or HDL-C), and/or VLDL cholesterol (or VLDL-C), respectively.

Reference to “cholesterol” not prefaced by LDL, HDL, or VLDL, means total cholesterol. “Total cholesterol” refers to the total amount of cholesterol (typically measured in mg/dL) present in the blood in any and all types of lipoprotein particle.

### Method and Uses

The present invention provides methods for regulating cholesterol metabolism, including regulating cholesterol biosynthesis, uptake, processing, storage, transport, clearance, and utilization, in a subject by administering to the subject an effective amount of a compound that inhibits HIF hydroxylase activity. The present invention additionally provides methods for achieving and/or maintaining cholesterol homeostasis in a subject by administering to the subject an effective amount of a compound that inhibits HIF hydroxylase activity, thereby achieving and/or maintaining cholesterol homeostasis in the subject. Regulation of cholesterol metabolism and achieving/maintaining cholesterol homeostasis in the methods of the present invention can be effected by modulating the circulating levels of LDL-C, and/or of VLDL-C, alone or in combination with modulating the circulating levels of HDL-C, serum triglycerides, or other lipid components.

In one embodiment the present invention provides a method fbr treatment of high cholesterol in a subject in need thereof, by reducing the circulating level of total cholesterol and particularly by reducing the circulating level of LDL-C and/or VLDL-C, and/or increasing the ratio of HDL-C/LDL-C, the method comprising administering to the subject an effective amount of a compound that inhibits HIF hydroxylase activity.

In a certain embodiment, the present invention provides a method of reducing the circulating level of low density lipoprotein cholesterol (LDL-C) in a subject in need thereof, the method comprising administering to the subject an effective amount of a compound that inhibits HIF hydroxylase activity, whereby the circulating level of LDL-C in said subject is reduced. The subject in need of treatment in the method typically is a subject having a high circulating level of total cholesterol and/or a high circulating level of LDL-cholesterol, but other subjects may be suitable as described elsewhere herein. The circulating level of total cholesterol or LDL-C in the blood of a subject can be measured by routine laboratory protocols. Typically, the circulating level of LDL-C in the subject is reduced (from the pretreatment level) by at least 10%, by at least 20%, by at least 25%, by at least 30%, by at least 40%, or more by the practice of the present invention. The circulating level of LDL-C in the subject may be reduced (from the pretreatment level) by at least 10 mg/dL, by at least 20 mg/dL, by at least 25 mg/dL, by at least 30 mg/dL, or more. The compound is administered in an effective amount, typically a dose of from 0.01 mg/kg to 50 mg/kg, at specified time intervals, preferably 1, 2, or 3 times a week or 1, 2, or 3 times a month, but may be administered as frequently as once a day or once every other day. Any convenient mode of administration is suitable, oral administration is preferred. The circulating level of low density lipoprotein cholesterol will typically be reduced within 24 hours after administration of the compound but may require longer time period, eg, 2 days up to one week. Reduction of the LDL-C will continue with continued dosing. A desirable level of circulating LDL-C can be maintained by periodic monitoring of the LDL-C and dose adjustment of the compound. Typically the LDL-C level will return to the pre-treatment level when treatment is discontinued. Optionally, the method comprises the further step of monitoring the LDL-C level in the subject to confirm that the level is reduced, and further may include the additional step of adjusting the administration of the compound to maintain the LDL-C level within a desirable range.

In one embodiment the invention provides method of reducing the circulating level of very low density lipoprotein cholesterol (VLDL-C) in a subject in need thereof, the method comprising administering to the subject an effective amount of a compound that inhibits HIF hydroxylase activity, whereby the circulating level of VLDL-C in said subject is reduced. The subject in need of treatment in the method typically is a subject having a high circulating level of total cholesterol and/or a high circulating level of LDL-cholesterol and/or a high circulating level of VLDL-cholesterol, but other subjects may be suitable as described elsewhere herein. The circulating level of total cholesterol or LDL-C or VLDL-C in the blood of a subject can be measured by routine laboratory protocols. Typically, the circulating level of VLDL-C in the subject is reduced (from the pretreatment level) by at least 10%, by at least 20%, by at least 25%, by at least 30%, by at least 40%, or more by the practice of the present invention. The circulating level of VLDL-C in the subject may be reduced (from the pretreatment level) by at least 5 mg/dL, by at least 10 mg/dL, by at least 15 mg/dL, by at least 20 mg/dL, or more. The compound is administered in an effective amount, typically a dose of from 0.01 mg/kg to 50 mg/kg, at specified time intervals, preferably 1, 2, or 3 times a week or 1, 2, or 3 times a month, but may be administered as frequently as once a day or once every other day. Any convenient mode of administration is suitable, oral administration is preferred. The circulating level of very low density lipoprotein cholesterol will typically be reduced within 24 hours after administration of the compound but may require longer time period, eg, 2 days up to one week. Reduction of the VLDL-C will continue with continued dosing. A desirable level of circulating VLDL-C can be maintained by periodic monitoring of the VLDL-C and dose adjustment of the compound.

Typically the VLDL-C level will return to the pre-treatment level when treatment is discontinued. Optionally, the method comprises the further step of monitoring the VLDL-C level in the subject to confirm that the level is reduced, and further may include the additional step of adjusting the administration of the compound to maintain the VLDL-C level within a desirable range.

In another embodiment the invention provides method of reducing the circulating levels of very low density lipoprotein cholesterol (VLDL-C) and low density lipoprotein cholesterol (LDL-C) in a subject in need thereof, the method comprising administering to the subject an effective amount of a compound that inhibits HIF hydroxylase activity, whereby the circulating levels of both LDL-C and VLDL-C in said subject are reduced. This method is carried out identically to the separate methods of reducing the LDL-C and reducing the VLDL-C as described herein.

In another embodiment the invention provides a method of reducing the circulating level of total cholesterol (or the circulating levels of LDL-C and/or VLDL-C) in a subject under treatment for high cholesterol with a separate cholesterol-lowering agent, the method comprising administering to the subject an effective amount of a compound that inhibits HIF hydroxylase activity, whereby the circulating level of total cholesterol is reduced. It was found that application of the method of the invention was beneficial in reducing total cholesterol, LDL-C and/or VLDL-C in a subject that was already on a therapeutic regimen for treating high cholesterol, for example with statins. For some subjects, treatment with conventional cholesterol-lowering medication is not effective or sufficient to achieve desirably low levels of LDL-C, VLDL-C, or total cholesterol. For these subjects, administration of a compound that inhibits HIF hydroxylase activity can result in an additional reduction in LDL-C, VLDL-C, and/or total cholesterol levels. In some embodiments, the subject is under treatment with a separate cholesterol-lowering agent selected from the group consisting of a HMGCoA reductase inhibitor (for example, statins such as, simvastatin, atorvastatin, rosuvastatin, pravastatin, lovastatin, fluvastatin), a nicotinic acid (for example, niacins), a fibric acid (for example, fenofibrate, gemfibrozil), and a bile acid-binding resin (for example, cholestyramine, colesevelam, colestipol). Optionally, the method comprises the further step of monitoring the total cholesterol, LDL-C, and/or VLDL-C level in the subject to confirm that the level is reduced, and further may include the additional step of adjusting the administration of the compound to maintain the total cholesterol, LDL-C, and/or VLDL-C level within a desirable range.

In a further embodiment, the present invention provides a method of increasing the ratio of HDL-C/LDL-C in a subject in need thereof, the method comprising administering to the subject an effective amount of a compound that inhibits HIF hydroxylase activity, whereby the ratio of HDL-C/LDL-C in said subject is increased. The increase in the ratio of HDL-C/LDL-C can be accomplished by the present methods by reducing the circulating levels of LDL-C or by raising the circulating levels of HDL-C, or both. As will be apparent, the effect of increasing the HDL-C/LDL-C is identical to decreasing the LDL-C/HDL-C ratio, therefore the invention also provides a method of decreasing the ratio of LDL-C/HDL-C in a subject in need thereof, the method comprising administering to the subject an effective amount of a compound that inhibits HIF hydroxylase activity, whereby the ratio of LDL-C/HDL-C in said subject is decreased.

In another embodiment, the present invention provides a method of decreasing the ratio of total cholesterol/HDL-C in a subject having high circulating level of total cholesterol, the method comprising administering to the subject an effective amount of a compound that inhibits HIF hydroxylase activity, whereby the ratio of total cholesterol/HDL-C in said subject is decreased. The decrease in the ratio of total cholesterol/HDL-C can be accomplished in the present methods by reducing the circulating levels of LDL-C or by raising the circulating level of HDL-C, or both.

In particular embodiments, the present invention contemplates methods for altering expression of a cholesterol regulatory factor in a subject by administering to the subject an effective amount of a compound that inhibits HIF hydroxylase activity, thereby altering expression of the cholesterol regulatory factor in the subject. In particular embodiments, the present invention contemplates methods for altering expression of a cholesterol biosynthetic enzyme in a subject by administering to the subject an effective amount of a compound that inhibits HIF hydroxylase activity, thereby altering expression of the cholesterol biosynthetic enzyme in the subject.

### Subject

In various embodiments, the subject is a cell, tissue, or organ. In other embodiments, the subject is an animal, preferably a mammal, most preferably a human. When the subject is a cell, the invention specifically contemplates that the cell can be an isolated cell, either prokaryotic or eukaryotic. In the case that the subject is a tissue, the invention specifically contemplates both endogenous tissues and in vitro tissues, e.g., tissues grown in culture. In preferred embodiments, the subject is an animal, particularly, an animal of mammalian species including rat, rabbit, bovine, ovine, porcine, murine, equine, and primate species. In a most preferred embodiment, the subject is human.

In general, a suitable subject for practice of the method of the present invention includes any subject having a circulating level of cholesterol (including total cholesterol, LDL-C, HDL-C, and/or VLDL-C). Suitable subjects include any mammalian subjects, in particular, human subjects.

In certain embodiments of the present methods, the suitable subject will be one having a high circulating level of total cholesterol. As described elsewhere herein, a high circulating level of total cholesterol can be a total blood cholesterol of 240 mg/dL and higher, or can be a total blood cholesterol of 200 mg/dL and higher, or can be any level of total blood cholesterol that is determined to be higher than the recommended or desirable level for the particular subject based on currently prevailing best medical practices and guidelines for total blood cholesterol.

In certain embodiments of the present methods, the suitable subject will be one having a high circulating level of LDL-cholesterol. As described elsewhere herein, a high circulating level of LDL-C can be a blood LDL-C of 70 mg/dL and higher, or can be a blood LDL-C of 100 mg/dL and higher, or can be a blood LDL-C of 130 mg/dL and higher, or can be a blood LDL-cholesterol of 160 mg/dL and higher, or can be any level of blood LDL cholesterol that is determined to be higher than the recommended or desirable level for the particular subject based on currently prevailing best medical practices and guidelines for blood LDL cholesterol levels.

“High cholesterol” in a subject means a high circulating level of total cholesterol, a high circulating level of LDL-C, or both. The subject may have been previously diagnosed as having for example, hyperlipidemia, hypercholesterolemia, etc.

In certain embodiments of the present methods, the suitable subject will be one having a low circulating level of HDL cholesterol. As described elsewhere herein, a low circulating level of HDL-C can be a blood HDL-C of 50 mg/dL and lower, or can be a blood HDL-C of 40 mg/dL and lower, or can be any level of blood HDL cholesterol that is determined to be lower than the recommended level for the particular subject based on currently prevailing best medical practices and guidelines for blood HDL cholesterol.

In certain embodiments of the present methods, a suitable subject will have one or more of a high circulating level of total cholesterol, a high circulating level of LDL-cholesterol, and a low circulating level of HDL-cholesterol. In certain embodiments of the present methods, a suitable subject will have a high serum triglyceride level, for example, a serum triglyceride level of 150 mg/dL or more, or of 200 mg/dL or more.

Certain disorders and genetic or environmental conditions may affect the circulating levels of cholesterol, and suitable subjects for the present methods may be ones having such disorders or conditions, for example, a subject having coronary heart disease, diabetes, atherosclerosis or clinical forms of atherosclerotic disease (e.g., PAD, abdominal aortic aneurysm, and symptomatic carotid artery disease), hypertension, a circulating level of HDL-C below 40 mg/dL, family history of premature CHD, or a history of cigarette smoking.

Suitable subjects also include a subject with chronic kidney disease, a subject with end stage renal disease, a subject with anemia, or a subject on dialysis.

### Compounds

A compound for use in the methods, uses, or medicaments provided herein is one that inhibits the activity of a hypoxia-inducible factor (HIF) hydroxylase enzyme. The compound that inhibits HIF hydroxylase activity can be composed of polynucleotides (e.g., antisense inhibitors of one or more HIF hydroxylase); polypeptides; antibodies (e.g. antibodies to one or more HIF hydroxylase); other proteins; carbohydrates; fats; lipids; and organic and inorganic substances, e.g., small molecules, etc. In a preferred embodiment, the compound that inhibits HIF hydroxylase activity is small molecule compound.

A compound that inhibits the activity of a HIF hydroxylase enzyme refers to any compound that reduces, eliminates, or attentuates the activity of at least one HIF hydroxylase enzyme. Suitably the compound will inhibit the activity of more than one HIF hydroxylase enzyme, for example, will inhibit PHD1, PHD2, and PHD3. Methods for determining whether a compound inhibits HIF hydroxylase activity are well known in the art and a number of techniques are described herein

Functionally, HIF hydroxylase inhibitors for use in the methods of the present invention are defined by their ability to inhibit an activity of a 2-oxoglutarate dioxygenase enzyme, wherein the enzyme has specific activity toward hypoxia inducible factor. Such compounds are often referred to as HIF hydroxylase inhibitors or prolyl hydroxylase inhibitors or “PHI”s. Preferably, the PHIs for use in the invention are small molecule compounds. A compound that inhibits the activity of a HIF hydroxylase enzyme may additionally show inhibitory activity toward one or more other 2-oxoglutarate- and iron-dependent dioxygenase enzymes, e.g. factor inhibiting HIF (FIH; GenBank Accession No. AAL27308), procollagen prolyl 4-hydroxylase (CP4H), etc.

In some embodiments the compound that inhibits HIF hydroxylase activity is a heterocyclic carboxamide. In some embodiments the compound that inhibits HIF hydroxylase activity is a heterocyclic carbonyl glycine. In some embodiments the compound that inhibits HIF hydroxylase activity is a structural mimetic of 2-oxoglutarate. In some embodiments the compound that inhibits HIF hydroxylase activity is an isoquinoline carboxamide. In some embodiments the compound that inhibits HIF hydroxylase activity is a compound of Formula I. In some embodiments the compound that inhibits HIF hydroxylase activity is a compound of Formula II. In some embodiments the compound that inhibits HIF hydroxylase activity is a compound of Formula III. In some embodiments the compound that inhibits HIF hydroxylase activity is a compound of Formula IV.

In particular embodiments, compounds used in the present methods and medicaments provided herein are structural mimetics of 2-oxoglutarate (2-OG), wherein the compound inhibits the target HIF prolyl hydroxylase enzyme competitively with respect to 2-oxoglutarate and noncompetitively with respect to iron. PHIs are typically heterocyclic carboxamide compounds, especially heterocyclic carbonyl glycine derivatives, and may be, for example, pyridine, pyrimidine, pyridazine, naphthyridine, pyrrolopyridine, pyrrolo-pyridazine, thiazolopyridine, isothiazolopyridine, quinoline, isoquinoline, cinnoline, beta-carboline, quinolone, thienopyridine, chromene, or thiochromene carboxamides.

Compounds that inhibit HIF prolyl hydroxylase are known in the art and are described in, inter alia, U.S. Pat. Nos. 5,658,933; 5,620,995; 5,719,164; 5,726,305; 6,093,730; 7,323,475; U.S. application Ser. No. 12/544,861; U.S. Patent Application Publication Nos. 2006/0199836; 2007/0298104; 2008/0004309; and PCT publication Nos. WO2009/073669; WO2009/089547; WO2009/100250; U.S. Patent Application Publication 2003/0176317, U.S. Patent Application Publication 2003/0153503, U.S. Pat. No. 7,323,475, U.S. Patent Application Publication 2006/0199836, U.S. Pat. No. 7,928,120, U.S. Pat. No. 7,696,223, U.S. Patent Application Publication 2010/0303928, U.S. Patent Application Publication 2010/0330199, U.S. Patent Application Publication 2010/0331400, U.S. Patent Application Publication 2010/0047367, PCT Application No. PCT/US2009/064065, U.S. Pat. No. 7,897,612, U.S. Pat. No. 7,608,621, U.S. Pat. No. 7,728,130, U.S. Pat. No. 7,635,715, U.S. Pat. No. 7,569,726, U.S. Pat. No. 7,811,595; U.S. Pat. No. 8,217,043; U.S. Pat. No. 8,269,008; U.S. Pat. No. 8,324,405; U.S. Patent Application Publication 2011/0305776; US 2007/0299086; US 2011/0111058; US 2011/0110961; WO 07/070,359; US 2009/0111806; US 2009/0093483; US 2009/0156605; US 2009/0088475; US 2009/0099171; WO 08/137,060; US 2009/0156633; US 2010/0035906; WO 08/049,538; WO 08/067,871; US 2010/0093803; US 2009/269420; WO 11/006,355; WO 11/106,226; US 2011/028507; WO 10/018,458; WO 11/056,725; WO 11/049,126; WO 11/049,127; WO 07/038,571; US 2009/0082357; US 2009/0176825; US 2010/0113444; U.S. Ser. No. 08/017,1756; WO 08/089,052; WO 09/039,321; WO 09/039,322; US 2009/0176825; WO 09/049,112; US 2010/0305154; US 2010/0305133; US 2010/0298324; WO 09/134,847; US 2011/0039895; US 2011/0098324; US 2011/0160227; WO 10/022,308; US 2011/0144167; WO 10/059,549; WO 10/059,552; WO 10/059,555; US 2011/0046132; WO 09/134,754; US 2010/0204226; WO 2012/021830; US 2011/0077267; US 2012/004197; US 2010/0056563; US 2010/0137297; US 2010/0331358; US 2011/009425; US 2011/009406; U.S. Ser. No. 09/023,9876; US 2011/0152304; WO 10/147,776; WO 11/002,623; WO 11/002,624; WO 11/133,444; WO 11/130,908; WO 10/076,524; WO 10/076,525; WO 11/045,811; US 2011/0130414; WO 11/048,611; and US 2009/0048294. The foregoing patents and patent applications are incorporated in their entireties herein. Other prolyl hydroxylase inhibitors are well known and have been described in the art.

Methods of determining if any particular compound inhibits HIF prolyl hydroxylase are well known, for example, the methods described in U.S. Pat. No. 7,323,475. The inhibitory activity of a compound can be conveniently evaluated and compared by determining the IC₅₀ for one or more of the HIF prolyl hydroxylase enzymes. The IC₅₀ for any compound for each of the HIF prolyl hydroxylase enzymes can be determined in the assays described herein. For example, for Compound A, the IC₅₀s for PHDI, PHD2, and PHD3 are very similar and are all in the micromolar range from about 0.2 to 2 μM. Typically, the compound that inhibits HIF hydroxylase will exhibit similar IC₅₀ for each of the PHD enzymes.

Compounds that inhibit HIF hydroxylase are known to increase endogenous erythropoietin typically resulting in an increase in hemoglobin (International Patent Application Publication WO03/053997). For the uses of the present invention, the compounds that inhibit HIF hydroxylase are preferably used at doses that provide a minimal increase in endogenous erythropoietin and/or hemoglobin.

In certain embodiments, the HIF hydroxylase inhibitor compounds used in the methods of the invention are selected from a compound of the formula (I)

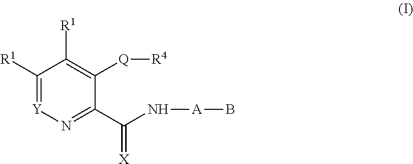

wherein
A is 1,2-arylidene, 1,3-arylidene, 1,4-arylidene; or (C₁-C₄)-alkylene, optionally substituted by one or two halogen, cyano, nitro, trifluoromethyl, (C₁-C₆)-alkyl, (C₁-C₆)-hydroxyalkyl, (C₁-C₆)-alkoxy, —O—[CH₂]ₓ—C₁H_{(2f+l-g)}Hal_{g}, (C₁-C₆)-fluoroalkoxy, (C₁-C₈)-fluoroalkenyloxy, (C₁-C₈)-fluoroalkynyloxy, —OCF₂Cl, —O—CF₂—CHFCl; (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N—(C₁-C₄)-alkylcarbamoyl, N,N-di-(C₁-C₄)-alkylcarbamoyl, (C₁-C₆)-alkylcarbonyloxy, (C₃-C₈)-cycloalkyl, phenyl, benzyl, phenoxy, benzyloxy, anilino, N-methylanilino, phenylmercapto, phenylsulfonyl, phenylsulfinyl, sulfamoyl, N—(C₁-C₄)-alkylsulfamoyl, N,N-di-(C₁-C₄)-alkylsulfamoyl; or by a substituted (C₆-C₁₂)-aryloxy, (C₇-C₁₁)-aralkyloxy, (C₆-C₁₂)-aryl, (C₇-C₁₁)-aralkyl radical, which carries in the aryl moiety one to five identical or different substituents selected from halogen, cyano, nitro, trifluoromethyl, (C₁-C₆)-alkyl, (C₁-C₆)-alkoxy, —O—[CH₂]ₓ—C_{f}H_{(2f+l-g)}Hal_{g}, —OCF₂Cl, —O—CF₂—CHFCl, (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N—(C₁-C₄)-alkylcarbamoyl, N,N-di-(C₁-C₄)-alkylcarbamoyl, (C₁-C₆)-alkylcarbonyloxy, (C₃-C₈)-cycloalkyl, sulfamoyl, N—(C₁-C₄)-alkylsulfamoyl, N,N-di-(C₁-C₄)-alkylsulfamoyl; or wherein A is —CR⁵R⁶ and R⁵ and R⁶ are each independently selected from hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer;
B is —CO₂H, —NH₂, —NHSO₂CF₃, tetrazolyl, imidazolyl, 3-hydroxyisoxazolyl, —CONHCOR′″, —CONHSOR′″, CONHSO₂R′″, where R′″ is aryl, heteroaryl, (C₃-C₇)-cycloalkyl, or (C₁-C₄)-alkyl, optionally monosubstituted by (C₆-C₁₂)-aryl, heteroaryl, OH, SH, (C₁-C₄)-alkyl, (C₁-C₄)-alkoxy, (C₁-C₄)-thioalkyl, (C₁-C₄)-sulfinyl, (C₁-C₄)-sulfonyl, CF₃, Cl, Br, F, I, NO2, —COOH, (C₂-C₅)-alkoxycarbonyl, NH₂, mono-(C₁-C₄-alkyl)-amino, di-(C₁-C₄-alkyl)-amino, or (C₁-C₄)-perfluoroalkyl; or wherein B is a CO₂-G carboxyl radical, where G is a radical of an alcohol G-OH in which G is selected from (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical, where the alkenyl, cycloalkenyl, alkynyl, and alkenynyl radicals contain one or more multiple bonds; (C₆-C₁₆)-carbocyclic aryl radical, (C₇-C₁₆)-carbocyclic aralkyl radical, heteroaryl radical, or heteroaralkyl radical, wherein a heteroaryl radical or heteroaryl moiety of a heteroaralkyl radical contains 5 or 6 ring atoms; and wherein radicals defined for G are substituted by one or more hydroxyl, halogen, cyano, trifluoromethyl, nitro, carboxyl, (C₁-C₁₂)-alkyl, (C₃-C₈)-cycloalkyl, (C₅-C₈)-cycloalkenyl, (C₆-C₁₂)-aryl, (C₇-C₁₆)-aralkyl, (C₂-C₁₂)alkenyl, (C₂-C₁₂)-alkynyl, (C₁-C₁₂)-alkoxy, (C₁-C₁₂)-alkoxy-(C₁-C₁₂)-alkyl, (C₁-C₁₂)-alkoxy-(C₁-C₁₂)-alkoxy, (C₆-C₁₂)-aryloxy, (C₇-C₆)-aralkyloxy, (C₁-C₈)-hydroxyalkyl, —O—[CH₂]ₓ—C_{f}H_{(2f+1-g)}, —OCF₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₁₂)-alkenylcarbonyl, (C₂-C₁₂)-alkynylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, acloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆) aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di(C₁-C₁₂)-alkylcarbamnoyl, N—(C₃-C₈)-cycloalkyl-carbamoyl, N—(C₆-C₁₆)-arylcarbamioyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₀)-alkoxy-(C₁-C₁₀)-alkyl)-carbamoyl, N—((C₆-C₁₂)-aryloxy-(C₁-C₁₀)alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₆)-aryloxy-(C₁-C₁₀)-alkyl)-carbamoyl, N—(C₁-C₁₀)-alkyl-N—((C₇-C₆)-aralkyloxy-(C₁-C₁₀)-alkyl)-carbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-aylcarbamoyloxy, N(C₁-C₁₀)-alkyl-N—(C₇-C₁₆)-aralkylcarbaxnoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆)-arakyloxy-(C₁-C₁₀)-alkyl)-carbamoyloxy, amino, (C₁-C₁₂)-alkylamino, di-(C₁-C₁₂)-alkylamino, (C₃-C₈)-cycloalkylamino, (C₂-C₁₂)-alkenylamino, (C₂-C₁₂)-alkynylamino, N—(C₆-C₁₂)-arylamino, N—(C—C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₂-C₁₂)-alkylcarbonylamino, (C₃-C₈)-cycloalkylcarbonylamino, (C₆-C₁₂) arylcarbonylamino, (C₇-C₁₆)-aralkylcarbonylamino, (C₁-C₁₂)-alkylcarbonyl-N—(C₁-C₁₀)-alkylamino, (C₃-C₈)-cycloalkylcarbonyl-N—(C₁-C₁₀)-alkylamino, (C₆-C₁₂)-arylcarbonyl-N—(C₁-C₁₀)alkylamino, (C₇-C₁₁)-aralkylcarbonyl-N—(C₁-C₁₀)-alkylamino, (C₁-C₁₂)-alkylcarbonylamino (C₁-C₈)-alkyl, (C₃-C₈)-cycloalkylcarbonylamino-(C₁-C₈)alkyl, (C₆-C₁₂)-arylcarbonylamino-(C₁-C₈)-alkyl, (C₇-C₁₂)-aralkylcarbonylamino(C₁-C₈)-alkyl, amino-(C₁-C₁₀)-alkyl, N—(C₁-C₁₀) alkylamino-(C₁-C₁₀)-alkyl, N,N-di-(C₁-C₁₀)-alkylamino-(C₁-C₁₀)-alkyl, (C₃-C₈)cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₂)-arylsulfonyl, (C₆-C₁₆)-aralkylmercapto, (C₆-C₁₆)-aralkylsulfinyl, (C₇-C₁₆)-aralkylsulfbnyl, sulfamoyl, N—(C₁-C₁₀)-alkylsulfamoyl, N,N-di(C₁-C₁₀)-alkylsulfamoyl, (C₃-C₈)-cycloalkylsulfamoyl, N—(C₆-C₁₂)-alkylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₆)-aralkylsulfamoyl, (C₁-C₁₀)-atkylsulfonamido, N—((C₁-C₁₀)-alkyl)-(C₁-C₁₀)-alkylsulfonamido, (C₇-C₁₆)-aralkylsulfonamido, or N—((C₁-C₁₀)-alkyl-(C₇-C₁₆)-aralkylsulfonamido; wherein radicals which are aryl or contain an aryl moiety, may be substituted on the aryl by one to five identical or different hydroxyl, halogen, cyano, trifluoromethyl, nitro, carboxyl, (C₁-C₁₂)-alkyl, (C₃-C₈)-cycloalkyl, (C₆-C₁₂)-aryl, (C₇-C₁₆)-aralkyl, (C₁-C₁₂)-alkoxy, (C₁-C₁₂)-alkoxy-(C₁-C₁₂)alkyl, (C₁-C₁₂)-alkoxy-(C₁-C₁₂)alkoxy, (C₆-C₁₂)-aryloxy, (C₇-C₁₆)-aralkyloxy, (C₁-C₈)-hydroxyalkyl, (C₁-C₁₂)-alkylcarbonyl, (C₃-C₈)-cycloalkyl-carboxyl, (C₆-C₁₂)-arylcarbonyl, (C₇-C₁₆) aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkcynyloxycarbonyl, (C₁-C₁₂)-alkylcazbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₁-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbaznoyl, N,N-di-(C₁-C₁₂)-alkylcabamoyl, N—(C₃-C₈)-cycloalkylcarbamoyl, N—(C₆-C₁₂)-arylcarbamoyl, N—(C₇-C₁₆)-arakylcarbamnoyl, N—(C₁-C₁₀)-alkyl-N—(C₆-C₁₂)-arylcarbamoyl, N—(C₁-C₁₀)-alkyl-N—(C₇-C₁₆)-aralkylcarbamoyl, N—((C₁-C₁₀)-alkoxy-(C₁-C₁₀)-alkyl)-carbamoyl, N—((C₆-C₁₂)-aryloxy-(C₁-C₁₀)-alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₂)-aryloxy-(C₁-C₁₀)-alkyl)-carbamoyl, N—(C₁-C₁₀)-alkyl-N—((C₇-C₁₆)-aralkyloxy-(C₁-C₁₀)-alkyl)-carbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆-aralkyloxy-(C₁-C₁₀)-alkyl)-carbamoyloxy, amino, (C₁-C₁₂)-alkylamino, di-(C₁-C₁₂)-alkylamino, (C₃-C₈)-cycloalkylamino, (C₃-C₁₂)-alkynylamino, (C₃-C₁₂)-alkynylamino, N—(C₆-C₁₂)-arylamino, N—(C₇-C₁₁)-aralkylamino, N-alkylaralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkylcarbonylamino, (C₃-C₈)-cycloalkylcarbonylamino, (C₆-C₁₂)-aylcarbonylamino, (C₇-C₁₆)-alkylcarbonylamino, (C₁-C₁₂)-alkylcarbonyl-N—(C₁-C₁₀)-alkylamino, (C₃-C₈)-cycloalkylcarbonyl-N—(C₁-C₁₀)-alkylamino, (C₆-C₁₂)-arylcarbonyl-N—(C₁-C₁₀)-alkylamino, (C₇-C₁₁)-aralkylcarbonyl-N—(C₁-C₁₀)-alkylamino, (C₁-C₁₂)-alkylcarbonylamino-(C₁-C₈)-alkyl, (C₃-C₈)-cycloalkylcarbonylamino-(C₁-C₈)-alkyl, (C₆-C₁₂)-arylcarbonylamino-(C₁-C₈)-alkyl, (C₇-C₁₆)-aralkylcarbonylamino-(C₁-C₈)-alkyl, amino-(C₁-C₁₀)-alkyl, N—(C₁-C₁₀)-alkylamino-(C₁-C₁₀)-alkyl, N,N-di-(C₁-C₁₀-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₂)-arylmercapto, (C₆-C₁₂)-arylsulfinyl, (C₆-C₁₂)-arylsulfbnyl, (C₇-C₁₆)-aralkylmercapto, (C₇-C₁₆)-aralkylsulfinyl, or (C₇-C₁₆)-aralkylsulfonyl;
X is O or S;
Q is O, S, NR′, or a bond;
where, if Q is a bond, R⁴ is halogen, nitrite, or trifluoromethyl;
or where, if Q is O, S, or NR′, R⁴ is hydrogen, (C₁-C₁₀)-alkyl radical, (C₂-C₁₀)-alkenyl radical, (C₂-C₁₀)-alkynyl radical, wherein alkenyl or alkynyl radical contains one or two C—C multiple bonds; unsubstituted fluoroalkyl radical of the formula —[CH₂]ₓ—C₁H_{(2f+1-g)}—F_{g}, (C₁-C₈)-alkoxy-(C₁-C₆)-alkyl radical, (C₁-C₆)-alkoxy-(C₁-C₄)-alkoxy-(C₁-C₄)-alkyl radical, aryl radical, heteroaryl radical, (C₁-C₁₁)-aralkyl radical, or a radical of the formula Z

—[CH₂]ᵥ—[O]_{w}—[CH]ₗ-E (Z)

where
E is a heteroaryl radical, a (C₃-C₈)-cycloalkyl radical, or a phenyl radical of the formula F

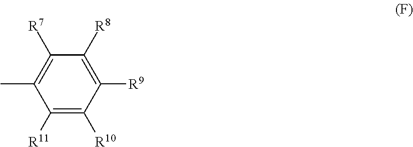

v is 0-6,
w is 0 or 1
t is 0-3, and
R⁷, R⁸, R⁹, R¹⁰, and R¹¹ are identical or different and are hydrogen, halogen, cyano, nitro, trifluoromethyl, (C₁-C₆)-alkyl, (C₃-C₈)-cycloalkyl, (C₁-C₆)-alkoxy, —O—[CH₂]ₓ—C_{f}H_{(2f+1-g)}—F_{g}, —OCF₂—Cl, —O—CF₂—CHFCl, (C₁-C₆)-alkylmercapto, (C₁-C₆)-hydroxyalkyl, (C₁-C₆)-alkoxy-(C₁-C₆)-alkoxy, (C₁-C₆)-alkoxy-(C₁-C₆)-alkyl, (C₁-C₆)-alkylsulfinyl, (C₁-C₆)-alkylsulfonyl, (C₁-C₆)-alkylcarbonyl, (C₁-C₈)-alkoxycarbonyl, carbamnoyl, N—(C₁-C₈)-alkylcarbamoyl, N,N-di-(C₁-C₈)-alkylcarbamoyl, or (C₇-C₁₁)-aralkylcarbamoyl, optionally substituted by fluorine, chlorine, bromine, trifluoromethyl, (C₁-C₆)-alkoxcy, N—(C₃-C₈)-cycloalkylcarbamoyl, N—(C₃-C₈)-cycloalkyl-(C₁-C₄)-alkylcarbamoyl, (C₁-C₆)-alkylcarbonyloxy, phenyl, benzyl, phenoxy, benzyloxy, NR^{Y}R^{Z} wherein R^{y} and R^{z} are independently selected from hydrogen, (C₁-C₁₂)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₃-C₁₀)-cycloalkyl, (C₃-C₁₂)-alkenyl, (C₃-C₁₂)-alkynyl, (C₆-C₁₂)-aryl, (C₇-C₁₁)-aralkyl, (C₁-C₁₂)-alkoxy, (C₇-C₁₂)aralkoxy, (C₁-C₁₂)-alkylcarbonyl, (C₃-C₈)-cycloalkylcarbonyl, (C₆-C₁₂) arylcarbonyl, (C₇-C₁₆)-aralkylcarbonyl; or further wherein R^{y} and R^{z} together are —[CH2]ₕ, in which a CH₂ group can be replaced by O, S, N—(C₁-C₄)-alkylcarbonylimino, or N—(C₁-C₄)-alkoxycarbonylimino; phenylmercapto, phenylsulfonyl, phenylsulfinyl, sulfamoyl, N—(C₁-C₈)-alkylsulfamoyl, or N,N-di-(C₁-C₈)-alkylsulfamoyl; or alternatively R⁷ and R⁸, R⁸ and R⁹, R⁹ and R¹⁰, or R¹⁰ and R¹¹, together are a chain selected from —[CH₂]ₙ— or —CH═CH—CH═CH—, where a CH₂ group of the chain is optionally replaced by O, S, SO, SO₂, or NR^{Y}; and n is 3, 4, or 5; and if E is a heteroaryl radical, said radical can carry 1-3 substituents selected from those defined for R⁷-R¹¹, or if E is a cycloalkyl radical, the radical can carry one substituent selected from those defined for R⁷-R¹¹;
or where, if Q is NR′, R⁴ is alternatively R″, where R′ and R″ are identical or different and are hydrogen, (C₆-C₁₂)-aryl, (C₇-C₁₁)-aralkyl, (C₁-C₈)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkylcarbonyl, optionally substituted (C₇-C₁₆)-aralkylcarbonyl, or optionally substituted C₆-C₁₂)-arylcarbonyl; or R′ and R″ together are —[CH₂]ₕ, in which a CH₂ group can be replaced by O, S, N-acylimino, or N—(C₁-C₁₀)-alkoxycarbonylimino, and h is 3 to 7;
Y is N or CR³;
R¹, R² and R³ are identical or different and are hydrogen, hydroxyl, halogen, cyano, trifluoromethyl, nitro, carboxyl, (C₁-C₂₀)-alkyl, (C₃-C₈)-cycloalkyl, (C₃-C₈)cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyloxy-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl-(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₇-C₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₁-C₂₀)-alkoxy-(C₁-C₁₂)-alkyl, (C₁-C₁₂)-alkoxy-(C₁-C₁₂)-alkoxy, (C₁-C₁₂)-alkoxy-(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₁₆)-hydroxyalkyl, (C₆-C₁₆)-aryloxy-(C₁-C₈)-alkyl, (C₇-C₁₆)-aralkoxy-(Cl—C₈)-alkyl, (C₆-C₂)-aryloxy-(C₁-C₈)-alkoxy-(C₁-C₆)-alkyl, (C₇-C₁₂)-aralkyloxy-(Cl—C₈)-alkoxy-(C₁-C₆)-alkyl, (C₂-C₂₀)-alkenyloxy-(C₁-C₆)-alkyl, (C₂-C₂₀)-alkynyloxy-(C₁-C₆)-alkyl, retinyloxy-(C₁-C₆)-alkyl, —O—[CH₂]ₓCfH_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl-(C₁-C₆)-alkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N—(C₁-C₆)-alkyl-N—((C₃-C₈)-cycloalkyl-(C₁-C₆)-alkyl)-carbamoyl, N-(+)-dehydroabictylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamnoyl, N—(C₁-C₁₀)-alkyl-N—(C₇-C₁₆)-aralkylcarbamoyl, N—((C₁-C₁₈)-alkoxy-(C₁-C₁₀)-alkyl) carbamoyl, N—((C₆-C₁₆)-aryloxy-(C₁-C₁₀)-alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₂)-aryloxy-(C₁-C₁₀)-alkyl)-carbamoyl, N—(C₁-C₁₀)-alkyl-N—((C₇-C₁₆)-aralkyloxy-(C₁-C₁₀-alkyl)-carbamoyl; CON(CH₂)ₕ, in which a CH₂ group can be replaced by O, S, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; a carbamoyl radical of the formula R

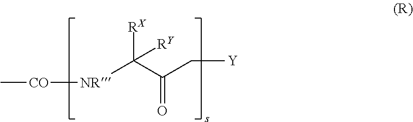

in which
R^{x} and R^{y} are each independently selected from hydrogen, (C₁-C₆)-alkyl, (C₃-C₇)-cycloalkyl, aryl, or the substituent of an α-carbon of an α-amino acid, to which the L- and D-amino acids belong,
s is 1-5.
T is OH, or NR*R**, and R*, R** and R*** are identical or different and are selected from hydrogen, (C₆-C₁₂)-aryl, (C₇-C₁₁)-aralkyl, (C₁-C₈)-alkyl, (C₃-C₈)-cycloalkyl, (+)-dehydroabietyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, optionally substituted (C₆-C₁₂)-aroyl; or R* and R** together are —[CH₂]ₕ, in which a CH₂ group can be replaced by O, S, SO, SO₂, N-acylamino, N—(C₁-C₁₀)-alkoxycarbonylimino, N—(C₁-C₈)-alkylimino, N—(C₃-C₈)-cycloalkylimino, N—(C₃-C₈)-cycloalkyl-(C₁-C₄)-alkylimino, N— (C₆-C₁₂)-arylimino, N—(C₇-C₁₆)-aralkylimino, N—(C₁-C₄-alkoxy-(C₁-C₆)-alkylimino, and h is from 3 to 7;
carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-c₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, (C₁-C₁₂)-alkanoylamino-(C₁-C₈)-alkyl, (C₃-C₈)cycloalkanoylaminio-(C₁-C₈)-alkyl, (C₆-C₁₂)-aroylamino-(C₁-C₈)-alkyl, (C₇-C₁₆)-aralkanoylamlino (C₁-C₈)-alkyl, amino-(C₁-C₁₀)-alkyl, N—(C₁-C₁₀)-alkylamino-(C₁-C₁₀)-alkyl, N,N-di(C₁-C₁₀)-alkylamino-(C₁-C₁₀)-alkyl, (C₃-C₈)-cycloalkylamino(C₁-C₁₀)-alkyl, (C₁-C₂₀)-alkylmercapto, (C₁-C₂₀)-alkylsulfinyl, (C₁-C₂₀)-alkylsulfonlyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, (C₁-C₁₂)-alkylmercapto-(C₁-C₆)-alkyl, (C₁-C₁₂)-alkylsulfinyl-(C₁-C₆)-alkyl, (C₁-C₁₂)-alkylsulfonyl-(C₁-C₆)-alkyl, (C₆-C₁₂)-arylmercapto-(C₁-C₆-alkyl, (C₇-C₁₆)-aralkylmercapto-(C₁-C₆)-alkyl, (C₇-C₁₆)-aralkylsulfinyl-(C₁-C₆)-alkyl, (C₇-C₁₆)-aralkylsulfonyl-(C₁-C₆)-alkyl, sulfamoyl, N—(C₁-C₁₀)-alkylsulfamoyl, N,N-di-(C₁-C₁₀)-alkylsulfamoyl, (C₃-C₅)-cycloalkylsulfamoyl, N—(C₆-C₁₂)-arylsulfbnmoyl, N—(C₇-C₁₆)-aralkylsulfamoyl, N—(C₁-C₁₀)-alkyl-N—(C₆-C₁₂)-arylsulfamoyl, N—(C₁-C₁₀)alkyl-N—(C₇-C₁₀)-aralkylsulfamoyl, (C₁-C₀)-alkylsulfonamido, N—((C₁-C₁₀)-alkyl)-(C₁-C₁₀)-alkylsulfonamido, (C₇-C₁₆)-aralkylsulfonamido, and N—((C₁-C₁₀)-alkyl-(C₇-C₁₆)aralkylsulfonamido; where an aryl radical may be substituted by 1 to 5 substituents selected from hydroxyl, halogen, cyano, trifluoromethyl, nitro, carboxyl, (C₁-C₁₆)-alkyl, (C₃-C₈)-cycloalkyl, (C₃-C₅)-cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyloxy-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl(C₁-C₈)-alkoxy-(C₁-C₆)-alkyl, (C₃-C₈)-oycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₂-C₁₆)-alkenyl, (C₁-C₁₂)-alkynyl, (C₁-C₁₆)-alkoxy, (C₁-C₁₆)-akenyloxy, (C₁-C₁₂)-alkoxy-(C₁-C₁₂)-alkyl, (C₁-C₁₂)-alkoxy-(C₁-C₁₂)-alkoxy, (C₁-C₁₂)-alkoxy(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₈)-hydroxyalkyl, (C₆-C₁₆)-aryloxy-(C₁-C₈)-alkyl, (C₁-C₁₆-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, —O—[CH₂]ₓC_{f}H_{(2f+1-g)}F_{g}, —OCF₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl-(C₁-C₆)-alkoxycarbonyl, (C₃-C₈)-cyctoalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-arakylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di(C₁-C₁₂)-alkylcarbamoyl, N—(C₃-C₈)-cycloalkylcarbamoyl, N,N-dicyclo-(C₃-C₈)-alkylcarbamioyl, N—(C₁-C₁₀)-alkyl-N—(C₃-C₈)-cycloalkylcarbamoyl, N—((C₃-C₈)-cycloalkyl-(C₁-C₆)-alkyl)carbamoyl, N—(C₁-C₆)-alkyl-N—((C₃-C₈)-cycloalkyl-(C₁-C₆)-alkyl)carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamnoyl, N—((C₁-C₁₆)-alkoxy-(C₁-C₁₀)-alkyl)carbamoyl, N—((C₆-C₁₆)-aryloxy-(C₁-C₁₀)-alkyl)carbamoyl, N—((C₇-C₁₆)-aralkyloxy-(C₁-C₁₀)-alkyl)carbamoyl, N—(C₁-C₁₀)-alkyl-N—((C₁-C₁₀)-alkoxy-(C₁-C₁₀)-alkyl)carbamoyl, N—(C₁-C₁₀)-alkyl-N—((C₆-C₁₂)-aryloxy-(C₁-C₁₀)-alkyl)carbamoyl, N—(C₁-C₁₀)-alkyl-N—((C₇-C₁₆)-aralkyloxy-(C₁-C₁₀)-alkyl)-carbamoyl, CON(CH)ₕ, in which a CH₂ group can be replaced by, O, S, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₆)-arylcarbamoyloxy, N—(C₇-C₁₆)-aralkylcarbamoyloxy, N—(C₁-C₁₀)-alkyl-N—(C₆-C₁₂)-arylcarbamoyloxy,N—(C₁-C₁₀)-alkyl-N—(C₇-C₁₆)-aralkylcarbamoyloxy, N—((C₁-C₁₀)-alkyl)carbamoyloxy, N—((C₆-C₁₂)-aryloxy-(C₁-C₁₀)-alkyl)carbamoyloxy, N—((C₇-C₁₆)-aralkyloxy-(C₁-C₁₀)-alkyl)carbamoyloxy,N—(C₁-C₁₀)-alkyl-N—((C₁-C₁₀)-alkoxy-(C₁-C₁₀)-alkyl)carbamoyloxy, N—(C₁-C₁₀)-alkyl-N—((C₆-C₁₂)-aryloxy-(C₁-C₁₀)-alkyl)carbamoyloxy, N—(C₁-C₁₀)-alkyl-N—((C₇-C₁₆)-aralkyloxy-(C₁-C₁₀)-alkyl)carbamoyloxy, amino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₇-C₁₀)-alkylamino, (C₆-C₂)-aroyl-N—(C₁-C₁₀)-alkylamino, (C₇-C₁₁)-aralkanoyl-N—(C₁-C₁₀)-alkylamino, (C₁-C₁₂)-alkanoylamino-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N—(C₁-C₁₀)-alkylamino-(C₁-C₁₀)-alkyl, N,N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₆)-arylsulfonyl, (C₇-C₁₆)-aralkylmercapto, (C₇-C₁₆)-aralkylsulfinyl, or (C₇-C₁₆)-aralkylsulfonyl;
or wherein R¹ and R², or R² and R³ form a chain [CH₂]ₒ, which is saturated or unsaturated by a C═C double bond, in which 1 or 2 CH₂ groups are optionally replaced by O, S, SO, SO₂, or NR′, and R′ is hydrogen, (C₆-C₁₂)-aryl, (C₁-C₈)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, or optionally substituted (C₆-C₁₂)-aroyl; and o is 3, 4 or 5;
or wherein the radicals R¹ and R², or R² and R₃, together with the pyridine or pyridazine carrying them, form a 5,6,7,8-tetrahydroisoquinoline ring, a 5,6,7,8-tetrahydroquinoline ring, or a 5,6,7,8-tetrahydrocinnoline ring;
or wherein R¹ and R², or R² and R³ form a carbocyclic or heterocyclic 5- or 6-membered aromatic ring;
or where R¹ and R², or R² and R³, together with the pyridine or pyridazine carrying them, form an optionally substituted heterocyclic ring systems selected from thienopyridines, furanopyridines, pyridopyridines, pyrimidinopyridines, imidazopyridines, thiazolopyridines, oxazolopyridines, quinoline, isoquinoline, and cinnoline; where quinoline, isoquinoline or cinnoline preferably satisfy the formulae Ia, lb and Ic:

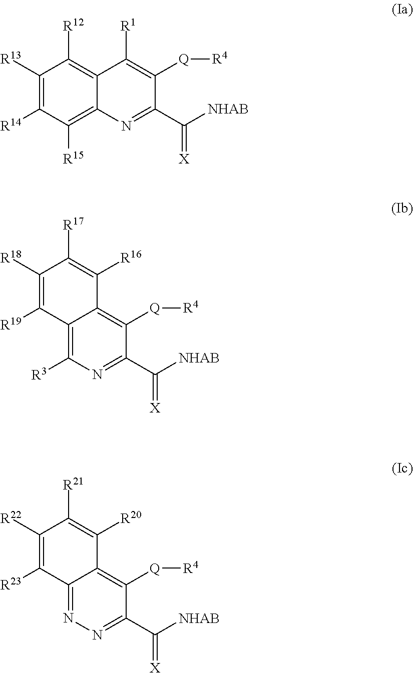

and the substituents R¹² to R²³ in each case independently of each other have the meaning of R¹, R² and R³;
or wherein the radicals R¹ and R², together with the pyridine carrying them, form a compound of Formula Id:

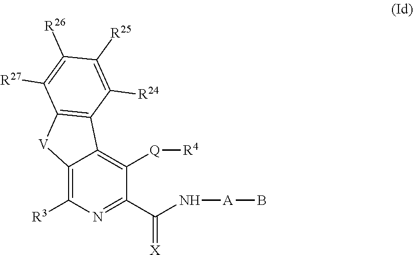

where V is S, O, or NR^{k}, and R^{k} is selected from hydrogen, (C₁-C₆)-alkyl, aryl, or benzyl; where an aryl radical may be optionally substituted by 1 to 5 substituents as defined above; and
R²⁴, R²⁵, R²⁶, and R²⁷ in each case independently of each other have the meaning of R¹, R² and R³;
f is 1 to 8;
g is 0 or I to (2f+1);
x is 0 to 3; and
h is 3 to 7;
including the physiologically active salts and prodrugs derived therefrom.

Exemplary compounds according to Formula I are described in European Patent Nos. EP0650960 and EP0650961. All compounds listed in EP0650960 and EP0650961, in particular, those listed in the compound claims and the final products of the working examples, are hereby incorporated into the present application by reference herein.

Additionally, exemplary compounds according to Formula I are described in U.S. Pat. No. 5,658,933. All compounds listed in U.S. Pat. No. 5,658,933, in particular, those listed in the compound claims and the final products of the working examples, are hereby incorporated into the present application by reference herein.

Additional compounds according to Formula I are substituted heterocyclic carboxamides, for example, those described in U.S. Pat. No. 5,620,995; 3-hydroxypyridine-2-carboxamidoesters described in U.S. Pat. No. 6,020,350; sulfonamidocarbonylpyridine-2-carboxamides described in U.S. Pat. No. 5,607,954; and sulfonamidocarbonyl-pyridine-2-carboxamides and sulfonamidocarbonyl-pyridine-2-carboxamide esters described in U.S. Pat. Nos. 5,610,172 and 5,620,996. All compounds listed in these patents, in particular, those compounds listed in the compound claims and the final products of the working examples, are hereby incorporated into the present application by reference herein.

Exemplary compounds according to Formula Ia are described in U.S. Pat. Nos. 5,719,164 and 5,726,305. All compounds listed in the foregoing patents, in particular, those listed in the compound claims and the final products of the working examples, are hereby incorporated into the present application by reference herein.

Exemplary compounds according to Formula Ib are described in U.S. Pat. No. 6,093,730. All compounds listed in U.S. Pat. No. 6,093,730, in particular, those listed in the compound claims and the final products of the working examples, are hereby incorporated into the present application by reference herein.

In particular embodiments, the compounds used in the methods and medicaments for reducing LDL-C, reducing VLDL-C, etc. in a subject in need thereof, are structural mimetics of 2-oxoglutarate, which may inhibit the target HIF prolyl hydroxylase enzyme competitively with respect to 2-oxoglutarate and noncompetitively with respect to iron. In another embodiment, compounds for use in the present methods and medicaments are heterocyclic carbonyl glycines of formula A:

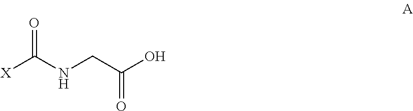

wherein X is an optionally substituted heterocyclic moiety.
   Such prolyl hydroxylase inhibitors include, but are not limited to, variously substituted 3-hydroxy-pyridine-2-carbonyl-glycines, 4-hydroxy-pyridazine-3-carbonyl-glycines, 3-hydroxy-quinoline-2-carbonyl-glycines, 4-hydroxy-2-oxo-1,2-dihydro-quinoline-3-carbonyl-glycines, 4-hydroxy-2-oxo-1,2-dihydro-naphthyridine-3-carbonyl-glycines, 8-hydroxy-6-oxo-4,6-dihydro-pyridopyrazine-7-carbonyl-glycines, 4-hydroxy-isoquinoline-3-carbonyl-glycines, 4-hydroxy-cinnoline-3-carbonyl-glycines, 7-hydroxy-thienopyridine-6-carbonyl-glycines, 4-hydroxy-thienopyridine-5-carbonyl-glycines, 7-hydroxy-thiazolopyridine-6-carbonyl-glycines, 4-hydroxy-thiazolopyridine-5-carbonyl-glycines, 7-hydroxy-pyrrolopyridine-6-carbonyl-glycines, 4-hydroxy-pyrrolopyridine-5-carbonyl-glycines, etc.

Additional suitable HIF hydroxylase inhibitors compounds are represented by formula II below and are described in U.S. Pat. Nos. 7,323,475; 7,629,357; 7,863,292; and 8,017,625, each of which patent is specifically incorporated herein by reference in their entireties.

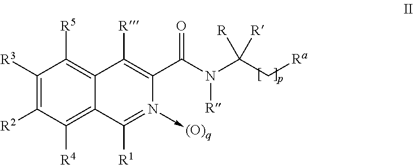

wherein:

q is zero or one;

p is zero or one;

R^{a} is —COOH or —WR⁸; provided that when R^{a} is —COOH then p is zero and when R^{a} is —WR⁸ then p is one;

W is selected from the group consisting of oxygen, —S(O)ₙ— and —NR⁹— where n is zero, one or two,

R⁹ is selected from the group consisting of hydrogen, alkyl, substituted alkyl, acyl, aryl, substituted aryl, hetemaryl, substituted heteroaryl, heterocyclic and substituted heterocyclic and R⁸ is selected from the group consisting of hydrogen, alkyl, substituted alkyl, aryl, substituted aryl, heteroaryl, substituted heteroaryl, heterocyclic and substituted heterocyclic, or when W is —NR⁹— then R¹ and R⁹, together with the nitrogen atom to which they are bound, can be joined to form a heterocyclic or a substituted heterocyclic group, provided that when W is —S(O)ₙ— and n is one or two, then R⁸ is not hydrogen;

R¹ is selected from the group consisting of hydrogen, alkyl, substituted alkyl, alkoxy, substituted alkoxy, amino, substituted amino, aminoacyl, aryl, substituted aryl, halo, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl or, when X is —NR⁷—, then R⁷ and R⁸, together with the nitrogen atom to which they are bound, can be joined to form a heterocyclic or substituted heterocyclic group;

R² and R³ are independently selected from the group consisting of hydrogen, alkyl, substituted alkyl, aryl, substituted aryl, heteroaryl, substituted heteroaryl, halo, hydroxy, cyano, —S(O)ₙ—N(R⁶)—R⁶ where n is 0, 1, or 2, —NR⁶C(O)NR⁶R⁶, —XR⁶ where X is oxygen, —S(O)ₙ— or —NR⁷— where n is zero, one or two, each R⁶ is independently selected from the group consisting of hydrogen, alkyl, substituted alkyl, aryl, substituted aryl, cycloalkyl, substituted cycloalkyl, heteroaryl, substituted heteroaryl, heterocyclic and substituted heterocyclic provided that when X is —SO— or —SO₂—, then R₆ is not hydrogen, and R⁷ is selected from the group consisting of hydrogen, alkyl, aryl, or R², R³ together with the carbon atom pendent thereto, form an aryl substituted aryl, heteroaryl, or substituted heteroaryl;

R⁴ and R⁵ are independently selected from the group consisting of hydrogen, halo, alkyl, substituted alkyl, alkoxy, substituted alkoxy, aryl, substituted aryl, heteroaryl, substituted heteroaryl and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl or, when X is —NR⁷—, then R⁷ and R⁸, together with the nitrogen atom to which they are bound, can be joined to form a heterocyclic or substituted heterocyclic group;

R is selected from the group consisting of hydrogen, deuterium and methyl;

R′ is selected from the group consisting of hydrogen, deuterium; alkyl and substituted alkyl; alternatively, R and R′ and the carbon pendent thereto can be joined to form cycloalkyl, substituted cycloalkyl, heterocyclic or substituted heterocyclic group;

R″ is selected from the group consisting of hydrogen and alkyl or R″ together with R′ and the nitrogen pendent thereto can be joined to form a heterocyclic or substituted heterocyclic group;

R′″ is selected from the group consisting of hydroxy, alkoxy, substituted alkoxy, acyloxy, cycloalkoxy, substituted cycloalkoxy, aryloxy, substituted aryloxy, hetemaryloxy, substituted heteroaryloxy, aryl, —S(O)ₙ—R¹⁰ wherein R¹⁰ is selected from the group consisting of alkyl, substituted alkyl, cycloalkyl, substituted cycloalkyl, aryl, substituted aryl, heteroaryl and substituted heteroaryl and n is zero, one or two;

and pharmaceutically acceptable salts, esters and prodrugs thereof.

Exemplary compounds of Formula II include, but are not limited to, {[4-Hydroxy-1-(naphthalen-2 yloxy)-isoquinoline-3-carbonyl]-amino}-acetic acid; {[4-Hydroxy-1-(pyridin-3 yloxy)-isoquinoline-3-carbonyl]-amino}-acetic acid; {[4-Hydroxy-1-(4-methoxy-phenoxy)-isoquinoline-3-carbonyl]-amino}-acetic acid; {[4-Hydroxy-1-(3-methoxy-phenoxy)-isoquinoline 3-carbonyl]-amino}-acetic acid; {[1-(3-Fluoro-phenoxy)-4-hydroxy-isoquinoline-3-carbonyl]-amino}-acetic acid; {[(1-(4-Fluoro-phenoxy)-4-hydroxy-isoquinoline-3-carbonyl]-amino}-acetic acid; {[1-(2-Fluoro-phenoxy)-4-hydroxy-isoquinoline-3-carbonyl]-amino}-acetic acid; {[4-Hydroxy-1-(2-methoxy-phenoxy)-isoquinoline-3-carbonyl]-amino}-acetic acid; {[1-(4-Acetylamino-phenoxy)-4-hydroxy-isoquinoline-3-carbonyl]-amino}-acetic acid; {[4-Hydroxy-1-(4-methanesulfonylamino-phenoxy)-isoquinoline-3-carbonyl]-amino}-acetic acid; [(4-Hydroxy-1-phenylamino-isoquinoline-3-carbonyl)-amino]-acetic acid; {[4-Hydroxy-6-(pyridin-3-yloxy)-isoquinoline-3-carbonyl]-amino}-acetic acid; {[4-Hydroxy-7-(pyridin-3-yloxy)-isoquinoline-3-carbonyl]-amino}-acetic acid; [(1-Chloro-4-methoxy-isoquinoline-3-carbonyl)-amino]-acetic acid; [(1-Chloro-4-ethoxy-isoquinoline-3-carbonyl)-amino]-acetic acid; [(4-Hydroxy-1-methoxy-isoquinoline-3-carbonyl)-amino]-acetic acid; [(1-Ethoxy-4-hydroxy-isoquinoline-3-carbonyl)-amino]-acetic acid; [(4-Acetoxy-1-phenyl-isoquinoline-3-carbonyl)-amino]-acetic acid; [(4-Hydroxy-1-phenyl-isoquinoline-3-carbonyl)-amino]-acetic acid; [(1-Ethoxy-4-phenyl-isoquinoline-3-carbonyl)-amino]-acetic acid; [(1-Chloro-4-phenyl-isoquinoline-3-carbonyl)-amino]-acetic acid; [(4-Phenyl-isoquinoline-3-carbonyl)-amino]-acetic acid; [(4-Hydroxy-t-methyl-isoquinoline-3-carbonyl)-amino]-acetic acid; [(4-Hydroxy-1-methoxymethyl-isoquinoline-3-carbonyl)-amino]-acetic acid; [(1-Dimethylcarbamoyl-4-hydroxy-isoquinoline-3-carbonyl)-amino]-acetic acid; [(4-Hydroxy-1-methyl-6-phenoxy-isoquinoline-3-carbonyl)-amino]-acetic acid; [(4-Hydroxy-1-methyl-7-phenoxy-isoquinoline-3-carbonyl)-amino]-acetic acid; [(4-Benzyloxy-1-methyl-7-phenoxy-isoquinoline-3-carbonyl)-amino]-acetic acid; [(4-Ethoxy-1-methyl-7-phenoxy-isoquinoline-3-carbonyl)-amino]-acetic acid; [(1-Dimethylcarbamnoyl-4-hydroxy-7-phenoxy-isoquinoline-3-carbonyl)-amino]-acetic acid; [(4-Hydroxy-1-methoxymethyl-7-phenoxy-isoquinoline-3-carbonyl)-amino]-acetic acid; [(4-Hydroxy-1-p-tolyl-isoquinoline-3-carbonyl)-amino]-acetic acid; {[7-(4-Fluoro-phenoxy)-4-hydroxy-1-methyl-isoquinoline-3-carbonyl]-amino}-acetic acid; {[1-Chloro-4-hydroxy-7-(4-methoxy-phenoxy)-isoquinoline-3-carbonyl]-amino}-acetic acid; {[4-Hydroxy-7-(4-methoxy-phenoxy)-isoquinoline-3-carbonyl]-amino}-acetic acid; {[1-Chloro-4-hydroxy-6-(4-methoxy-phenoxy)-isoquinoline-3-carbonyl]-amino}-acetic acid; {[4-Hydroxy-6-(4-methoxy-phenoxy)-isoquinoline-3-carbonyl]-amino}-acetic acid; {[1-Chloro-4-hydroxy-7-(4-trifluoromethyl-phenoxy)-isoquinaline-3-carbonyl]-amino}-acetic acid; {[4-Hydroxy-7-(4-trifluoromethyl-phenoxy)-isoquinoline-3-carbonyl]-amino}-acetic acid; {[1-Chloro-4-hydroxy-6-(4-trifluoromethyl-phenoxy)-isoquinoline-3-carbonyl]-amino}-acetic acid; {[4-Hydroxy-6-(4-trifluoromethyl-phenoxy)-isoquinoline-3-carbonyl]-amino}-acetic acid; {[1-Chloro-7-(4-fluoro-phenoxy)-4-hydroxy-isoquinoline-3-carbonyl]-amino}-acetic acid; {[7-(4-Fluoro-phenoxy)-4-hydroxy-isoquinoline-3-carbonyl]-amino}-acetic acid; {[1-Chloro-6-(4-fluoro-phenoxy)-4-hydroxy-isoquinoline-3-carbonyl]-amino}-acetic acid; {[6-(4-Fluoro-phenoxy)-4-hydroxy-isoquinoline-3-carbonyl]-amino}-acetic acid; {[4-Hydroxy-7-(pyridin-4-ylsulfanyl)-isoquinoline-3-carbonyl]-amino}-acetic acid; {[4-Hydroxy-6-(pyridin-4-ylsulfanyl)-isoquinoline-3-carbonyl]-amino}-acetic acid; [(7-Benzenesulfinyl-4-hydroxy-isoquinoline-3-carbonyl)-amino]-acetic acid; [(7-Benzenesulfonyl-4-hydroxy-isoquinoline-3-carbonyl)-amino]-acetic acid; [(6-Benzensulfinyl-4-hydroxy-isoquinaline-3-carbonyl)-amino]-acetic acid; [(6-Benzenesulfonyl-4-hydroxy-isoquinoline-3-carbonyl)-amino]-acetic acid; [(6-Amino-4-hydroxy-isoquinoline-3-carbonyl)-amino]-acetic acid; {[4-Hydroxy-7-(4-methoxy-benzenesulfonylamino)-isoquinoline-3-carbonyl]-amino}-acetic acid; {[4-Hydroxy-7-(3-phenyl-urido)-isoquinoline-3-carbonyl]-amino}-acetic acid; {[4-Hydroxy-6-(3-phenyl-ureido)-isoquinoline-3-carbonyl]-amino}-acetic acid; [(4-Hydroxy-1-phenylsulfanyl-isoquinoline-3-carbonyl)-amino]-acetic acid; {[1-(4-Chloro-phenylsulfanyl)-4-hydroxy-isoquinoline-3-carbonyl]-amino}-acetic acid; [(4-Hydroxy-1-p-tolylsulfanyl-isoquinoline-3-carbonyl)-amino]-acetic acid; {[4-Hydroxy-1-(pyridin-2-ylsulfanyl)-isoquinoline-3-carbonyl]-amino}-acetic acid; {[4-Hydroxy-1-(3-methoxy-phenylsulfanyl)-isoquinoline-3-carbonyl]-amino}-acetic acid; {[(4-Hydroxy-1-(2-methoxy-phenylsulfanyl)-isoquinoline-3-carbonyl]-amino}-acetic acid; {[4-Hydroxy-1-(naphthalen 2-ylsulfanyl)-isoquinoline-3-carbonyl]-amino}-acetic acid; [(1-Benzenesulfinyl-4-hydroxy-isoquinoline-3-carbonyl)-amino]-acetic acid; [(1-Benzenesulfonyl-4-hydroxy-isoquinoline-3-carbonyl)-amino]-acetic acid; {[4-Hydroxy-7-(pyridin-2-ylsulfanyl)-isoquinoline-3-carbonyl]-amino}-acetic acid; {([4-Hydroxy-6-(pyridin-2-ylsulfanyl)-isoquinoline-3-carbonyl]-amino}-acetic acid; [(1-Chloro-4-hydroxy-6,7-diphenoxy-isoquinoline-3-carbonyl)-amino]-acetic acid; [(4-Hydroxy-6,7-diphenoxy-isoquinoline-3-carbonyl)-amino]-acetic acid; ({4-Hydroxy-7-[4-(toluene-4-sulfonylamino)-phenoxy]-isoquinolin-3-carbonyl}-amino)-acetic acid; {[4-Hydroxy-7-(4-nitro-phenoxy)-isoquinoline-3-carbonyl]-amino}-acetic acid; [(4-Mercapto-7-phenoxy-isoquinoline-3-carbonyl)-amino]-acetic acid; [(4-Mercapto-7-trifluoromethyl-isoquinoline-3-carbonyl)-amino]-acetic acid; {[7-(4-Benzenesulfonylamino-phenoxy)-4-hydroxy-isoquinoline-3-carbonyl]-amino}-acetic acid; {[4-Hydroxy-7-(4-methanesulfonylamino-phenoxy)-isoquinoline-3-carbonyl]-amino}-acetic acid; {[7-(4-Chloro-phenoxy)-4-hydroxy-isoquinoline-3-carbonyl]-amino}-acetic acid; {[6-(4-Chloro-phenoxy)-4-hydroxy-isoquinoline-3-carbonyl]-amino}-acetic acid; {[6-(3-Fluoro-5 methoxy-phenoxy)-4-hydroxy-isoquinolin-3-carbonyl]-amino}-acetic acid; {[7-(3-Fluoro-5-methoxy-phenoxy)-4-hydroxy-isoquinoline-3-carbonyl]-amino}-acetic acid; {[7-(3,4-Difluoro-phenoxy)-4-hydroxy-isoquinoline-3-carbonyl]-amino}-acetic acid; {[6-(3,4-Difluoro-phenoxy)-4-hydroxy-isoquinoline-3-carbonyl]-amino}-acetic acid; {[4-Hydroxy-7-(4-trifluoromethoxy-phenoxy)-isoquinoline-3-carbonyl]-amino}-acetic acid; {[4-Hydroxy-6-(4-trifluoromethoxy-phenoxy)-isoquinolin-3-carbonyl-amino-acetic acid; 2-(S)-((7-(4-Chloro-phenoxy)-4-hydroxy-isoquinolin-3-carbonyl]-amino}-propionic acid; 2-(S)-{([6-(4-Chloro-phenoxy)-4-hydroxy-isoquinoline-3-carbonyl]-amino}-propionic acid; 2-{[7-(3,4-Difluoro-phenoxy)-4-hydroxy-isoquinoline-3-carbonyl]-amino}-propionic acid; 2-(S)-[(4-Hydroxy-7-phenylsulfanyl-isoquinoline-3-carbonyl)-amino]-propionic acid; 2-(R)-[(4-Hydroxy-7-phenylsulfanyl-isoquinoline-3-carbonyl)-amino]-propionic acid; 2-(R)-[(4-Hydroxy-7-phenoxy-isoquinoline-3-carbonyl)-amino]-propionic acid; 2-(S)-([4-Hydroxy-7-(4-methoxy-phenoxy)-isoquinoline-3-carbonyl]-amino)-propionic acid; 2-(S)-[(7-Benzenesulfonyl-4-hydroxy-isoquinoline-3-carbonyl)-amino]-propionic acid; (R)-2-[(4-Hydroxy-1-methoxymethyl-7-phenoxy-isoquinoline-3-carbonyl)-amino]-propionic acid; (S)-2-[(4-Hydroxy-1-methoxymethyl-7-phenoxy-isoquinoline-3-carbonyl)-amino]-propionic acid; (S)-2-[(4-Mercapto-7-phenoxy-isoquinoline-3-carbonyl)-amino]-propionic acid; (S)-2-{[1-(4-Chloro-phenylsulfanyl)-4-hydroxy-isoquinoline-3-carbonyl]-amino}-propionic acid; (R)-2-{[1-(4-Chloro-phenylsulfanyl)-4-hydroxy-isoquinoline-3-carbonyl]-amino}-propionic acid; [(4-Hydroxy-7-phenylsulfanyl-isoquinoline-3-carbonyl)-amino]-acetic acid; [(4-Hydroxy-6-phenylsulfanyl-isoquinoline-3-carbonyl)-amino]-acetic acid; [(1-Chloro-4-hydroxy-7-phenylsulfanyl-isoquinoline-3-carbonyl)-amino]-acetic acid; [(1-Chloro-4-hydroxy-6-phenylsulfanyl-isoquinoline-3-carbonyl)-amino]-acetic acid; [(1-Bromo-4-hydroxy-7-phenylsulfanyl-isoquinoline-3-carbonyl)-amino]-acetic acid; [(1-Bromo-4-hydroxy-6-phenylsulfanyl-isoquinoline-3-carbonyl)-amino]-acetic acid; [(4-Hydroxy-7-phenoxy-isoquinoline-3-carbonyl)-amino]-acetic acid; [(4-Hydroxy-6-phenoxy-isoquinoline-3-carbonyl)-amino]-acetic acid; [(1-Chloro-4-hydroxy-7-phenoxy-isoquinoline-3-carbonyl)-amino]-acetic acid; [(1-Chloro-4-hydroxy-6-phenoxy-isoquinoline-3-carbonyl)-amino]-acetic acid; [(1-Bromo-4-hydroxy-7-phenoxy-isoquinoline-3-carbonyl)-amino]-acetic acid; [(1-Bromo-4-hydroxy-6-phenoxy-isoquinoline-3-carbonyl)-amino]-acetic acid; {[7-(2,6-Dimethyl-phenoxy)-4-hydroxy-isoquinoline-3-carbonyl]-amino}-acetic acid; {[1-Chloro-7-(2,6-dimethyl-phenoxy)-4-hydroxy-isoquinoline-3-carbonyl]-amino}-acetic acid; {[1-Bromo-7-(2,6-dimethyl-phenoxy)-4-hydroxy-isoquinoline-3-carbonyl]-amino}-acetic acid; [(1-Bromo-7-chloro-4-hydroxy-isoquinoline-3-carbonyl)-amino]-acetic acid; [(1-Bromo-6-chloro-4-hydroxy-isoquinoline-3-carbonyl)-amino]-acetic acid; [(1-Bromo-4-hydroxy-7-trifluoromethyl-isoquinoline-3-carbonyl)-amino]-acetic acid; [(1-Bromo-4-hydroxy-6-trifluoromethyl-isoquinoline-3-carbonyl)-amino]-acetic acid; [(4-Hydroxy-1-phenoxy-isoquinoline-3-carbonyl)-amino]-acetic acid; [(1,7-dibromo-4-hydroxy-isoquinoline-3-carbonyl)-amino]-acetic acid; [(7-Bromo-)-chloro-4-hydroxy-isoquinoline-3-carbonyl)-amino]-acetic acid; [(6-Bromo-4-hydroxy-isoquinolin-3-carbonyl)-amino]-acetic acid; [(1-Bromo-7-fluoro-4-hydroxy-isoquinoline-3-carbonyl)-amino]-acetic acid; [(7-Fluoro-4-hydroxy-isoquinoline-3-carbonyl)-amino]-acetic acid; [(1-Chloro-7-fluoro-4-hydroxy-isoquinoline-3-carbonyl)-amino]-acetic acid; [(1-Chloro-4-hydroxy-benzo[g]isoquinoline-3-carbonyl)-amino]-acetic acid; [(1-Bromo-4-hydroxy-isoquinoline-3-carbonyl)-amino]-acetic acid; [(4-Hydroxy-6-phenyl-isoquinoline-3-carbonyl)-amino]-acetic acid; [(4-Hydroxy-7-phenyl-isoquinoline-3-carbonyl)-amino]-acetic acid; [(1-Chloro-4-hydroxy-6-phenyl-isoquinoline-3-carbonyl)-amino]-acetic acid; [(1-Chloro-4-hydroxy-7-phenyl-isoquinoline-3-carbonyl)-amino]-acetic acid; [(1-Bromo-4-hydroxy-6-phenyl-isoquinoline-3-carbonyl)-amino]-acetic acid; [(1-Bromo-4-hydroxy-7-phenyl-isoquinoline-3-carbonyl)-amino]-acetic acid; [(4-Hydroxy-5-phenyl-isoquinoline-3-carbonyl)-amino]-acetic acid; [(4-Hydroxy-8-phenyl-isoquinoline-3-carbonyl)-amino]-acetic acid; [(1-Chloro-4-hydroxy-5-phenyl-isoquinoline-3-carbonyl)-amino]-acetic acid; [(1-Chloro-4-hydroxy-8-phenyl-isoquinoline-3-carbonyl)-amino]-acetic acid; [(1-Bromo-4-hydroxy-5-phenyl-isoquinoline-3-carbonyl)-amino]-acetic acid; [(1-Bromo-4-hydroxy-8-phenyl-isoquinoline-3-carbonyl)-amino]-acetic acid; [(1-Ethylsulfanyl-4-hydroxy-isoquinoline-3-carbonyl)-amino]-acetic acid; {[4-Hydroxy-1-(4-methoxy-phenylsulfanyl)-isoquinoline-3-carbonyl]-amino}-acetic acid; [(1-Chloro-4-hydroxy-7-iodo-isoquinoline-3-carbonyl)-amino]-acetic acid; [(1-Chloro-4-hydroxy-6-iodo-isoquinoline-3-carbonyl)-amino]-acetic acid; [(4-Hydroxy-7-iodo-isoquinoline-3-carbonyl)-amino]-acetic acid; [(1-Bromo-4-hydroxy-7-methyl-isoquinoline-3-carbonyl)-amino]-acetic acid; [(1-Bromo-7-butoxy-4-hydroxy-isoquinoline-3-carbonyl)-amino]-acetic acid; [(1-Bromo-6-butoxy-4-hydroxy-isoquinoline-3-carbonyl)-amino]-acetic acid; [(6-Benzyloxy-1-chloro-4-hydroxy-isoquinoline-3-carbonyl)-methyl-amino]-acetic acid; [(1-Chloro-4-hydroxy-isoquinoline-3-carbonyl)-methyl-amino]-acetic acid; [(1-Chloro-4-hydroxy-6-isopropoxy-isoquinoline-3-carbonyl)-methyl-amino]-acetic acid; [(1-Chloro-4-hydroxy-7-isopropoxy-isoquinoline-3-carbonyl)-methyl-amino]-acetic acid; [Carboxymethyl-(1-chloro-4-hydroxy-isoquinoline-3-carbonyl)-amino]-acetic acid; [Carboxymethyl-(1-chloro-4-hydroxy-6-isopropoxy-isoquinoline-3-carbonyl)-amino]-acetic acid; 1-Chloro-4-hydroxy-isoquinoline-3-carboxylic acid (2-amino-ethyl)-amide (trifluoro-acetic acid salt); 1-Chloro-4-hydroxy-isoquinoline-3-carboxylic acid (2-methoxy-ethyl)-amide; 1-Chloro-4-hydroxy-isoquinoline-3-carboxylic acid (2-hydroxy-ethyl)-amide; 1-Chloro-4-hydroxy-isoquinoline-3-carboxylic acid (2-dimethylamino-ethyl)-amide; 1-Chloro-hydroxy-isoquinoline-3-carboxylic acid (2-acetylamino-ethyl)-amide; 1-Chloro-4-hydroxy-6-isopropoxy-isoquinoline-3-carboxylic acid (2-hydroxy-ethyl)-amide; 1-Chloro-4-hydroxy-6-isopropoxy-isoquinoline-3-carboxylic acid (2-methoxy-ethyl)-amide; 1-Chloro-4-hydroxy-6-isopropoxy-isoquinoline-3-carboxylic acid (2-amino-ethyl)-amide (trifluoro-acetic acid salt); 1-Chloro-4-hydroxy-6-isopropoxy-isoquinoline-3-carboxylic acid (2-dimethylamino-ethyl)-amide; 1-Chloro-4-hydroxy-7-isopropoxy-isoquinoline-3-carboxylic acid (2-amino-ethyl)-amide (trifluoro-acetic acid salt); 1-Chloro-4-hydroxy-7-isopropoxy-isoquinoline-3-carboxylic acid (2-methoxy-ethyl)-amide; 1-Chloro-4-hydroxy-7-isopropoxy-isoquinoline-3-carboxylic acid (2-dimethylamino-ethyl)-amide; 1-Chloro-4-hydroxy-7-isopropoxy-isoquinoline-3-carboxylic acid (2-hydroxy-ethyl)-amide; (S)-2-[(6-Benzyloxy-1-chloro-4-hydroxy-isoquinoline-3-carbonyl)-amino]-propionic acid; (R)-2-[(1-Chloro-4-hydroxy-isoquinoline-3-carbonyl)-amino]-3-hydroxy-propionic acid; (S)-2-[(1-Chloro-4-hydroxy-isoquinoline-3-carbonyl)-amino]-3-hydroxy-propionic acid; (R)-2-[(1-Chloro-4-hydroxy-6-isopropoxy-isoquinolin-3-carbonyl)-amino]-3-hydroxy-propionic acid; (S)-2-[(1-Chloro-4-hydroxy-6-isopropoxy-isoquinoline-3-carbonyl)-amino]-3-hydroxy-propionic acid; (R)-2-[(1-Chloro-4-hydroxy-7-isopropoxy-isoquinoline-3-carbonyl)-amino]-3-hydroxy-propionic acid; (S)-2-[(1-Chloro-4-hydroxy-7-isopropoxy-isoquinoline-3-carbonyl)-amino]-3-hydroxy-propionic acid; 2-[(1-Chloro-4-hydroxy-isoquinoline-3-carbonyl)-amino]-2-methyl-propionic acid; 2-[(1-Chloro-4-hydroxy-6-isopropoxy-isoquinoline-3-carbonyl)-amino]-2-methyl-propionic acid; (R)-2-[(1-Chloro-4-hydroxy-isoquinoline-3-carbonyl)-amino]-3-(1H-imidazol-4-yl)-propionic acid (trifluoro-acetic acid salt); (S)-2-[(1-Chloro-4-hydroxy-isoquinoline-3-carbonyl)-amino]-3-(1H-imidazol-4-yl)-propionic acid (trifluoro-acetic acid salt); (R)-2-[(1-Chloro-4-hydroxy-isoquinoline-3-carbonyl)-amino]-3-methyl-butyric acid; (S)-2-[(1-Chloro-4-hydroxy-isoquinoline-3-carbonyl)-amino]-3-methyl-butyric acid; (R)-2-[(1-Chloro-4-hydroxy-6-isopropoxy-isoquinoline-3-carbonyl)-amino]-3-methyl-butyric acid; (S)-2-[(1-Chloro-4-hydroxy-6-isopropoxy-isoquinoline-3-carbonyl)-amino]-3-methyl-butyric acid; (R)-2-[(1-Chloro-4-hydroxy-7-isopropoxy-isoquinoline-3-carbonyl)-amino]-3-methyl-butyric acid; (S)-2-[(1-Chloro-4-hydroxy-7-isopropoxy-isoquinoline-3-carbonyl)-amino]-3-methyl-butyric acid; (S)-2-[(6-Benzyloxy-1-chloro-4-hydroxy-isoquinoline-3-carbonyl)-amino]-3-methyl-butyric acid; (R)-2-[(1-Chloro-4-hydroxy-isoquinoline-3-carbonyl)-amino]-3-phenyl-propionic acid; (S)-2-[(1-Chloro-4-hydroxy-isoquinoline-3-carbonyl)-amino]-3-phenyl-propionic acid; (R)-2-[(1-Chloro-4-hydroxy-6-isopropoxy-isoquinoline-3-carbonyl)-amino]-3-phenyl-propionic acid; (S)-[(1-Chloro-4-hydroxy-6-isopropoxy-isoquinoline-3-carbonyl)-amino]-3-phenyl-propionic acid; (R)-2-[(1-Chloro-4-hydroxy-7-isopropoxy-isoquinoline-3-carbonyl)-amino]-3-phenyl-propionic acid; (S)-2-[(1-Chloro-4-hydroxy-7-isopropoxy-isoquinoline-3-carbonyl)-amino]-3-phenyl-propionic acid; (R)-2-[(1-Chloro-4-hydroxy-isoquinoline-3-carbonyl)-amino]-3-(4-hydroxy-phenyl)-propionic acid; (S)-2-[(1-Chloro-4-hydroxy-isoquinoline-3-carbonyl)-amino]-3-(4-hydroxy-phenyl)-propionic acid; (R)-2-[(1-Chloro-4-hydroxy-6-isopropoxy-isoquinoline-3-carbonyl)-amino]-3-(4-hydroxy-phenyl)-propionic acid; (S)-2-[(1-Chloro-4-hydroxy-6-isopropoxy-isoquinoline-3-carbonyl)-amino]-3-(4-hydroxy-phenyl)-propionic acid; (R)-2-[(1-Chloro-4-hydroxy-7-isopropoxy-isoquinoline-3-carbonyl)-amino-3-(4-hydroxy phenyl)-propionic acid; (S)-2-(1-Chlor-4-hydroxy-7-isopropoxy-isoquinoline-3-carbonyl)-amino]-3-(4-hydroxy-phenyl)-propionic acid; (R)-2-[(1-Chloro-4-hydroxy-6-isopropoxy-isoquinoline-3-carbonyl)-amino]-pentanoic acid; (S)-2-[(1-Chloro-4-hydroxy-6-isopropoxy-isoquinoline-3-carbonyl)-amino]-pentanoic acid; (R)-1-[(1-Chloro-4-hydroxy-isoquinoline-3-carbonyl)-pyrrolidine-2-carboxylic acid; (S)-1-(1-Chloro-4-hydroxy-isoquinoline-3-carbonyl)-pyrrolidine-2-carboxylic acid; (R)-1-(1-Chloro-4-hydroxy-6-isopropoxy-isoquinoline-3-carbonyl)-pyrrolidine-2-carboxylic acid; (S)-1-(1-Chloro-4-hydroxy-6-isopropoxy-isoquinoline-3-carbonyl)-pyrrolidine-2-carboxylic acid; (R)-6-Amino-2-[(1-chloro-4-hydroxy-isoquinoline-3-carbonyl)-amino]-hexanoic acid (trifluoro-acetic acid salt); (S)-6-Amino-2-[(1-chloro-4-hydroxy-isoquinoline-3-carbonyl)-amino]-hexanoic acid (trifluoro-acetic acid salt); (R)-6-Amino-2-[(1-chloro-4-hydroxy-6-isopropoxy-isoquinoline-3-carbonyl)-amino]-hexanoic acid; trifluoroacetic acid salt; (S)-6-Amino-2-[(1-chloro-4-hydroxy-6-isopropoxy-isoquinoline-3-carbonyl)-amino]-hexanoic acid (trifluoro-acetic acid salt); (R)-6-Amino-2-[(1-chloro-4-hydroxy-7-isopropoxy-isoquinoline-3-carbonyl)-amino]-hexanoic acid; trifluoroacetic acid salt; (S)-6-Amino-2-[(1-chloro-4-hydroxy-7-isopropoxy-isoquinoline 3-carbonyl)-amino]-hexanoic acid (trifluoro-acetic acid salt); (R)-2-[(1-Chloro-4-hydroxy-isoquinoline-3-carbonyl)-amino]-succinic acid; (S)-2-[(1-Chloro-4-hydroxy-isoquinoline-3-carbonyl)-amino]-succinic acid; (R)-2-[(1-Chloro-4-hydroxy-6-isopropoxy-isoquinoline-3-carbonyl)-amino]-succinic acid; (S)-2-[(1-Chloro-4-hydroxy-6-isopropoxy-isoquinoline-3-carbonyl)-amino]-succinic acid; (R)-2-[(1-Chloro-4-hydroxy-7-isopropoxy-isoquinoline-3-carbonyl)-amino]-succinic acid; 1-[(1-Chloro-4-hydroxy-isoquinoline-3-carbonyl)-amino]-cyclopropanecarboxylic acid; 1-[(1-Chloro-4-hydroxy-6-isopropoxy-isoquinoline-3-carbonyl)-amino]-cyclopropanecarboxylic acid; Dideutero-[(1-chloro-4-hydroxy-isoquinoline-3-carbonyl)-amino]-acetic acid; (R)-2-[(6-Benzyloxy-1-chloro-4-hydroxy-isoquinoline-3-carbonyl)-amino]-propionic acid; (S)-2-[(7-Benzyloxy-1-chloro-4-hydroxy-isoquinoline-3-carbonyl)-amino]-propionic acid; (R)-2-[(7-Benzyloxy-1-chloro-4-hydroxy-isoquinoline-3-carbonyl)-amino]-propionic acid; (S)-2-[(1-Chloro-4-hydroxy-isoquinoline-3-carbonyl)-amino]-propionic acid; (R)-2-[(1-Chloro-4-hydroxy-isoquinoline-3-carbonyl)-amino]-propionic acid; (S)-2-[(6-Isopropoxy-1-chloro-4-hydroxy-isoquinoline-3-carbonyl)-amino]-propionic acid; (R)-2-[(6-Isopropoxy-1-chloro-4-hydroxy-isoquinoline-3-carbonyl)-amino]-propionic acid; (S)-2-[(7-Isopropoxy-1-chloro-4-hydroxy-isoquinoline-3-carbonyl)-amino]-propionic acid; (R)-2-[(7-Isopropoxy-1-chloro-4-hydroxy-isoquinoline-3-carbonyl)-amino]propionic acid; 1-Chloro-4-hydroxy-6-isopropoxy-isoquinoline-3-carboxylic acid (2-hydroxy-1-hydroxymethyl-ethyl)-amide; 1-Chloro-4-hydroxy-7-isopropoxy-isoquinoline-3-carboxylic acid (2-hydroxy-1-hydroxymethyl-ethyl)-amide; 1-Chloro-4-hydroxy-isoquinoline-3-carboxylic acid (2-hydroxy-1-hydroxymethyl-ethyl)-amide; {[7-(3,5-Difluoro-phenoxy)-4-hydroxy-isoquinoline-3-carbonyl]-amino}-acetic acid; {([6-(3,5-Difluoro-phenoxy)-4-hydroxy-isoquinoline-3-carbonyl]-amino}-acetic acid; ({7-[(4-(4-Fluoro-phenoxy)-phenoxy]-4-hydroxy-isoquinolin-3-carbonyl)-amino}-acetic acid; ({6-[4-(4-Fluoro-phenoxy)-phenoxy]-4-hydroxy-isoquinoline-3-carbonyl}-amino)-acetic acid; {[7-(3-Chloro-4-fluoro-phenoxy)-4-hydroxy-isoquinoline-3-carbonyl]-amino}-acetic acid; {[6-(3-Chloro-4-fluoro-phenoxy)-4-hydroxy-isoquinoline-3-carbonyl]-amino}-acetic acid; (S)-2-{[7-(3-Fluoro-5-methoxy-phenoxy)-4-hydroxy-isoquinoline-3-carbonyl]-amino}-propionic acid; 2-(S)-[(7-Cyclohexyloxy-4-hydroxy-isoquinoline-3-carbonyl)-amino]-propionic acid; 2-(S)-{[7-(4-Fluoro-phenoxy)-4-hydroxy-1-methyl-isoquinoline-3-carbonyl]-amino}-propionic acid; 2-(S)-{[7-(4-Fluoro-phenoxy)-4-hydroxy-isoquinoline-3-carbonyl]-amino}-propionic acid; 2-(S)-{[7-(4-Hydroxy-1-methyl-7-phenoxy-isoquinoline-3-carbonyl]-amino}-propionic acid; 2-(S)-[(4-Hydroxy-1-methyl-7-phenylsulfanyl-isoquinoline-3-carbonyl)-amino]-propionic acid; 2-(S)-[4-Hydroxy-7-(4-trifluoromethyl-phenoxy)-isoquinoline-3-carbonyl]-amino)-propionic acid; {[7-(4-Chloro-phenoxy)-4-hydroxy-1-methyl-isoquinoline-3-carbonyl]-amino}-acetic acid; {[6-(4-Chloro-phenoxy)-4-hydroxy-1-methyl-isoquinoline-3-carbonyl]-amino}-acetic acid; {[7-(3,5-Difluoro-phenoxy)-4-hydroxy-1-methyl-isoquinoline-3-carbonyl]-amino}-acetic acid; {[4-Hydroxy-7-(4-methoxy-phenoxy)-1-methyl-isoquinoline-3-carbonyl]-amino}-acetic acid; {[4-Hydroxy-6-(4-methoxy-phenoxy)-1-methyl-isoquinoline-3-carbonyl]-amino}-acetic acid; [(6-Cyclohexyloxy-4-hydroxy-isoquinoline-3-carbonyl)-amino]-acetic acid; [(7-Cyclohexyloxy-4-hydroxy-isoquinoline-3-carbonyl)-amino]-acetic acid; [(7-Cyclohexyloxy-4-hydroxy-1-methyl-isoquinoline-3-carbonyl)-amino]-acetic acid; [(7-Cyclohexylsulfanyi-4-hydroxy-isoquinoline-3-carbonyl)-amino]-acetic acid; [(7-Cyclohexanesulfonyl-4-hydroxy-isoquinoline-3-carbonyl)-amino]-acetic acid; [(4-Hydroxy-1-isobutyl-isoquinoline-3-carbonyl)-amino]-acetic acid; [(4-Hydroxy-1-pyridin-2-yl-isoquinoline-3-carbonyl)-amino]-acetic acid; [(1-Ethyl-4-hydroxy-7-phenoxy-isoquinoline-3-carbonyl)-amino]-acetic acid; [(1-Dimethylaminomethyl-4-hydroxy-7-phenylsulfanyl-isoquinoline-3-carbonyl)-amino]-acetic acid; [(4-Hydroxy-1-methyl-7-phenylsulfanyl-isoquinoline-3-carbonyl)-amino]-acetic acid; and {[4-Hydroxy-1-methyl-7-(4-trifluoromethyl-phenoxy)-isoquinoline-3-carbonyl]-amino}-acetic acid.

Additional suitable HIF hydroxylase inhibitor compounds are represented by formula III below and are described in U.S. Pat. No. 7,928,120, which patent is specifically incorporated herein by reference in its entirety.

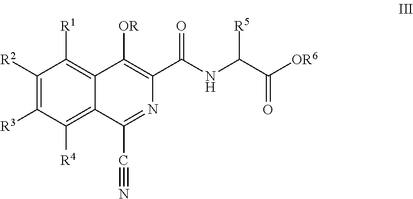

wherein:

R is selected from the group consisting of hydrogen, alkyl, and substituted alkyl;

R¹, R², R³ and R⁴ are independently selected from the group consisting of hydrogen, halo, cyano, hydroxyl, alkyl, substituted alkyl, aryl, substituted aryl, heteroaryl, substituted heteroaryl, amino, substituted amino, —OR⁷, —SR⁷, —SOR⁷, and —SO₂R⁷ wherein R⁷ is selected from the group consisting of alkyl, substituted alkyl, cycloalkyl, substituted cycloalkyl, aryl, substituted aryl, heteroaryl, and substituted heteroaryl; and

R⁵ and R⁶ are independently selected from the group consisting of hydrogen or C₁₋₃ alkyl;

or pharmaceutically acceptable salts, tautomers, stereoisomers, solvates, and/or prodrugs thereof.

Exemplary compounds of Formula III include, without limitation, {[1-cyano-4-hydroxy-isoquinoline-3-carbonyl]-amino}-acetic acid, 2-(S)-[(1-cyano-4-hydroxy-isoquinoline-3-carbonyl)-amino]-propionic acid, {[1-cyano-4-hydroxy-7-phenoxy-isoquinoline-3-carbonyl]-amino}-acetic acid, 2-(S)-[(1-cyano-4-hydroxy-7-phenoxy-isoquinoline-3-carbonyl)-amino]-propionic acid, 2-(R)-[(1-cyano-4-hydroxy-7-phenoxy-isoquinoline-3-carbonyl)-amino]-propionic acid, {[1-cyano-7-(4-fluorophenoxy)-4-hydroxy-isoquinoline-3-carbonyl]-amino}-acetic acid, {[1-cyano-4-hydroxy-7-(trifluoromethyl)-isoquinoline-3-carbonyl]-amino}-acetic acid, {[1-cyano-4-hydroxy-7-chloro-isoquinoline-3-carbonyl]-amino}-acetic acid, {[1-cyano-4-hydroxy-8-phenoxy-isoquinoline-3-carbonyl]-amino}-acetic acid, {[1-cyano-8-(4-fluoro-phenoxy)-4-hydroxy-isoquinoline-3-carbonyl]-amino}-acetic acid, [(1-cyano-4-hydroxy-6-methoxy-isoquinoline-3-carbonyl)-amino]-acetic acid, [(1-cyano-4-hydroxy-6-phenoxy-isoquinoline-3-carbonyl)-amino]-acetic acid, {[1-cyano-6-(4-fluoro-phenoxy)-4-hydroxy-isoquinoline-3-carbonyl]-amino}-acetic acid, {[1-cyano-4-hydroxy-6-(4-methoxy-phenoxy)-isoquinoline-3-carbonyl]-amino}-acetic acid, [(1-cyano-4-hydroxy-6-phenylsulfanyl-isoquinoline-3-carbonyl)-amino]-acetic acid, [(1-cyano-4-hydroxy-7-phenylsulfanyl-isoquinoline-3-carbonyl)-amino]-acetic acid, {[1-cyano-6-(2,6-dimethyl-phenoxy)-4-hydroxy-isoquinoline-3-carbonyl]-amino}-acetic acid, [(1-cyano-4-hydroxy-5-phenoxy-isoquinoline-3-carbonyl)-amino]-acetic acid, {[1-cyano-4-hydroxy-8-(4-methoxy-phenoxy)-isoquinoline-3-carbonyl]-amino}-acetic acid, {[1-cyano-4-hydroxy-8-(3-methoxy-phenoxy)-isoquinoline-3-carbonyl]-amino}-acetic acid, {[1-cyano-4-hydroxy-8-(2-methoxy-phenoxy)-isoquinoline-3-carbonyl]-amino}-acetic acid, [(7-benzyl-1-cyano-4-hydroxy-isoquinoline-3-carbonyl)-amino]-acetic acid, {[1-cyano-5-(4-fluoro-phenoxy)-4-hydroxy-isoquinoline-3-carbonyl]-amino}-acetic acid, {[1-cyano-7-(2,6-dimethyl-phenoxy)-4-hydroxy-isoquinoline-3-carbonyl]-amino}-acetic acid, {[1-cyano-6-(2-ethyl-6-methyl-phenoxy)-4-hydroxy-isoquinoline-3-carbonyl]-amino}-acetic acid, {[1-cyano-4-hydroxy-6-(2,4,6-trimethyl-phenoxy)-isoquinoline-3-carbonyl]-amino}-acetic acid, {[6-(4-chloro-2,6-dimethyl-phenoxy)-1-cyano-4-hydroxy-isoquinoline-3-carbonyl]-amino}-acetic acid, {[1-cyano-4-hydroxy-7-(4-methoxy-phenoxy)-isoquinoline-3-carbonyl]-amino}-acetic acid, [(1-cyano-6-cyclohexyloxy-4-hydroxy-isoquinolin-3-carbonyl)-amino]-acetic acid, [(6-benzenesulfonyl-1-cyano-4-hydroxy-isoquinoline-3-carbonyl)-amino]-acetic acid, {[1-cyano-4-hydroxy-6-(4-propoxy-phenoxy)-isoquinoline-3-carbonyl]-amino}-acetic acid, {([7-(benzo[1,3]dioxol-5-yloxy)-1-cyano-4-hydroxy-isoquinoline-3-carbonyl]-amino}-acetic acid, {[6-(benzo[1,3]dioxol-5-yloxy)-1-cyano-4-hydroxy-isoquinoline-3-carbonyl]-amino}-acetic acid, {[1-cyano-6-(2,3-dihydro-benzofuran-5-yloxy)-4-hydroxy-isoquinoline-3-carbonyl]-amino}-acetic acid, [(1-cyano-4-methoxy-8-phenoxy-isoquinoline-3-carbonyl)-amino]-acetic acid methyl ester, [(1-cyano-4-methoxy-8-phenoxy-isoquinoline-3-carbonyl)-amino]-acetic acid, (S)-2-[(1-cyano-4-hydroxy-8-phenoxy-isoquinoline-3-carbonyl)-amino]-propionic acid, (R)-2-[(1-cyano-4-hydroxy-8-phenoxy-isoquinoline-3-carbonyl)-amino]-propionic acid, {[1-cyano-4-hydroxy-6-(2-methyl-benzothiazol-6-yloxy)-isoquinoline-3-carbonyl]-amino}-acetic acid, {[1-cyano-6-(2-dimethylamino-benzooxazol-5-yloxy)-4-hydroxy-isoquinoline-3-carbonyl]-amino}-acetic acid, {[1-cyano-7-(2-dimethylamino-benzooxazol-S yloxy)-4-hydroxy-isoquinoline-3-carbonyl]-amino}-acetic acid, {[1-cyano-4-hydroxy-6-(2-morphoin-4-yl-benzothazol-6-yloxy)-isoquinoline-3-carbonyl]-amino}-acetic acid, {[1-cyano-4-hydroxy-6-(2-methyl-benzooxazol-6-yloxy)-isoquinoline-3-carbonyl]-amino}-acetic acid, [(6-chloro-1-cyano-4-hydroxy-isoquinoline-3-carbonyl)-amino]-acetic acid, [(7-butoxy-1-cyano-4-hydroxy-isoquinoline-3-carbonyl)-amino]-acetic acid, [(1-cyano-4-hydroxy-6,7-diphenoxy-isoquinoline-3-carbonyl)-amino]-acetic acid, [(1-cyano-4-hydroxy-7-methoxy-isoquinoline-3-carbonyl)-amino]-acetic acid, [(1-cyano-4-hydroxy-7-isopropoxy-isoquinoline-3-carbonyl)-amino]-acetic acid, [(1-cyano-4-hydroxy-6-isopropoxy-isoquinoline-3-carbonyl)-amino]-acetic acid, [(1-cyano-4-hydroxy-5-phenyl-isoquinoline-3-carbonyl)-amino]-acetic acid, [(1-cyano-4-hydroxy-8-phenyl-isoquinoline-3-carbonyl)-amino]-acetic acid, [(7-benzyloxy-1-cyano-4-hydroxy-isoquinoline-3-carbonyl)-amino]-acetic acid, {[5-(4-Chloro-phenoxy)-1-cyano-4-hydroxy-isoquinoline-3-carbonyl]-amino}-acetic acid, and [(1-cyano-4,7-dihydroxy-isoquinoline-3-carbonyl)-amino]-acetic acid.

Additional suitable HIF hydroxylase inhibitors compounds are represented by formula IV below and are described in U.S. Pat. No. 7,696,223, which patent is specifically incorporated herein by reference in its entirety.

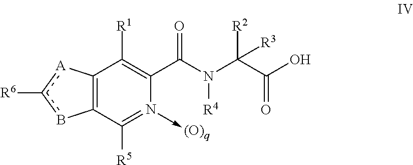

wherein:
q is 0 or 1;
A and B are independently selected from the group consisting —C(R⁷)—, —N(R⁸)—, ═N—, and —S— with the proviso that one of the following is present:
   A is ═C(R⁷)— and B is —N(R⁸)—;
   A is —S— and B is —N—;
   A ═N— and B is —S—; or
   A is —N(R⁸)— and B is ═C(R⁷)—;
one of -A [symbol] C(R⁶)— or —B [symbol] C(R⁶)— is a double bond and the other is a single bond;
R¹ is selected from the group consisting of hydroxyl, alkoxy, substituted alkoxy, acyloxy, cycloalkoxy, substituted cycloalkoxy, aryloxy, substituted aryloxy, heteroaryloxy, substituted heteroaryloxy, heterocyclyloxy, substituted heterocyclyloxy, mercapto, thioether, substituted alkylthio, arylsulfanyl, heteroarylsulfanyl, amino, substituted amino, acylamino and aminoacyl;
R² is selected from the group consisting of hydrogen, deuterium, and methyl;
R³ is selected from the group consisting of hydrogen, deuterium, alkyl, and substituted alkyl;
R⁴ is selected from the group consisting of hydrogen, alkyl, and substituted alkyl;
R⁵ is selected from the group consisting of hydrogen, halo, cyano, hydroxyl, alkyl, substituted alkyl, cycloalkyl, substituted cycloalkyl, alkenyl, substituted alkenyl, alkynyl, substituted alkynyl, alkoxy, substituted alkoxy, cycloalkoxy, substituted cycloalkoxy, aryl, substituted aryl, aryloxy, substituted aryloxy, heteroaryl, substituted heteroaryl, heterocyclyl, substituted heterocyclyl, heterocyclyloxy, substituted heterocyclyloxy, heteroaryloxy, substituted heteroaryloxy, acyl, aminoacyl, nitro, amino, substituted amino, acylamino, sulfanyl, sulfonyl, thioether, arylthio, and substituted arylthio;
R⁶ and R⁷ are each independently selected from the group consisting of hydrogen, halo, cyano, hydroxyl, alkyl, substituted alkyl, cycloalkyl, substituted cycloalkyl, alkenyl, substituted alkenyl, alkynyl, substituted alkynyl, alkoxy, substituted alkoxy, cycloalkoxy, substituted cycloalkoxy, aryl, substituted aryl, aryloxy, substituted aryloxy, heteroaryl, substituted heteroaryl, heterocyclyl, substituted heterocyclyl, heterocyclyloxy, substituted heterocyclyloxy, heteroaryloxy, substituted heteroaryloxy, acyl, aminoacyl, nitro, amino, substituted amino, acylamino, sulfanyl, sulfonyl, thioether, arylthio, and substituted arylthio;
or where when A or B is ═C(R⁷)—, then R⁶ and R⁷ together with the carbon atoms bound thereto join to form a cycloalkenyl, substituted cycloalkenyl, aryl, substituted aryl, heteroaryl, or substituted heteroaryl; and
R⁸ is selected from the group consisting of hydrogen, hydroxyl, alkyl, substituted alkyl, alkenyl, substituted alkenyl, alkynyl, substituted alkynyl, aryl, substituted aryl, heteroaryl, and substituted heteroaryl;
or pharmaceutically acceptable salts, single stereoisomers, mixtures of stereoisomers, esters, or prodrugs thereof.

Exemplary compounds of formula IV include, but are not limited to, [(2-bromo-4-hydroxy-1-phenyl-1H-pyrrolo[2,3-c]pyridine-5-carbonyl)-amino]-acetic acid, [(4-hydroxy-1-phenyl-1H-pyrrolo[2,3-c]pyridine-5-carbonyl)-amino]-acetic acid, [(2,3-dibromo-4-hydroxy-1-phenyl-1H-pyrrolo[2,3-c]pyridine-5-carbonyl)-amino]-acetic acid, {[3-bromo-2-(4-fluoro-phenyl)-4-hydroxy-1-phenyl-1H-pyrrolo[2,3-c]pyridine-5-carbonyl]-amino}-acetic acid, [(1-benzyl-2,3,-dibromo-4-hydroxy-1H-pyrrolo[2,3-c]pyridine-5-carbonyl]-amino}-acetic acid, {[2-(4-fluoro-phenyl)-4-hydroxy-1-phenyl-1H-pyrrolo[2,3-c]pyridine-5-carbonyl]-amino}-acetic acid, [(1-benzyl-4-hydroxy-1H-pyrrolo[2,3-c]pyridine-5-carbonyl)-amino]-acetic acid, {[3-bromo-1,2-bis-(4-fluoro-phenyl)-4-hydroxy-1H-pyrrolo[2,3-c]pyridine-5-carbonyl]-amino}-acetic acid, {[1,2-bis-(4-fluoro-phenyl)-4-hydroxy-1H-pyrrolo[2,3-c]pyridine-5-carbonyl]-amino}-acetic acid, {[3-chloro-1,2-bis-(4-fluoro-phenyl)-4-hydroxy-1H-pyrrolo[2,3-c]pyridine-5-carbonyl]-amino}-acetic acid, {[3-bromo-2-(4-fluoro-phenyl)-4-hydroxy-1-(4-methoxy-phenyl)-1H-pyrrolo[2,3-c]pyridine-5-carbonyl]-amino}-acetic acid, {[2-(4-fluoro-phenyl)-4-hydroxy-1-(4-methoxy-phenyl)-1H-pyrrolo[2,3-c]pyridine-5-carbonyl]-amino}-acetic acid, {[2-bromo-1-(4-fluoro-phenyl)-4 hydroxy-3-phenyl-1H-pyrrolo[2,3-c]pyridine-5-carbonyl]-amino}-acetic acid, {[1-(4-fluoro-phenyl)-4-hydroxy-3-phenyl-1H-pyrrolo[2,3-c]pyridine-5-carbonyl]-amino}-acetic acid, {[7-chloro-1-(4-fluoro-phenyl)-4-hydroxy-3-phenyl-1H-pyrrolo[2,3-c]pyridine-5-carbonyl]-amino}-acetic acid, {[7-methyl-1-(4-fluoro-phenyl)-4-hydroxy-3-phenyl-1H-pyrrolo[2,3-c]pyridine-5-carbonyl]-amino}-acetic acid, {[3-bromo-2-tert-butyl-1-(4-fluoro-phenyl)-4-hydroxy-1H-pyrrolo[2,3-c]pyridine-5-carbonyl]-amino}-acetic acid, {[2-tert-butyl-1-(4-fluoro-phenyl)-4-hydroxy-1H-pyrrolo[2,3-c]pyridine-5-carbonyl]-amino}-acetic acid, [(1-benzyl-4-hydroxy-2,3-dimethyl-1H-pyrrolo[2,3-c]pyridine-5-carbonyl)-amino]-acetic acid, [(2,3-dibromo-4-hydroxy-1-methyl-1H-pyrrolo[2,3-c]pyridine-5-carbonyl)-amino]-acetic acid, [(4-hydroxy-1,2,3-trimethyl-1H-pyrrolo[2,3-c]pyridine-5-carbonyl)-amino]-acetic acid, {[2-bromo-3-tert-butyl-1-(4-fluoro-phenyl)-4-hydroxy-1H-pyrrolo[2,3-c]pyridine-5-carbonyl]-amino}-acetic acid, {[3-tert butyl-1-(4-fluoro-phenyl)-4-hydroxy-1H-pyrrolo[2,3-c]pyridine-5-carbonyl]-amino}-acetic acid, [(1-benzyl-4-hydroxy-2,3-dipropyl-1H-pyrrolo[2,3-c]pyridine-5-carbonyl)-amino]-acetic acid, [(1-benzyl-3,7-dichloro-4-hydroxy-1H-pyrrolo[2,3-c]pyridine-5-carbonyl)-amino]-acetic acid, [(4-hydroxy-9-phenyl-9h-beta-carboline-3-carbonyl)-amino]-acetic acid, [(4-hydroxy-1-methyl-9-phenyl-9h-beta-carboline-3-carbonyl)-amino]-acetic acid, [(4-hydroxy-1,9-diphenyl-9h-beta-carboline-3-carbonyl)-amino]-acetic acid, [(1-benzyl-3-chloro-4-hydroxy-7-methyl-1H-pyrrolo[2,3-c]pyridine-5-carbonyl)-amino]-acetic acid, [(1-benzyl-3-chloro-4-hydroxy-7-phenyl-1H-pyrrolo[2,3-c]pyridine-5-carbonyl)-amino]-acetic acid, [(1-benzyl-3-chloro-7-ethyl-4-hydroxy-1H-pyrrolo[2,3-c]pyridine-5-carbonyl)-amino]-acetic acid, {[2-(4-fluoro-phenyl)-4-hydroxy-1,3-diphenyl-1H pyrrolo[2,3-c]pyridine-5-carbonyl]-amino}-acetic acid, [(3-chloro-4-hydroxy-1-phenyl-1H-pyrrolo[2,3-c]pyridine-5-carbonyl)-amino]-acetic acid, [(3-chloro-4-hydroxy-7-methyl-1-phenyl-1H-pyrrolo[2,3-c]pyridine-5-carbonyl)-amino]-acetic acid, {[1-(benzo[1,3]dioxol-5-ylmethyl)-3-bromo-2-(4-chloro-phenyl)-4-hydroxy-1H-pyrrolo[2,3-c]pyridine-5-carbonyl]-amino}-acetic acid, {[3-bromo-2-(4-chloro-phenyl)-4-hydroxy-1-phenyl-1H-pyrrolo[2,3-c]pyridine-5-carbonyl]-amino}-acetic acid, [(1-(benzo[1,3]dioxol-5-ylmethyl)-4-hydroxy-2-phenyl-1H-pyrrolo[2,3-c]pyridine-5-carbonyl)-amino]-acetic acid, {[1-(benzo[1,3]dioxol-5-ylmethyl)-2-(4-chloro-phenyl)-4-hydroxy-1H-pyrrolo[2,3-c]pyridine-5-carbonyl]-amino}-acetic acid, {[1-benzo[1,3]dioxol-5-ylmethyl-2-(4-chloro-phenyl)-4-hydroxy-3-methyl-1H-pyrrolo[2,3-c]pyridine-5-carbonyl]-amino)}-acetic acid, [(4-hydroxy-1,2-diphenyl-1H-pyrrolo[2,3-c]pyridine-5-carbonyl)-amino]-acetic acid, ([2-(4-chloro-phenyl)-4-hydroxy-3-methyl-1-phenyl-1H-pyrrolo[2,3-c]pyridine-5-carbonyl]-amino)-acetic acid, [(7-hydroxy-2-phenyl-thiazolo[4,5-c]pyridine-6-carbonyl)-amino]-acetic acid, [(7-hydroxy-2,4-diphenyl-thiazolo[4,5-c]pyridine-6-carbonyl)-amino]-acetic acid, [(7-hydroxy-4-methyl-2-phenyl-thiazolo[4,5-c]pyridine-6-carbonyl)-amino]-acetic acid, (S)-2-[(7-hydroxy-4-methyl-2-phenyl-thiazolo[4,5-c]pyridine-6-carbonyl)-amino]-propionic acid, {[7-hydroxy-2-(4-trifluoromethyl-phenyl)-thiazolo[4,5-c]pyridine-6-carbonyl]-amino}-acetic acid. {[2-(4-chloro-phenyl)-7-hydroxy-thiazolo[4,5-c]pyridine-6-carbonyl]-amino}-acetic acid, {[7-hydroxy-2-(4-methoxy-phenyl)-thiazolo[4,5-c]pyridine-6-carbonyl]-amino}-acetic acid, {[2-(4-fluoro-phenyl)-7-hydroxy-thiazolo[4,5-c]pyridine-6-carbonyl]-amino}-acetic acid, [(4-ethyl-7-hydroxy-2-phenyl-thiazolo[4,5-c]pyridin-6-carbonyl)-amino]-acetic acid, [(7-hydroxy-2-phenoxy-thiazolo[4,5-c]pyridine-6-carbonyl)-amino]-acetic acid, {[7-hydroxy-2-(methyl-phenyl-amino)-thiazolo[4,5-c]pyridine-6-carbonyl]-amino}-acetic acid, {[7-hydroxy-2-(phenylamino)-thiazolo[4,5-c]pyridine-6-carbonyl]-amino}-acetic acid, [(7-hydroxy-2-phenyl-thiazolo[5,4-c]pyridine-6-carbonyl)-amino]-acetic acid, {[2-(5-bromo-pyridin-3-yl)-7-hydroxy-thiazolo[4,5-c]pyridine-6-carbonyl]-amino}-acetic acid, [(7-hydroxy-2-pyridin-3-yl-thiazolo[4,5-c]pyridine-6-carbonyl)-amino]-acetic acid, [(4-butyl-7-hydroxy-2-phenyl-thiazolo[4,5-c]pyridine-6-carbonyl)-amino]-acetic acid, [(7-hydroxy-2-pyridin-2-yl-thiazolo[4,5-c]pyridine-6-carbonyl)-amino]-acetic acid, {[2-(4-fluoro-phenyl)-7-hydroxy-4-methyl-thiazolo[4,5-c]pyridin-6-carbonyl]-amino}-acetic acid, [(7-hydroxy-2-phenyl-4-propyl-thiazolo[4,5-c]pyridine-6-carbonyl)-amino]-acetic acid, {[7-hydroxy-2-(4-phenoxy-phenyl)-thiazolo[4,5-c]pyridine-6-carbonyl]-amino}-acetic acid, [(4-cyano-7-hydroxy-2-phenyl-thiazolo[4,5-c]pyridine-6-carbonyl)-amino]-acetic acid, [(7-hydroxy-4-isobutyl-2-phenyl-thiazolo[4,5-c]pyridine-6-carbonyl)-amino]-acetic acid, {[7-hydroxy-2-(3-methoxy-phenyl)-thiazolo[4,5-c]pyridine-6-carbonyl]-amino}-acetic acid, [(4-furan-2-yl-7-hydroxy-2-phenyl-thiazolo[4,5-c]pyridine-6-carbonyl)-amino]-acetic acid, [(7-hydroxy-2-phenyl-4-thiazol-2-yl-thiazolo[4,5-c]pyridine-6-carbonyl)-amino]-acetic acid, {[7-hydroxy-2-(2-methoxy-phenyl)-thiazolo[4,5-c]pyridine-6-carbonyl]-amino}-acetic acid, [(7-hydroxy-4-methyl-2-phenyl-thiazolo[5,4-c]pyridin-6-carbonyl)-amino]-acetic acid, {[2-(4-cyano-phenyl)-7-hydroxy-thiazolo[4,5-c]pyridine-6-carbonyl]-amino}-acetic acid, [(7-hydroxy-2,4-diphenyl-thiazolo[5,4-c]pyridine-6-carbonyl)-amino]-acetic acid, {[2-(3-chloro-4-fluoro-phenyl)-7-hydroxy-thiazolo[4,5-c]pyridine-6-carbonyl]-amino}-acetic acid, [(4-benzyl-7-hydroxy-2-phenyl-thiazolo[5,4-c]pyridine-6-carbonyl)-amino]-acetic acid, {[7-hydroxy-4-(4-morpholin-4 yl-phenyl)-2-phenyl-thiazolo[5,4-c]pyridine-6-carbonyl]-amino}-acetic acid, {[4-(4-cyano-phenyl)-7-hydroxy-2-phenyl-thiazolo[4,5-c]pyridine-6-carbonyl]-amino}-acetic acid, {[4-cyano-2-(4-fluoro-phenyl)-7-hydroxy thiazolo[4,5-c]pyridine-6-carbonyl]-amino}-acetic acid, {[4-cyano-7-hydroxy-2-(3-methoxy-phenyl)-thiazolo[4,5-c]pyridine-6-carbonyl]-amino}-acetic acid, [(4-cyano-7-hydroxy-2-phenyl-thiazolo[5,4-c]pyridine-6-carbonyl)-amino]-acetic acid, [(4-ethynyl-7-hydroxy-2-phenyl-thiazolo[4,5-c]pyridine-6-carbonyl)-amino]-acetic acid, [(4-acetyl-7-hydroxy-2-phenyl-thiazolo[4,5-c]pyridine-6-carbonyl)-amino]-acetic acid, [(7-hydroxy-2-phenyl-4-piperidin-1-yl-thiazolo[4,5-c]pyridine-6-carbonyl)-amino]-acetic acid, {[2-(4-tert-butyl-phenyl)-7-hydroxy-thiazolo[4,5-c]pyridine-6-carbony]-amino}-acetic acid, {[2-(2,3-dihydro-benzo[1,4]dioxin-6-yl)-7-hydroxy-thiazolo[4,5-c]pyridine-6-carbonyl]-amino}-acetic acid, [(2-benzo[b]thiophen-3-yl-7-hydroxy-thiazolo[4,5-c]pyridine-6-carbonyl)-amino]-acetic acid, [(2-biphenyl-4-yl-7-hydroxy-thiazolo[4,5-c]pyridine-6-carbonyl)-amino]-acetic acid, [(2-benzo[b]thiophen-2 yl-7-hydroxy-thiazolo[4,5-c]pyridine-6-carbonyl)-amino]-acetic acid, [(7-hydroxy-2-quinolin-3-yl-thiazolo[4,5-c]pyridine-6-carbonyl)-amino]-acetic acid, [(2-benzofuran-2-yl-7-hydroxy-thiazolo[4,5-c]pyridine-6-carbonyl)-amino]-acetic acid, [(2-dibenzofuran-4-yl-7-hydroxy-thiazolo[4,5-c]pyridine-6-carbonyl)-amino]-acetic acid, {[2-(2,3-dihydro-benzofuran-5-yl)-7-hydroxy-thiazolo[4,5-c]pyridine-6-carbonyl]-amino}-acetic acid, [(7-hydroxy-2-pyrimidin-5-yl-thiazolo[4,5-c]pyridine-6-carbonyl)-amino]-acetic acid, {[2-(1-benzyl-1H-pyrazol-4-yl)-7-hydroxy-thiazolo[4,5-c]pyridine-6-carbonyl]-amino}-acetic acid, {[2-(6-chloro-pyridin-3-yl)-7-hydroxy-thiazolo[4,5-c]pyridine-6-carbonyl]-amino}-acetic acid, {[2-(6-butoxy-pyridin-3-yl)-7-hydroxy-thiazolo[4,5-c]pyridine-6-carbonyl]-amino}-acetic acid, {[7-hydroxy-2-(6-phenylsulfanyl-pyridin-3-yl)-thiazolo[4,5-c]pyridine-6-carbonyl]-amino}-acetic acid, {[2-(1-benzyl-1H-pyrazol-4-yl)-4-cyano-7-hydroxy-thiazolo[4,5-c]pyridine-6-carbonyl]-amino}-acetic acid, {[2,3-dichloro-7-cyano-4-hydroxy-1-(3-methyl-butyl)-1H-pyrrolo[2,3-c]pyridine-5-carbonyl]-amino}-acetic acid, {[7-cyano-4-hydroxy-1-(3-methyl-butyl)-1H-pyrrolo[2,3-c]pyridine-5-carbonyl]-amino}-acetic acid, {[3-chloro-7-cyano-4-hydroxy-1-(3-methyl-butyl)-1H-pyrrolo[2,3-c]pyridine-5-carbonyl]-amino}-acetic acid, {[2,3-dichloro-7-cyano-1-cyclohexylmethyl-4-hydroxy-1H-pyrrolo[2,3-c]pyridine-5-carbonyl]-amino}-acetic acid, {[7-cyano-4-hydroxy-1-cyclohexylmethyl-1H-pyrrolo[2,3-c]pyridine-5-carbonyl]-amino}-acetic acid, [(1-benzyl-3-chloro-4-hydroxy-1H-pyrrolo[2,3-c]pyridine-5-cabonyl)-amino]-acetic acid, [(4-hydroxy-9-methyl-9H-beta-carboline-3-carbonyl)-amino]-acetic acid, [(4-hydroxy-1,9-dimethyl-9H-beta-carboline-3-carbonyl)-amino]-acetic acid, [(4-hydroxy-9-methyl-1-phenyl-9H-beta-carboline-3-carbonyl)-amino]-acetic acid, [(1-cyano-4-hydroxy-9-methyl-9H-beta-carboline-3-carbonyl)-amino]-acetic acid, {[3-bromo-7-cyan-2-(fluoro-phenyl)-4-hydroxy-1-phenyl-1H-pyrrolo[2,3-c]pyridine-5-carbonyl]-amino}-acetic acid, {[7-cyano-2-(4-fluoro-phenyl)-4-hydroxy-1-phenyl-1H-pyrrolo[2,3-c]pyridine-5-carbonyl]-amino}-acetic acid, [(4-hydroxy-5-phenyl-5H-pyrido[4,3-b]indole-3-carbonyl)-amino]-acetic acid, [(1-cyano-4-hydroxy-5-phenyl-5H-pyrido[4,3-b]indole-3-carbonyl)-amino]-acetic acid, [(4-hydroxy-1-methyl-5-phenyl-5H-pyrido[4,3-b]indole-3-carbonyl)-amino]-acetic acid, [(1-benzyl-3-chloro-7-cyano-4-hydroxy-1H-pyrrolo[2,3-c]pyridine-5-carbonyl)-amino]-acetic acid, {[3-cyano-2-(4-fluoro-phenyl)-4-hydroxy-1-phenyl-1H-pyrrolo[2,3-c]pyridine-5-carbonyl]-amino}-acetic acid, {[3-cyano-2-(4-fluoro-phenyl)-4-hydroxy-7-methyl-1-phenyl-1H-pyrrolo[2,3-c]pyridine-5-carbonyl]-amino}-acetic acid, {[3,7-dicyano-2-(4-fluoro-phenyl)-4-hydroxy-1-phenyl-1H-pyrrolo[2,3-c]pyridine-5-carbonyl]-amino}-acetic acid, [(7-cyano-4-hydroxy-1-phenyl-1H-pyrrolo[2,3-c]pyridine-5-carbonyl)-amino]-acetic acid, [(3-chloro-7-cyano-4-hydroxy-1-phenyl-1H-pyrrolo[2,3-c]pyridine-5-carbonyl)-amino]-acetic acid, {[2,3-dibromo-1-(4-fluoro-benzyl)-4-hydroxy-1H-pyrroto[2,3-c]pyridine-5-carbonyl]-amino}-acetic acid, [(4-hydroxy-1-phenethyl-1H-pyrrolo[2,3-c]pyridine-5-carbonyl)-amino]-acetic acid, {[2,3-dibromo-7-cyano-1-(4-fluoro-benzyl)-4-hydroxy-1H-pyrrolo[2,3-c]pyridine-5-carbonyl]-amino}-acetic acid, [(3-bromo-7-cyano-4-hydroxy-1-phenyl-1H-pyrrolo[2,3-c]pyridine-5-carbonyl)-amino]-acetic acid, {[7-cyano-1-(4-fluoro-benzyl)-4-hydroxy-1H-pyrrolo[2,3-c]pyridine-5-carbonyl]-amino}-acetic acid, [(3-chloro-7-cyano-4-hydroxy-1-phenethyl-1H-pyrrolo[2,3-c]pyridine-5-carbonyl)-amino]-acetic acid, {[2,3-dibromo-4-hydroxy-1-(1(S)-phenyl-ethyl)-1H-pyrrolo[2,3-c]pyridine-5-carbonyl]-amino}-acetic acid, {[3-chloro-7-cyano-1-(4-fluoro-benzyl)-4-hydroxy-1H-pyrrolo[2,3-c]pyridine-5-carbonyl]-amino}-acetic acid, [1-benzyl-2,3-dichloro-7-cyano-4-hydroxy-1H-pyrrolo[2,3-c]pyridine-5-carbonyl)-amino]-acetic acid, {[4-hydroxy-1-(1S-phenyl-ethyl)-1H-pyrrolo[2,3-c]pyridine-5-carbonyl]-amino}-acetic acid, [(2,3-dichloro-7-cyano-4-hydroxy-1-phenyl-1H-pyrrolo[2,3-c]pyridine-5-carbonyl)-amino]-acetic acid, [(2,3-dichloro-7-cyano-4-hydroxy-1-phenethyl-1H-pyrrolo[2,3-c]pyridine-5-carbonyl)-amino]-acetic acid, {[2,3-dichloro-7-cyano-4-hydroxy-1-(1S-phenyl-ethyl)-1H-pyrrolo[2,3-c]pyridine-5-carbonyl]-amino}-acetic acid, [1-benzyl-3-bromo-7-cyano-4-hydroxy-1H-pyrrolo[2,3-c]pyridine-5-carbonyl)-amino]-acetic acid, {[4-hydroxy-1-(1R-phenyl-ethyl)-1H-pyrrolo[2,3-c]pyridine-5-carbonyl]-amino}-acetic acid, {[4-hydroxy-1-(4-methoxy-benzyl)-1H-pyrrolo[2,3-c]pyridine-5-carbonyl]-amino}-acetic acid, {[7-cyano-4-hydroxy-1-(4-methoxy-benzyl)-1H-pyrrolo[2,3-c]pyridine-5-carbonyl]-amino}-acetyic acid, [(1-benzyl-7-cyano-4-hydroxy-3-methyl-1H-pyrrolo[2,3-c]pyridine-5-carbonyl)-amino]-acetic acid, {[2,3-dichloro-7-cyano-4-hydroxy-1-(4-methoxy-benzyl)-1H-pyrrolo[2,3-c]pyridine-5-carbonyl]-amino}-acetic acid, {[2,3-dichloro-7-cyano-4-hydroxy-1-(1R-phenyl-ethyl)-1H-pyrrolo[2,3-c]pyridine-5-carbonyl]-amino}-acetic acid, {[3-chloro-7-cyano-4-hydroxy-1-(4-methoxy-benzyl)-1H-pyrrolo[2,3-c]pyridine-5-carbonyl]-amino}-acetic acid, {[7-cyano-4-hydroxy-1-(4-methoxy-phenyl)-1H-pyrrolo[2,3-c]pyridine-5-carbonyl]-amino}-acetic acid, {[2,3-dichloro-7-cyano-4-hydroxy-1-(4-methoxy-phenyl)-1H-pyrrolo[2,3-c]pyridine-5-carbonyl]-amino}-acetic acid, {[3-chloro-7-cyano-4-hydroxy-1-(4-methoxy-phenyl)-1H-pyrrolo[2,3-c]pyridine-5-carbonyl]-amino}-acetic acid, {[(1-(4-fluoro-benzyl)-4-hydroxy-2,3-dimethyl-1H-pyrrolo[2,3-c]pyridine-5-carbonyl]-amino}-acetic acid, {[7-cyano-1-(4-fluoro-phenyl)-4-hydroxy-1H-pyrrolo[2,3-c]pyridine-5-carbonyl]-amino}-acetic acid, {[2,3-dichloro-7-cyano-4-hydroxy-1-(4-fluoro-phenyl)-1H-pyrrolo[2,3-c]pyridine-5-carbonyl]-amino}-acetic acid, {[3-chloro-7-cyano-4-hydroxy-1-(4-fluoro-phenyl)-1H-pyrrolo[2,3-c]pyridine-5-carbonyl]-amino}-acetic acid, {[1-(4-fluoro-benzyl)-4-hydroxy-1H-pyrrolo[2,3-c]pyridine-5-carbonyl]-amino}-acetic acid, [(2-cyano-4-hydroxy-1-phenyl-1H-pyrrolo[2,3-c]pyridine-5-carbonyl)-amino]-acetic acid, {[1-(2-fluoro-benzyl)-4-hydroxy-1H-pyrrolo[2,3-c]pyridine-5-carbonyl]-amino}-acetic acid, {[4-hydroxy-1-(2-methoxy-benzyl)-1H-pyrrolo[2,3-c]pyridine-5-carbonyl]-amino}-acetic acid, {[4-hydroxy-1-(3-methoxy-benzyl)-1H-pyrrolo[2,3-c]pyridine-5-carbonyl]-amino}-acetic acid, {[7-cyano-1-(4-fluoro-phenyl)-4-hydroxy-3-phenyl-1H-pyrrolo[2,3-c]pyridine-5-carbonyl]-amino}-acetic acid, {[7-cyano-1-(2-fluoro-benzyl)-4-hydroxy-1H-pyrrolo[2,3-c]pyridine-5-carbonyl]-amino}-acetic acid, {[7-cyano-1-(2-methoxy-benzyl)-4-hydroxy-1H-pyrrolo[2,3-c]pyridine-5-carbonyl]-amino}-acetic acid, {[7-cyano-1-(3-methoxy-benzyl)-4-hydroxy-1H-pyrrolo[2,3-c]pyridine-5-carbonyl]-amino}acetic acid, {[2-cyano-1-(3-fluoro-benzyl)-4-hydroxy-1H-pyrrolo[2,3-c]pyridine-5-carbonyl]-amino}-acetic acid, {[2,3-dichloro-7-cyano-1-(2-fluoro-benzyl)-4-hydroxy-1H-pyrrolo[2,3-c]pyridine-5-carbonyl]-amino}-acetic acid, {[1-(3-fluoro-benzyl)-4-hydroxy-1H-pyrrolo[2,3-c]pyridine-5-carbonyl]-amino}-acetic acid, {[3-chloro-7-cyano-1-(2-fluoro-benzyl)-4-hydroxy-1H-pyrrolo[2,3-c]pyridine-5-carbonyl]-amino}-acetic acid, {[3-chloro-7-cyano-4-hydroxy-1-(3-methoxy-benzyl)-1H-pyrrolo[2,3-c]pyridine-5-carbonyl]-amino}-acetic acid, {[7-cyano-1-(3-fluoro-benzyl)-4-hydroxy-1H-pyrrolo[2,3-c]pyridine-5-carbonyl]-amino}-acetic acid, {[7-cyano-1-(3,4-difluoro-benzyl)-4-hydroxy-1H-pyrrolo[2,3-c]pyridine-5-carbonyl]-amino}-acetic acid, {[3-chloro-7-cyano-1-(3,4-difluoro-benzyl)-4-hydroxy-1H-pyrrolo[2,3-c]pyridine-5-carbonyl]-amino}-acetic acid, {[2,3-dichloro-7-cyano-1-(3-fluoro-benzyl)-4-hydroxy-1H-pyrrolo[2,3-c]pyridine-5-carbonyl]-amino}-acetic acid, {[3-chloro-7-cyano-1-(3-fluoro-benzyl)-4-hydroxy-1H-pyrrolo[2,3-c]pyridine-5-carbonyl]-amino}-acetic acid, {[2,3-dichloro-7-cyano-1-(3,4-difluoro-benzyl)-4-hydroxy-1H-pyrrolo[2,3-c]pyridine-5-carbonyl]-amino}-acetic acid, [(1-benzyl-2,3-dichloro-7-hydroxy-1H-pyrrolo[3,2-c]pyridine-6-carbonyl)-amino]-acetic acid, [(2-tert-butyl-7-hydroxy-thiazolo[4,5-c]pyridine-6-carbonyl)-amino]-acetic acid, [(2-tert-butyl-7-hydroxy-4-methyl-thiazolo[4,5-c]pyridine-6-carbonyl)-amino]-acetic acid, [(2-tert-butyl-4-cyano-7-hydroxy-thiazolo[4,5-c]pyridine-6-carbonyl)-amino]-acetic acid, [(4-butyl-2-tert-butyl-7-hydroxy-thiazolo[4,5-c]pyridine-6-carbonyl)-amino]-acetic acid, [(2-tert-butyl-7-hydroxy-4-((E)-styryl)-thiazolo[4,5-c]pyridine-6-carbonyl)-amino]-acetic acid, [(2-tert-butyl-7-hydroxy-4-phenyl-thiazolo[4,5-c]pyridine-6-carbonyl)-amino]-acetic acid, [(2-tert-butyl-7-hydroxy-4-phenethyl-thiazolo[4,5-c]pyridine-6-carbonyl)-amino]-acetic acid, [(2-tert-butyl-7-hydroxy-4-isopropylsulfanylmethyl-thiazolo[4,5-c]pyridine-6-carbonyl)-amino]-acetic acid, [(7-hydroxy-2-methyl-4-phenyl-thiazolo[4,5-c]pyridine-6-carbonyl)-amino]-acetic acid, [(7-hydroxy-2-methyl-thiazolo[4,5-c]pyridine-6-carbonyl)-amino]-acetic acid, [(7-hydroxy-2-naphthalen-2-yl-thiazolo[4,5-c]pyridine-6-carbonyl)-amino]-acetic acid, [(7-hydroxy-2-thiophen-2-yl-thiazolo[4,5-c]pyridine-6-carbonyl)-amino]-acetic acid, and [(2-furan-2-yl-7-hydroxy thiiazolo[4,5-c]pyridine-6-carbonyl)-amino]-acetic acid.

Particular compounds suitable for use in the present inventions include [(4-Hydroxy-1-methyl-7-phenoxy-isoquinoline-3-carbonyl)-amino]-acetic acid (Compound A), [(1-Chloro-4-hydroxy-isoquinoline-3-carbonyl)-amino]-acetic acid (Compound B), {([5-(4-Chloro-phenoxy)-1-cyano-4-hydroxy-isoquinoline-3-carbonyl]-amino}-acetic acid (Compound C), {[4-Hydroxy-7-(4-methoxy-phenoxy)-isoquinoline-3-carbonyl]-amino}-acetic acid (Compound D), [(1-Cyano-4-hydroxy-5-phenoxy-isoquinoline-3-carbonyl)-amino]-acetic acid (Compound E), {[(2,3-Dichloro-7-cyano-4-hydroxy-1-(4-methoxy-benzyl)-1H-pyrrolo[2,3-c]pyridine-5-carbonyl]-amino}-acetic acid (Compound F), {[7-Cyano-1-(2-fluoro-benzyl)-4-hydroxy-1H-pyrrolo[2,3-c]pyridine-5-carbonyl]-amino}-acetic acid (Compound G), [(1-Cyano-4-hydroxy-7-isopropoxy-isoquinoline-3-carbonyl)-amino]-acetic acid (Compound H), {[1-Cyano-7-(2,6-dimethyl-phenoxy)-4-hydroxy-isoquinoline-3-carbonyl]-amino}-acetic acid (Compound I), [(7-Cyano-4-hydroxy-1-naphthalen-2-ylmethyl-1H-pyrrolo[2,3-c]pyridine-5-carbonyl)-amino]-acetic acid (Compound J), {[3-Bromo-7-cyano-4-hydroxy-1-(4-methoxy-benzyl)-1H-pyrrolo[2,3-c]pyridine-5-carbonyl]-amino}-acetic acid (Compound K), [(1-Chloro-4-hydroxy-7-trifluoromethyl-isoquinoline-3-carbonyl)-amino]-acetic acid, [(1-Chloro-4-hydroxy-5-methyl-isoquinoline-3-carbonyl)-amino]-acetic acid, [(7-Bromo-4-hydroxy-isoquinoline-3-carbonyl)-amino]-acetic acid, {[2-3,4′-Difluoro-biphenyl-4-ylmethyl)-5-hydroxy-6-isopropyl-3-oxo-2,3-dihydro-pyridazine-4-carbonyl]-amino}-acetic acid (Compound L), [(1-Hydroxy-4,4-dimethyl-3-oxo-3,4-dihydro-naphthalene-2-carbonyl)-amino]-acetic acid (Compound M), 4-Oxo-1,4-dihydro-[1,10]phenanthroline-3-carboxylic acid (Compound N), {[5-(3-Chloro-phenyl)-3-hydroxy-pyridine-2-carbonyl]-amino}-acetic acid (Compound O), and [(7-Fluoro-4-hydroxy-2-oxo-2H-thiochromene-3-carbonyl)-amino]-acetic acid (Compound P).

Suitable compounds for use in the methods and medicaments of the invention may be identified using any conventionally known methods. Suitable assay methods are well known in the art. For example, compounds may be tested for their ability to inhibit the activity of a HIF prolyl hydroxylase in an enzyme assay as described elsewhere herein. Compounds are combined with radiolabeled α-ketoglutarate, a hydroxylatable HIFα peptide, and a HIF prolyl hydroxylase, e.g., PHD2 under conditions where, in the absence of compound, the HIF prolyl hydroxylase is capable of hydroxylating the HIFα peptide and converting the α-ketoglutarate to succinate and carbon dioxide; and levels of liberated carbon dioxide are measured, wherein a reduction in the amount of liberated carbon dioxide in the presence of compound identifies an inhibitor of HIF prolyl hydroxylase. Methods of determining if any particular compound inhibits HIF prolyl hydroxylase are well known, for example, the methods described in U.S. Pat. No. 7,323,475.

### Methods for Identifyine Comnounds

A compound suitable for use in the method, or for manufacture of a medicament, of the invention is one that inhibits HIF hydroxylase activity. Methods for identifying compounds suitable for use in the method, or for manufacture of a medicament, of the invention are also provided. Assays for hydroxylase activity are standard in the art. Such assays can directly or indirectly measure hydroxylase activity. For example, an assay can measure hydroxylated residues, e.g., proline, etc., present in the enzyme substrate, e.g., a target protein, a synthetic peptide mimetic, or a fragment thereof. (See, e.g., Palmerini et al. (1985) J Chromatogr 339:285-292.) A reduction in hydroxylated residue, e.g., proline, in the presence of a compound is indicative of a compound that inhibits hydroxylase activity. Alternatively, assays can measure other products of the hydroxylation reaction, e.g., formation of succinate from 2-oxoglutarate. (See, e.g., Cunliffe et al (1986) Biochem J 240:617-619.) Kaule and Ounzler (1990; Anal Biochem 184:291-297) describe an exemplary procedure that measures production of succinate from 2-oxoglutarate.

Procedures such as those described above can be used to identify compounds that inhibit HIF hydroxylase activity. Target protein used in the assay may include HIFα or a fragment thereof e.g., HIF(556-575). Enzyme may include, e.g., HIF prolyl hydroxylase (see, e.g., GenBank Accession No. AAG33965, etc.) obtained from any source. Human HIF prolyl hydroxylase is preferred. Enzyme may also be present in a crude cell lysate or in a partially purified form. For example, procedures that measure HIF hydroxylase activity are described in Ivan et al. (2001, Science 292:464-468; and 2002, Proc Natl Acad Sci USA 99:13459-13464) and Hirsila et al. (2003, J Biol Chem 278:30772-30780); additional methods are described in International Publication No. WO 03/049686. Measuring and comparing enzyme activity in the absence and presence of the compound will identify compounds that inhibit hydroxylation of HIFα.

In certain aspects, a suitable compound is one that stabilizes HIFα. Compounds that inhibit HIF prolyl hydroxylase prevent or reduce the hydroxylation of one or more prolines of the HIFα subunit of the HIF protein. This lack of hydroxylated proline leads to the stabilization (often referred to as activation) of HIF. Determination of the stabilization of HIF by a compound can be used as an indirect measure of the ability of the compound to inhibit HIF prolyl hydroxylase. The ability of a compound to stabilize or activate HIFα can be measured, for example, by direct measurement of HIFα in a sample, indirect measurement of HIFα, e.g., by measuring a decrease in HIFα associated with the von Hippel Lindau protein (see, e.g., International Publication No. WO 2000/69908), or activation of HIF responsive target genes or reporter constructs (see, e.g., U.S. Pat. No. 5,942,434). Measuring and comparing levels of HIF and/or HIF-responsive target proteins in the absence and presence of the compound will identify compounds that stabilize HIFα and/or activate HIF. Suitable compounds for use in the present methods may be identified and characterized using the assay described in International Publication No. WO 2005/118836, or in Example 10 of International Publication No. WO 2003/049686, both of which are incorporated herein by reference in their entirety.

Compounds identifiable by these assays are specifically envisaged for use in the present invention.

### Pharmaceutical Formulations and Routes of Administration

The compositions and compounds suitable for use in the method, or for manufacture of a medicament, of the present invention can be delivered directly or in pharmaceutical compositions containing excipients, as is well known in the art.

A therapeutically effective amount, e.g., dose, of compound or drug can readily be determined by routine experimentation, as can an effective and convenient route of administration and an appropriate formulation. Various formulations and drug delivery systems are available in the art. (See, e.g., Gennaro, ed. (2000) Remington's Pharmaceutical Sciences, supra; and Hardman, Limbird, and Gilman, eds. (2001) The Pharmacological Basis of Therapeutics, supra.)

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

In preferred embodiments, for use in the method of the invention the compounds of the present invention are administered orally.

Pharmaceutical dosage forms of a suitable compound for use in the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., USP, JP, EP, and BP, FDA web page (www.fda.gov), Inactive Ingredient Guide 1996, and Handbook of Pharmaceutical Additives, ed. Ash; Synapse Information Resources, Inc. 2002.)

Pharmaceutical dosage forms of a compound for use in the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions for use in the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include, fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof (e.g., magnesium stearate), sugars (i.e. dextrose, sucrose, lactose, etc.), croscarmellose sodium, talc, tragacanth mucilage, vegetable oils (hydrogenated), microcrystalline cellulose, and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention can be administered topically, such as through a skin patch, a semi-solid or a liquid formulation, for example a gel, a (micro)-emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention are conveniently delivered in the form of a solution, suspension, emulsion, or semisolid aerosol from pressurized packs, or a nebuliser, usually with the use of a propellant, e.g., halogenated carbons derived from methane and ethane, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insuffiator, may be formulated. These typically contain a powder mix of the compound and a suitable powder base such as lactose or starch.

Compositions formulated for parenteral administration by injection are usually sterile and, can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Stable carriers for intravenous injection for the molecules of the invention are well-known in the art and include water-based solutions containing a base, such as, for example, sodium hydroxide, to form an ionized compound, sucrose or sodium chloride as a tonicity agent, for example, the buffer contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

For composition useful for the present methods of treatment, a therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

Dosages preferably fall within a range of circulating concentrations that includes the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects, i.e., minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

In some embodiments of the present invention, a therapeutically effective dose, or “effective amount,” for compounds for use in the invention include doses of 0.01 mg/kg, 0.1 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, or 50 mg/kg, and may include doses between these values, for example 1.5 mg/kg or 0.75 mg/kg. For administration in the methods of the present invention for reducing LDL-C, or reducing VLDL-C, etc., the doses may be adjusted during treatment to maintain a circulating LDL-C and/or VLDL-C level in the subject within a target range. Typical target ranges LDL-C and VLDL-C vary with the CHD risk level and other factors as described elsewhere herein and can be readily determined by competent medical practicioners.

In additional embodiments, effective treatment regimes for compounds of the invention include administration once a day; one, two or three times weekly; once a month; preferably once weekly. The dosing interval may be altered during the course of treatment, for example, the compound may be administered three times weekly initially for a number of weeks and then administered two times weekly, or once weekly.

The amount of agent or composition administered may be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack, or glass and rubber stoppers such as in vials. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### Example 1

### Effect of Compound a on Cholesterol Levels in CKD or ESRD Patients Treated for Anemia

Study 1 was Phase 2, randomized, open-label, dose titration, efficacy and safety study of compound A in non-dialysis chronic kidney disease (CKD) patients with anemia. The primary objectives of this study were to evaluate the efficacy and safety of the compound in the correction of anemia (i.e. increasing hemoglobin levels) in non-dialysis CKD patients. The study included six dosing cohorts of approximately 24 subjects each. Subjects in Study 1 received compound A in doses ranging from 1.0 mg/kg to 2.5 mg/kg, in frequencies of once, twice, or three times weekly. Cohort A and cohort B both received a weight-adjusted dose of approximately 1 mg/kg, three times a week for 16 weeks; the dosing in cohort B was reduced to twice a week once anemia was corrected. Cohort C and cohort D received a fixed dose of 50 mg or 100 mg, respectively, three times a week, for 24 weeks. Cohort E received a weight-adjusted dose of approximately 1 mg/kg twice a week, reduced to once a week once anemia was corrected. Cohort F received a fixed dose of 70 mg, three times a week. Administration of compound A in all cohorts effectively corrected hemoglobin levels in CKD patients.

Study 2 was a phase 2, randomized, open-label active-comparator (Epoetin alfa (EPO)) and double-blind placebo-controlled, dose-ranging safety and exploratory efficacy study of compound A in subjects with end-stage renal disease (ESRD) receiving maintenance hemodialysis. The primary objective was to evaluate the efficacy and safety of the compound in maintaining and/or correcting hemoglobin (Hb) in subjects with ESRD on maintenance hemodialysis therapy, previously treated with intravenous EPO. Subjects on Study 2 received compound A in doses ranging from 0.8 mg/kg to 3.0 mg/kg three times a week. Administration of compound A effectively corrected hemoglobin levels in ESRD patients on dialysis.

In analyzing the results of the Study 1 and Study 2 trials with respect to the efficacy for treatment of anemia, the present inventors unexpectedly observed reductions in the total circulating cholesterol levels of subjects receiving compound A for treatment of anemia. Follow-up analysis showed significant reductions in LDL-C, VLDL-C and triglyceride levels. HDL-C levels were unchanged or slightly reduced. Total cholesterol and lipid panels were measured by standard procedures.

### Total Cholesterol—All Subjects

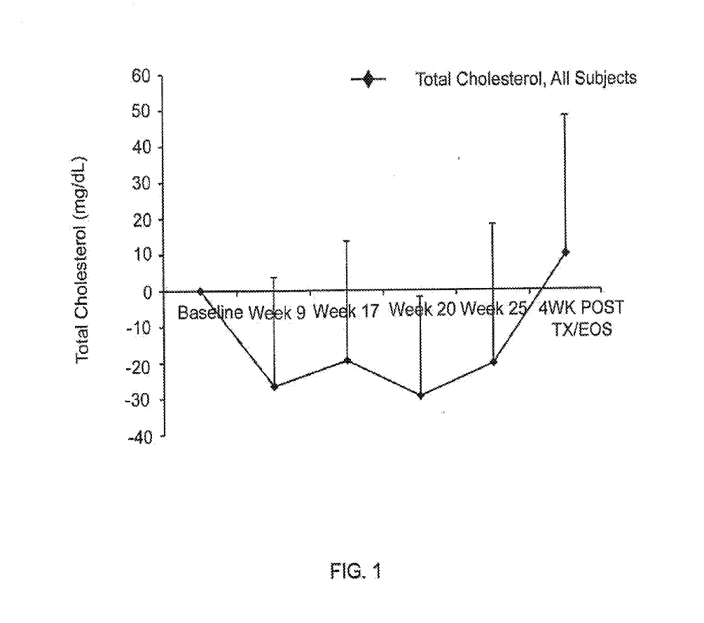

Total cholesterol was measured from all subjects in Study 1 or Study 2 as part of the chemistry panel at prespecified time points. FIG. 1 shows the total cholesterol over time for all subjects from all cohorts of Study 1 (n=144 at baseline). There was a significant decrease in total cholesterol initially during study treatment, which returns back to baseline after treatment ended (last time point is 4 weeks after the last dose of compound A was given). Total cholesterol decreased initially with treatment, reached a plateau where it remained during treatment, and then returned to baseline after treatment ends. The reduction in total cholesterol levels was compound A dose dependent (data not shown).

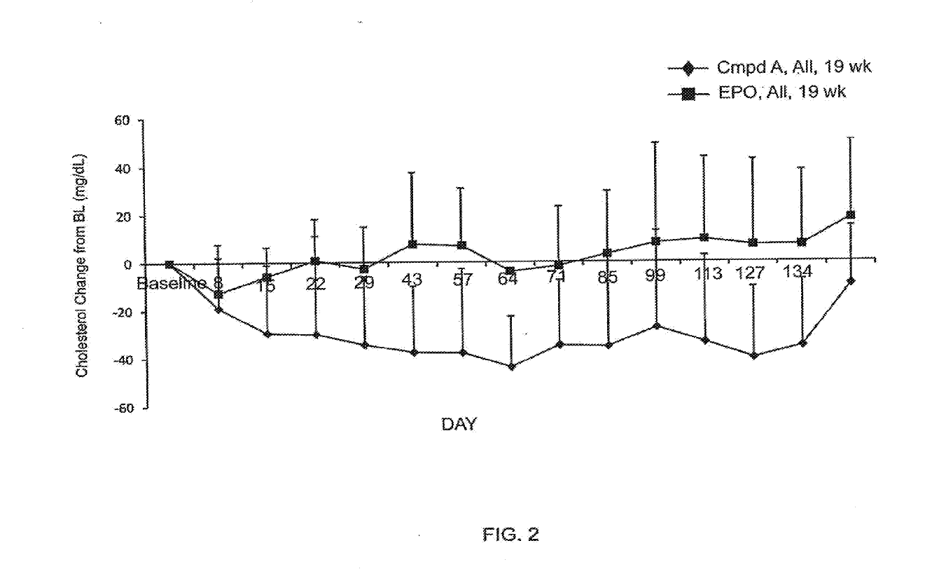

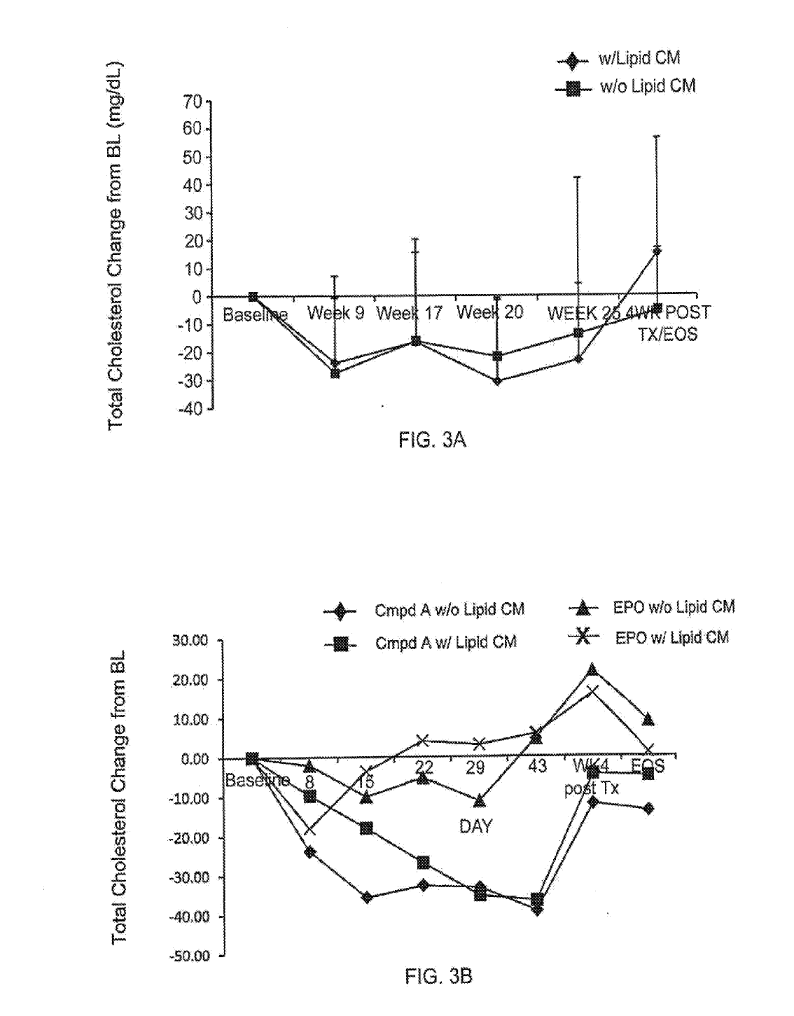

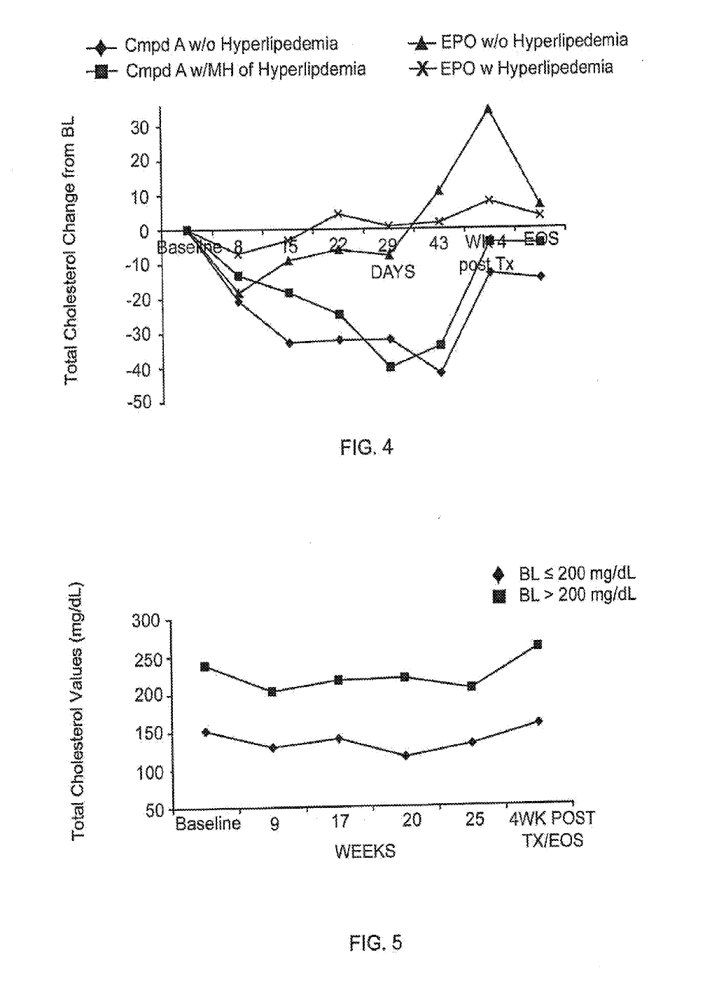

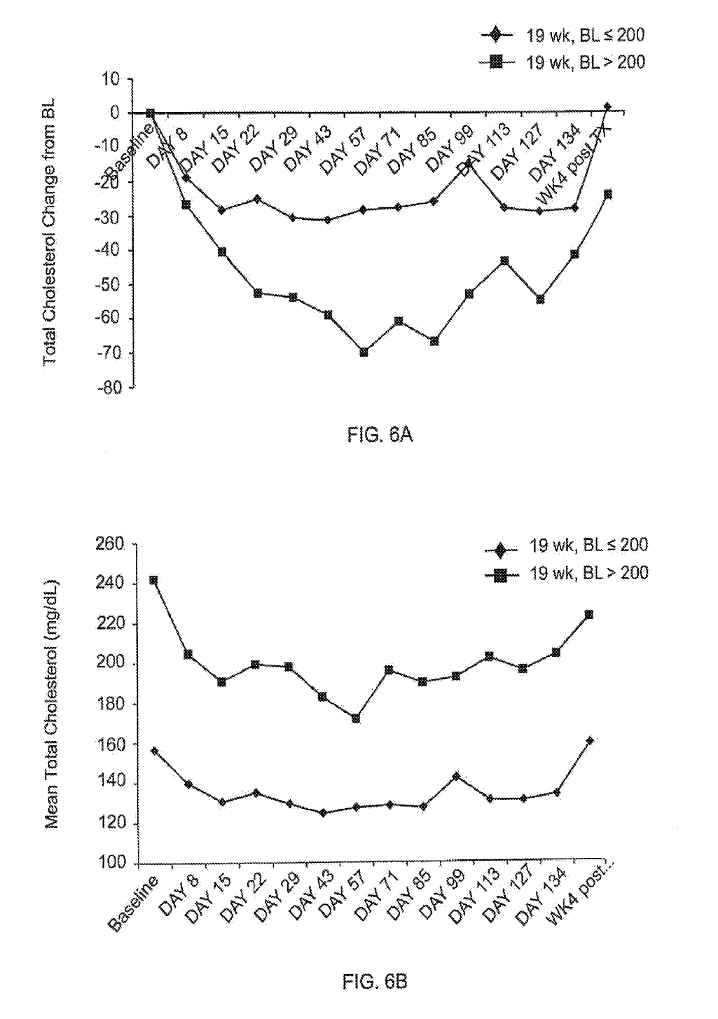

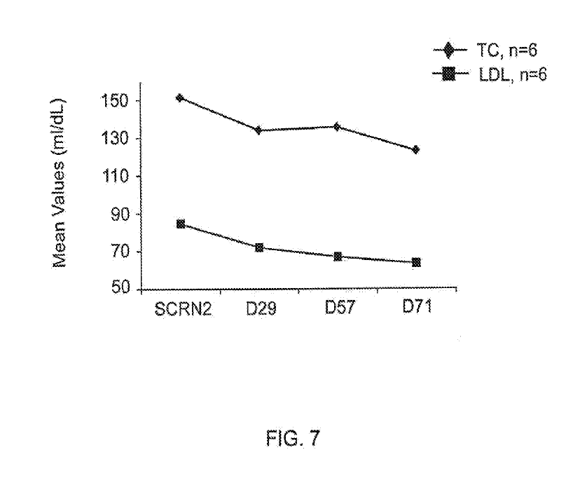

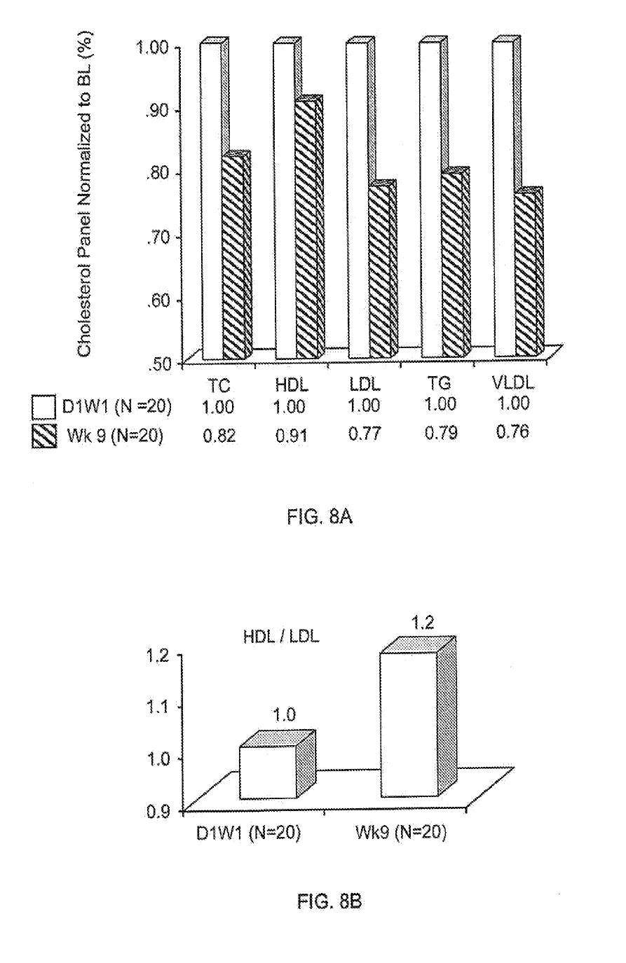

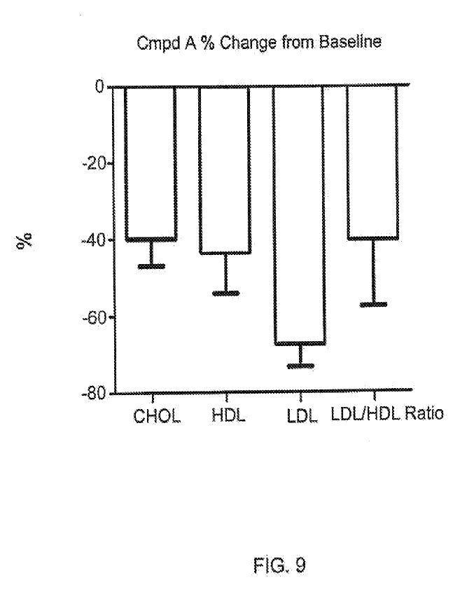

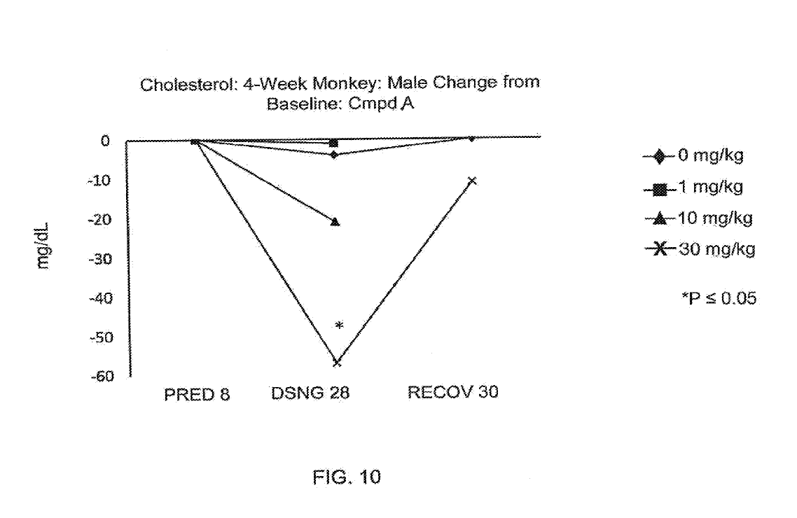

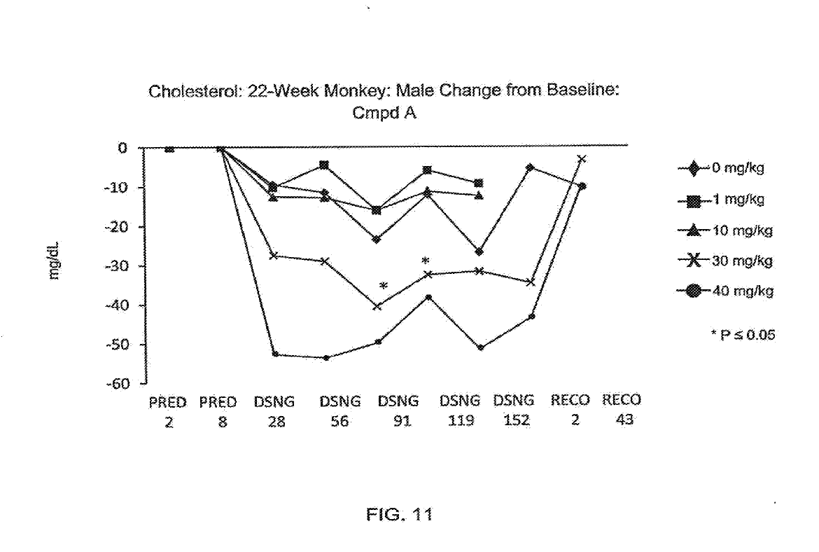

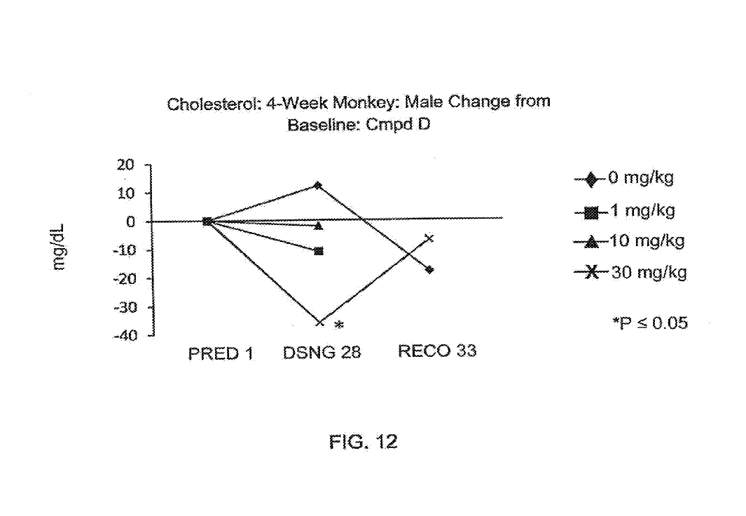

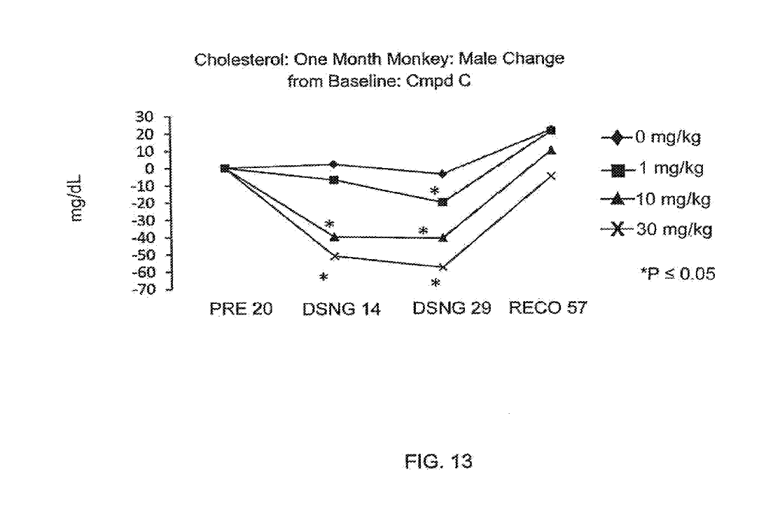

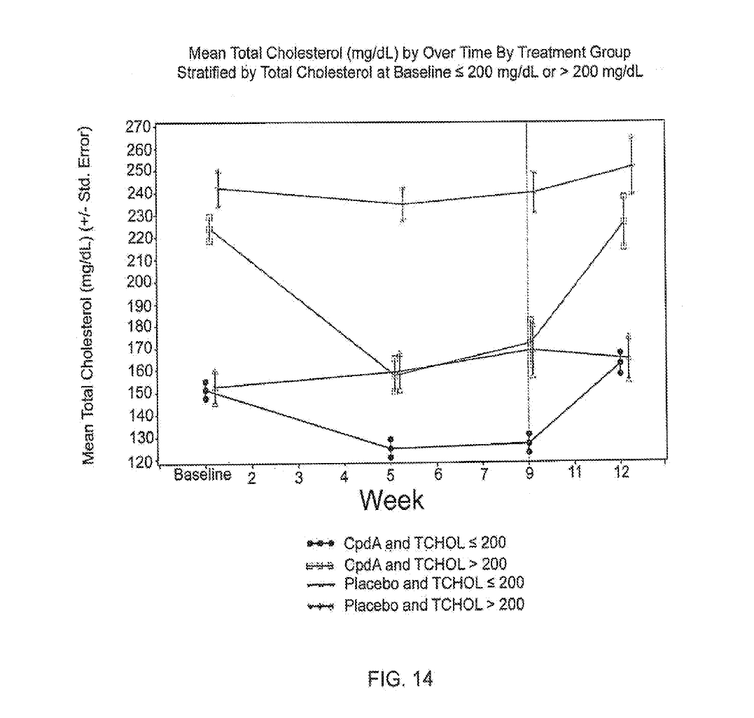

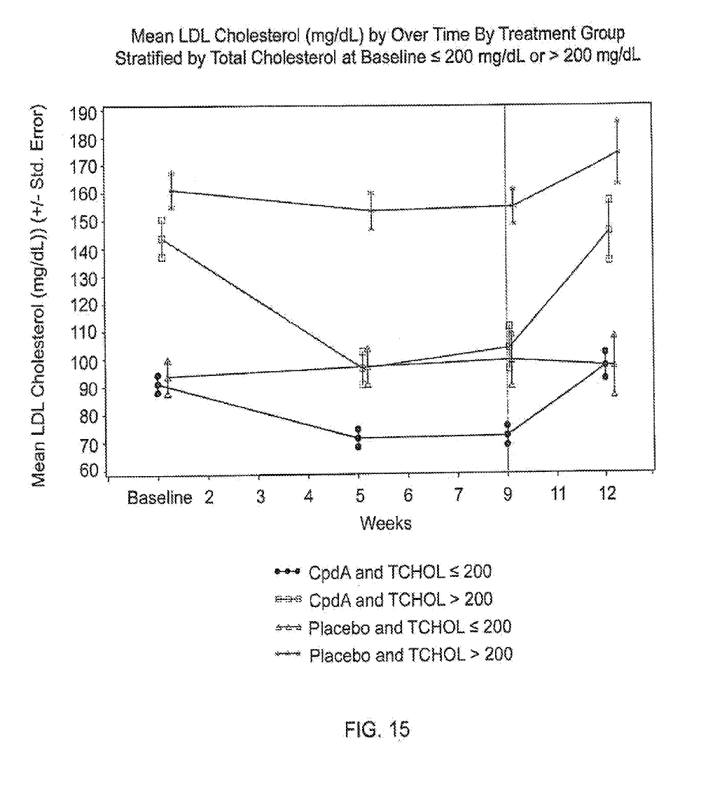

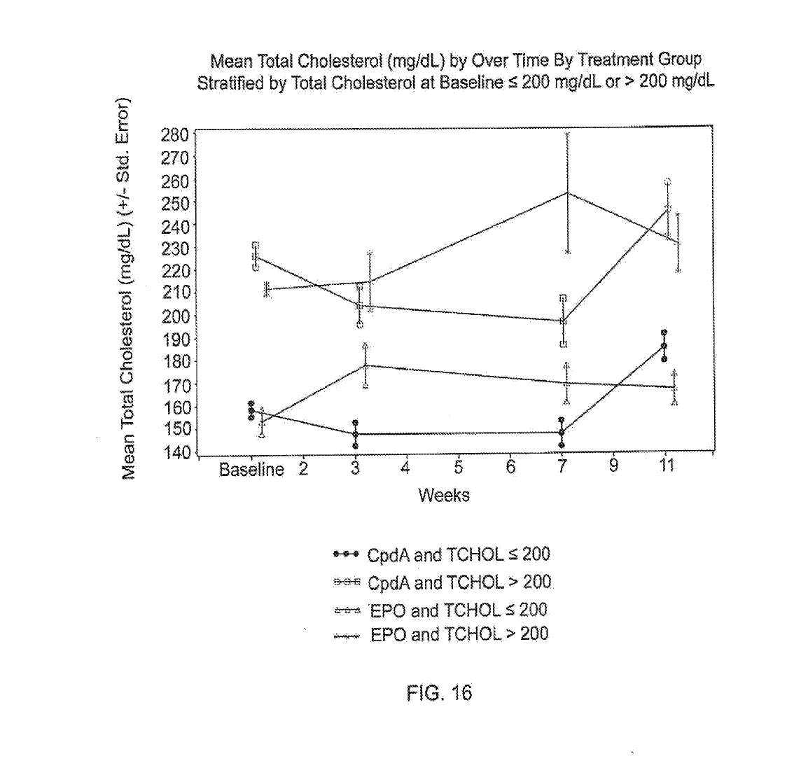

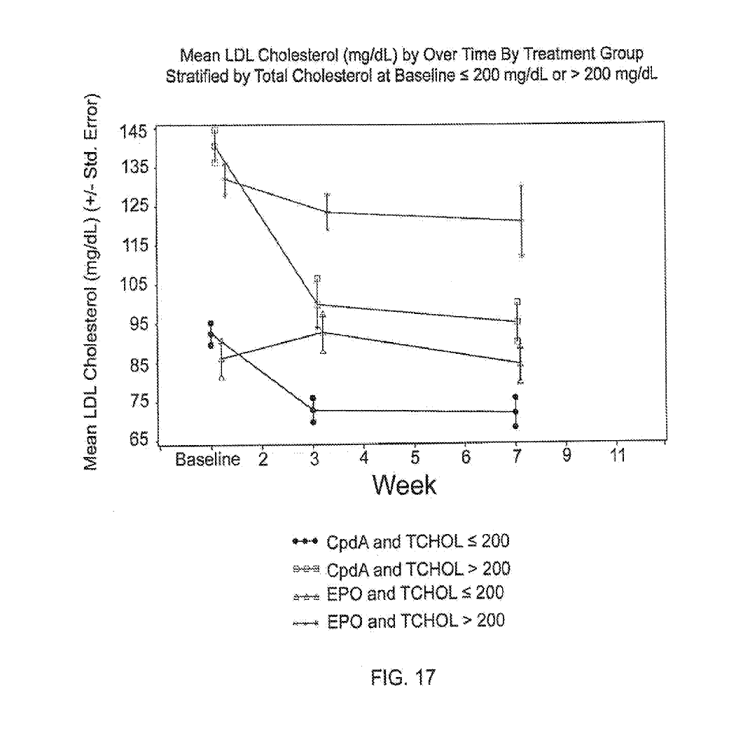

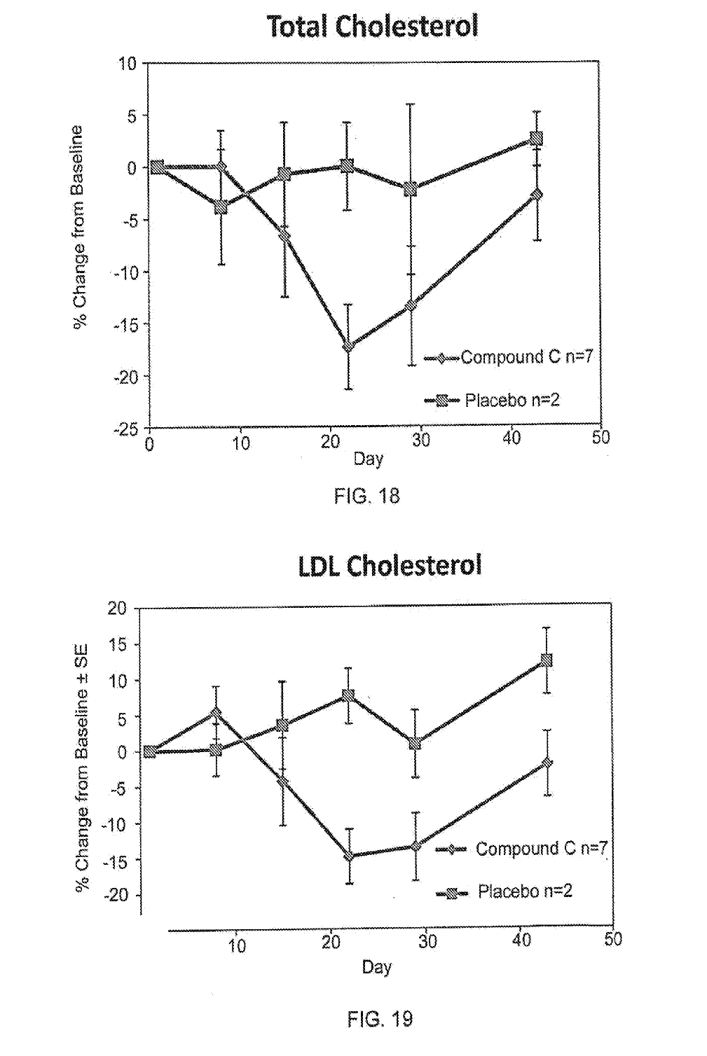

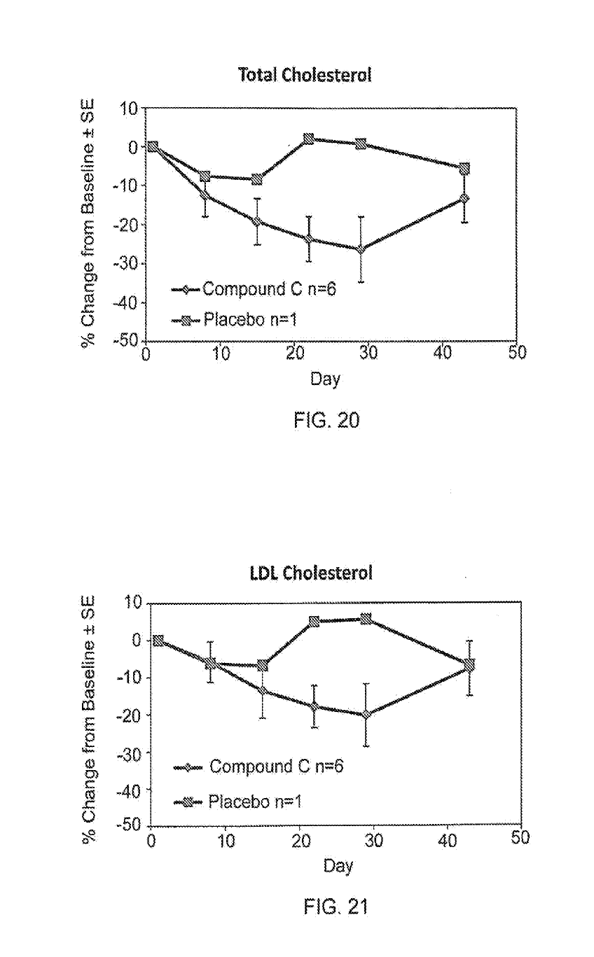

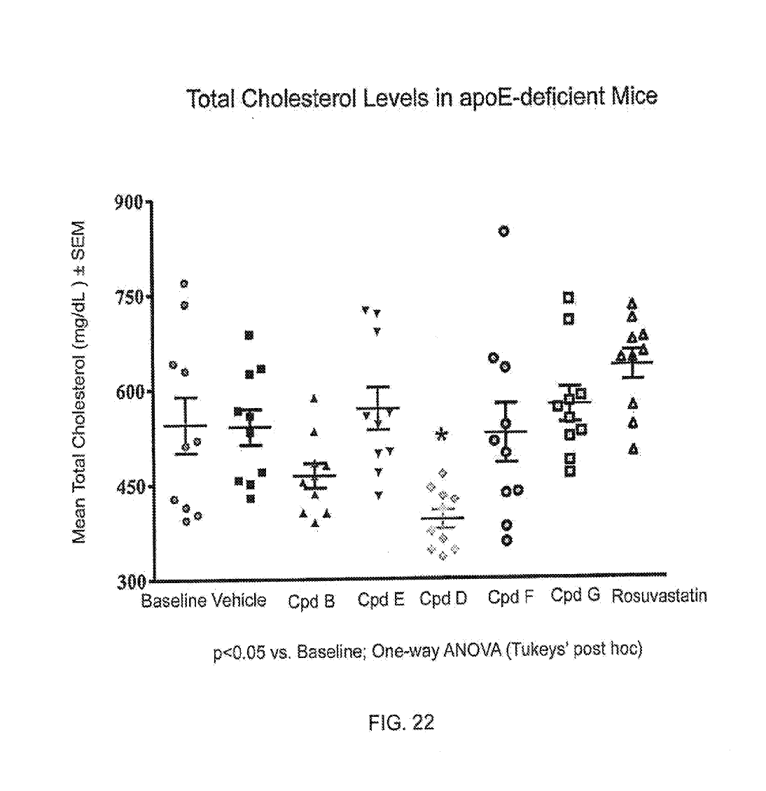

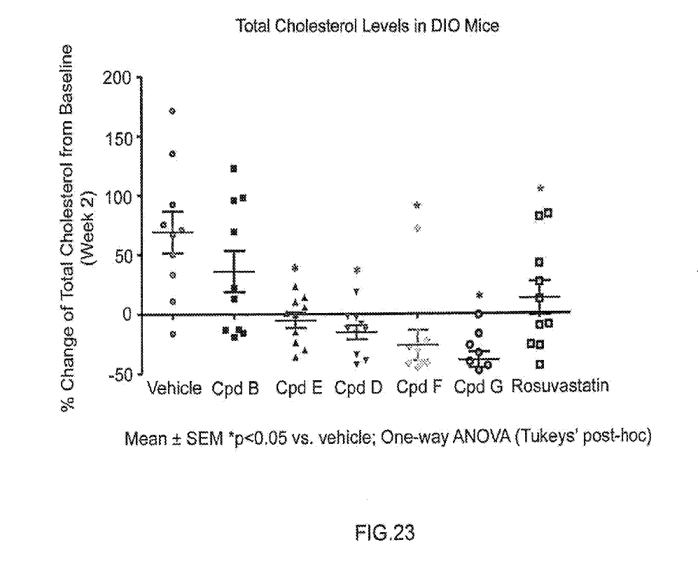

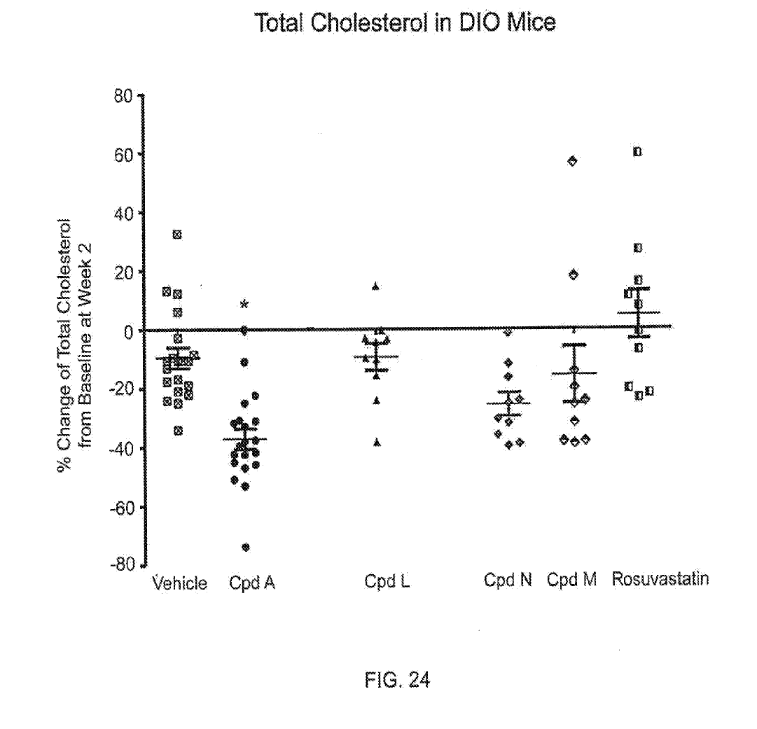

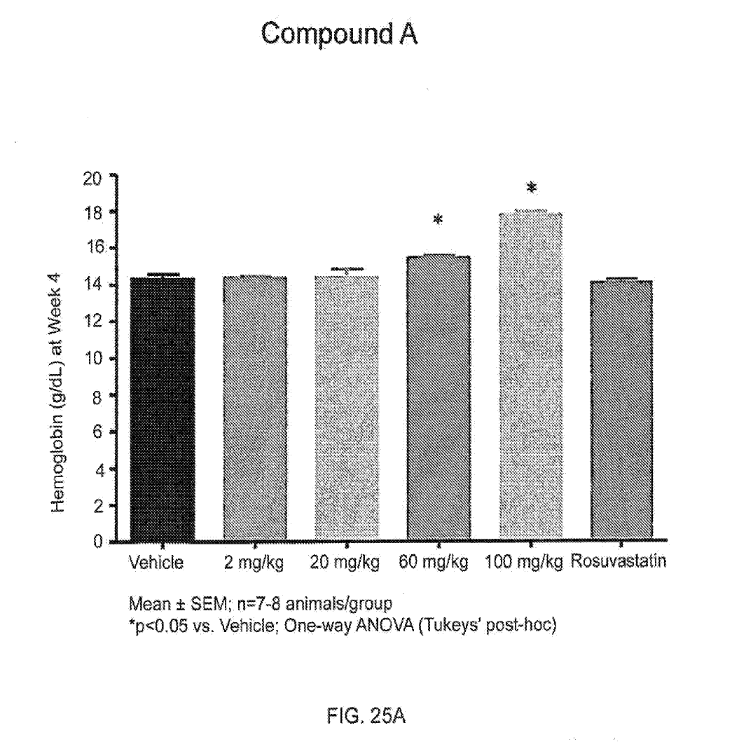

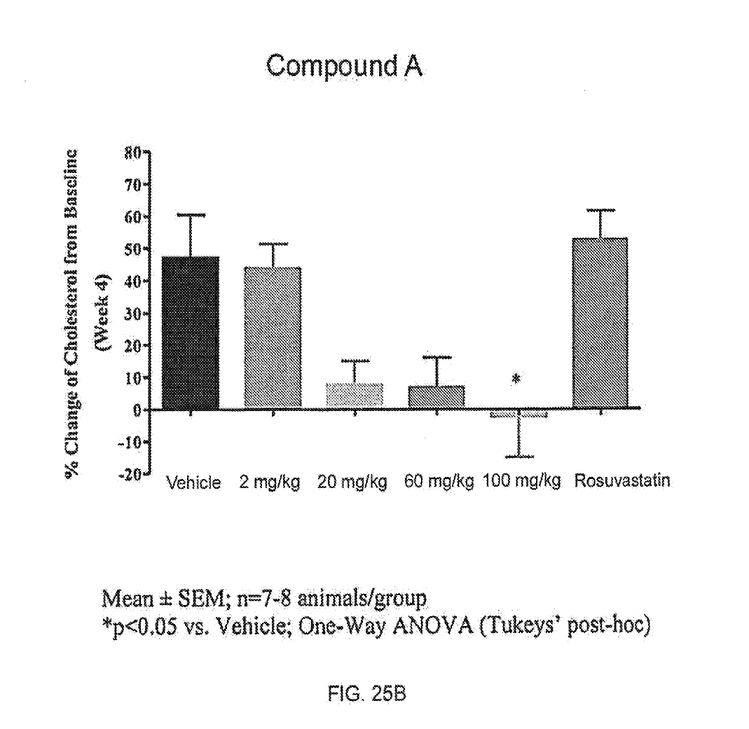

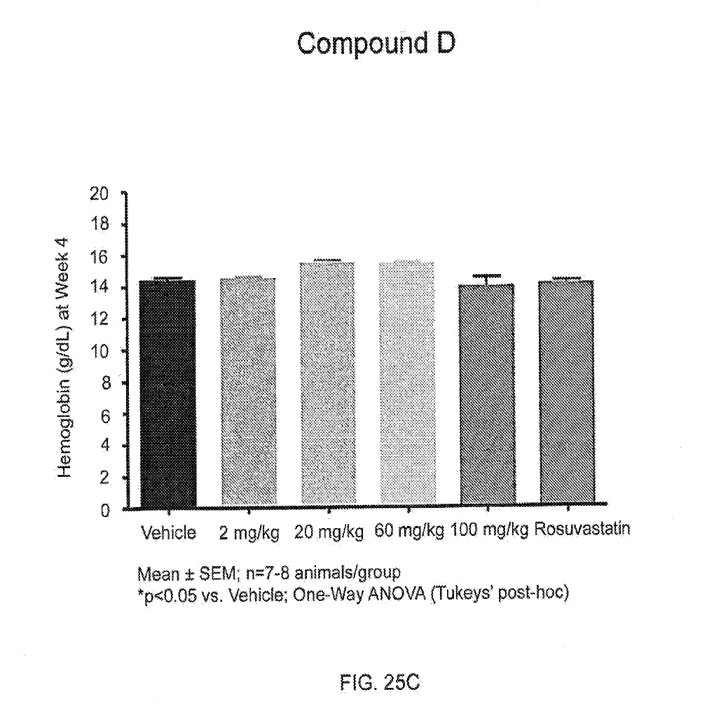

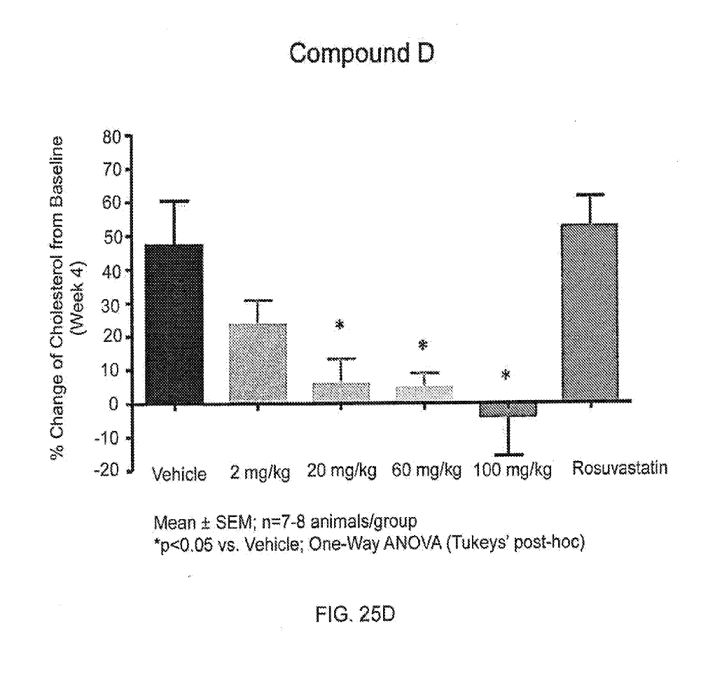

FIG. 2 shows the change in total cholesterol from baseline over time for subjects from Study 2 treated with compound A (n=58) or EPO (n=14). There was a significant decrease in total cholesterol in subjects treated with compound A for 19 weeks. Total cholesterol returned to baseline after treatment ended. No reduction of total cholesterol was observed in subjects treated with EPO for the same time period. Similar results were observed for subjects who received 6 week treatment.

### Subjects on Cholesterol-Lowering Medications

In both Study 1 and Study 2, there was a significant number of subjects who were taking cholesterol-lowering medications such as statins or fibrates. The effect on total cholesterol of treatment with compound A in subjects also taking cholesterol-lowering medications and in subjects not taking cholesterol-lowering medications was analyzed.

For Study 1, n=102 at baseline for subjects with cholesterol-lowering medication, n=42 for subjects not on cholesterol-lowering medication. For Study 2, n=50 (Compound A) or n=19 (EPO) at baseline for subjects with cholesterol-lowering medication, n=51 (Compound A) or n=14 (EPO) for subjects not on cholesterol-lowering medication.

The results of the analysis are shown in FIGS. 3A (for Study 1) and 3B (for Study 2). These results demonstrate that the effect of compound A on cholesterol levels is not significantly different for subjects who are already taking other cholesterol-lowering medications from subjects who are not taking such medications. Both groups showed a similar reduction in total cholesterol upon treatment with compound A. The reduction in total cholesterol is a result of reduction in the LDL-C, VDL-C, and triglyceride levels. The HDL-C levels are unchanged or only slightly reduced. In Study 1, for 4 patients who were already taking a cholesterol-lowering medication (statin) but whose LDL-C levels were still undesirably above 100 mg/dL, administration of compound A was associated with further LDL-C reductions ranging from 7.0% to 33%.

Subjects with Prior Medical History of Hyperlipidemia and/or Hypercholesteremia

The results from Study 2 (6 weeks) were stratified for subjects with a prior medical history of hyperlipidemia and/or hypercholesterolemia (n=46 for Compound A; n=19 for EPO), or not (n=55 for Compound A; n=14 for EPO). The results for that analysis are shown in FIG. 4. Treatment with compound A reduced the total cholesterol level of subjects in similar fashion whether or not the subjects had a prior medical history of hyperlipidemia and/or hypercholesterolemia.

Subjects with BL Cholesterol ≦200 mg/dL or >200 ma/dL

The results from both Study I and Study 2 were further analyzed for subjects having a baseline total cholesterol level of ≦200 mg/dL (n=103 for Study 1; n—=51 (compound A), n=17 (EPO) for Study 2) and those having a baseline total cholesterol of >200 mg/dL (n=31 for Study 1; n=13 (compound A), n=3 (EPO) for Study 2). FIG. 5 shows that total cholesterol is reduced in each group of CKD patients (Study 1) regardless of the cholesterol level at baseline. FIGS. 6 A and B show the change from baseline and the total cholesterol in Study 2 (ESRD) subjects. The reduction in total cholesterol appears to be larger for the subjects with higher cholesterol at BL. In all subjects the total cholesterol returned to baseline after treatment was ended. Data shown is for 19-week study. Similar results were observed for subjects who received 6-week treatment.

### B. Lipid Panels

Samples collected for various biomarker analyses per protocol in each study were stored frozen. Selected subsets of samples from the stored frozen samples were assayed for lipid profiling.

Frozen serum samples, collected from subjects in Study 1 or Study 2, were selected for the lipid panel analysis based on the following criteria: 1) with matched baseline (D1W1) and; 2) at least one other time point. The complete lipid panel includes: total cholesterol (TC), HDL, LDL-measured, Triglycerides and VLDL (Calculated). Ratio of HDL/LDL or LDL/HDL were analyzed.

### Study 1 (CKD Patients)

A total of n=9 subjects samples were selected and assayed for the complete lipid panel as described above. Subjects on this study protocol were not required to fast nor have any diet restrictions.

Results from six subjects with identical time points are shown in FIG. 7. Both total cholesterol and LDL were shown to reduce over the course of treatment.

### Study 2 (ESRD Patients)

A total of n=20 subjects were selected and assayed for the complete lipid panel as described above. Subjects on this study protocol were not required to fast nor have any diet restrictions.

There is significant reduction of total cholesterol, LDL, triglycerides and VLDL, with slight decrease of HDL. FIG. 8A shows each component of the lipid panel normalized to BL. FIG. 8B demonstrates a 20% increase in HDL/LDL ratio.

Subgroup analysis of subjects whose BL total cholesterol either fall into 1) ≦200 mg/dL or 2) >200 mg/dL were also performed. The reductions in the lipid profiles were comparable between the two groups. The data in Table 1 demonstrates that there is a significant reduction of total cholesterol and LDL-C level at week 9 comparing to Day1 Week1 (Baseline).

TABLE 1

| | | | Change | | | Change | |
|---|---|---|---|---|---|---|---|
| | | | from | Ratio | | from | Ratio |
| | Visit | TC | BL | of BL | LDL | BL | of BL |
| BL | D1W1 | 157.90 | 0.00 | 1.00 | 81.20 | 0.00 | 1.00 |
| ≦200 | W9 | 128.20 | −29.70 | 0.81 | 62.20 | −19.00 | 0.77 |
| BL >200 | D1W1 | 275.80 | 0.00 | 1.00 | 203.40 | 0.00 | 1.00 |
| | W9 | 239.20 | −36.60 | 0.87 | 164.20 | −39.20 | 0.81 |

### Example 2

### Reduction in Circulating LDL-C in Healthy Subjects

Plasma (sodium heparin anticoagulant) samples were taken from subjects dosed orally twice per week with placebo (n=6), 0.75 mg/Kg (n=6), or 1.88 mg/Kg (n=6) of Compound A. Compound A was administered on Day I after an overnight fast. Subjects were fasted overnight prior on subsequent dosing days. Overnight fasting was not required on non-dosing days. The samples had been stored frozen at −70° C. for up to 6 years, and had undergone up to two thaw/freeze cycles prior to testing. Results are presented separately for samples collected during fasting: Day 1 (0, 1, 2 hrs post-dose), Day 2 (24 hrs), Day 3 (72 hrs), Day 7 (168 hrs), Day 10 (240 hrs) and Day 17 (408 hours).

Samples were analyzed using validated assays on a Roche Modular system. Total cholesterol was measured using the CHOD-PAP reagent from Roche, Cat. No. 11875540216. HDL cholesterol was measured using reagents from Polymedco, Cat. No. 9400 (data not shown). LDL cholesterol was measured using the LDL-C plus 2^{nd} generation reagents from Roche, Cat. No. 04711220190. Triglycerides were measured using GPO-PAP reagent from Roche, Cat. No. 11730711216 (data not shown).

The average values for total plasma cholesterol and LDL cholesterol (in mg/dL) are presented in Table 2. Standard deviations of the average values are shown in the parentheses. These data show that both total cholesterol and LDL-C was reduced by treatment with compound A.

TABLE 2

| | Time, | Total Cholesterol mg/dL | | | LDL Cholesterol mg/dL | | |
|---|---|---|---|---|---|---|---|
| | hours | 0 mg/Kg | 0.75 mg/Kg | 1.88 mg/Kg | 0 mg/Kg | 0.75 mg/Kg | 1.88 mg/Kg |
| Compound A | 0 | 167 (27) | 159 (19) | 165 (33) | 92 (24) | 89 (16) | 95 (36) |
| | 1 | 170 (29) | 161 (20) | 164 (30) | 98 (22) | 90 (18) | 95 (36) |
| | 2 | 177 (28) | 155 (20) | 167 (32) | 103 (22) | 87 (15) | 97 (35) |
| | 24 | 174 (27) | 159 (17) | 161 (31) | 98 (23) | 91 (19) | 96 (36) |
| | 72 | 167 (29) | 145 (12) | 154 (26) | 93 (27) | 80 (10) | 92 (27) |
| | 168 | 173 (22) | 145 (11) | 144 (24) | 96 (19) | 81 (13) | 77 (31) |
| | 240 | 165 (27) | 149 (16) | 144 (23) | 97 (24) | 86 (7) | 78 (29) |
| | 408 | 176 (28) | 136 (32) | 152 (22) | 100 (26) | 70 (17) | 86 (29) |

### Example 3

### Effect of Single Dose Compound A on Lipid Panel in Rats

The effects of a single oral dose of compound A on changes from baseline levels of cholesterol, HDL, LDL and LDL/HDL ratio were evaluated in Sprague-Dawley rats. The rats (6 rats/dose group) were administered a 60 mg/kg dose of Compound A and then food fasted overnight. Blood samples were collected at baseline (pre-dose) and approximately 24 hours following dosing to determine levels of cholesterol, HDL, LDL and the LDL/HDL ratio.

Data generated in this study show that a single oral dose of 60 mg/kg Compound A administered to fasted Sprague Dawley rats results in a significant decrease from baseline at 24 hrs after single dose administration for total cholesterol, LDL and the LDL/HDL ratio. After 24 hours, total cholesterol decreased 26±9 mg/dL, HDL decreased 21±7 mg/dL, LDL decreased 11±3 mg/dL and the LDL/HDL ratio decreased 0.14±0.08. The mean percent decrease ±SD from baseline for each parameter evaluated is illustrated in FIG. 9.

### Example 4

### Effect on Cholesterol Levels after Repeated Administration of PHI in Monkeys

The effects of Compound A, Compound D and Compound C on blood cholesterol levels in monkeys were evaluated following repeated oral administration of each compound to cynomolgus monkeys in a number of toxicity studies. The durations of the studies were 28-days with daily dosing (Compound A, Compound D and Compound C) and 22-weeks (Compound A) with intermittent dosing every three days (Monday, Wednesday, and Friday). During these studies monkeys were fasted prior to each blood collection. Plasma samples were then evaluated at pre-dose and at different time-points for levels of total cholesterol.

The results of these studies demonstrate that total cholesterol was decreased in a dose-dependent manner following repeated administration of each of the compounds tested. The data are presented as a mean decrease ±SD from baseline in FIGS. 10-13. Statistically significant changes from pre-dose are noted as * in the Figures. Only data from male animals are shown, however similar decreases were observed for females in each of the studies. Also, decreases in cholesterol were observed and maintained for up to one year in an additional monkey study with Compound A.

The dose groups evaluated in each of the studies were:
1) Compound A 28-Day study: 0, 1, 10 and 30 mg/kg; 5 monkeys/sex/group (FIG. 10). This study shows dose-dependent decrease in cholesterol levels at day 28 after daily dosing. Animals were recovered for 30 days and cholesterol levels returned back to baseline (pre-dose) indicating that this effect was related to compound A administration.
2) Compound A 22-week study: 0, 1, 10, 30, and 40 mg/kg; 5 monkeys/sex/group (FIG. 11). This study shows dose-dependent decrease in cholesterol levels starting at day 28 to day 152 after intermittent dosing. Animals were recovered for 43 days and cholesterol levels returned back to baseline (pre-dose) indicating that this effect was related to compound A administration.
3) Compound D 28-Day study: 0, 1, 10, and 30 mg/kg; 5 monkeys/sex/group for 0 and 30 mg/kg; 3 monkeys/sex/group for 1 and 10 mg/kg) (FIG. 12). This study shows dose-dependent decrease in cholesterol levels at day 28 after daily dosing. Animals were recovered for 33 days and cholesterol levels returned back to baseline (pre-dose) indicating that this effect was related to compound D administration.
4) Compound C 28-Day study: 0, 1, 10, and 30 mg/kg; 5 monkeys/sex/group (FIG. 13). This study shows dose-dependent decrease in cholesterol levels at day 14 and 29 after daily dosing. Animals were recovered for 57 days and cholesterol levels returned back to baseline (pre-dose) indicating that this effect was related to compound C administration.

### Example 5

### Effect of Compound C and Compound G on Lipid Panel in Monkeys

The test compounds (C and G) were administered at a dose of 30 mg/kg to cynomolgus monkeys (3 monkeys/dose group) via intravenous (IV) injection or oral gavage (PO). The same monkeys were administered the test compounds in sequence with a washout period between administration of each compound. Monkeys were fasted overnight prior to dosing and food was withheld during the first 4 hours after dosing and food was then allowed. Blood samples were collected at baseline prior to dosing, 4, 12, 24, 48 and 72 hrs after HIF-PHI administration. Table 3 shows change from baseline (±SD) at 24 hrs post-dose. There were a total of 6 monkeys in each dose group, 3 that were dosed PO and 3 dosed IV.

Cholesterol levels were decreased at 24 hours following administration of each compound. Differences in regulation of HDL, LDL and the LDL/HDL ratio for the compounds tested are apparent. These data show that the compounds reduced the circulating level of LDL-C, and decreased the LDL/HDL ratio.

TABLE 3

| Lipid Panel | C | G |
|---|---|---|
| Cholesterol (mg/dL) | −11.8 ± 4.9 | −6.5 ± 10.4 |
| HDL (mg/dL) | 0.5 ± 6.9 | 0.17 ± 5.4 |
| LDL (mg/dL) | −12.5 ± 6.3 | −8.7 ± 5.9 |
| LDL/HDL ratio | −0.17 ± 0.12 | −0.13 ± 0.08 |
| TGs (mg/dL) | −10 ± 21 | −23 ± 8 |

### Example 6

### Effect of PHI Compounds on Expression of Genes Involved in Cholesterol Biosynthesis

The cholesterol biosynthesis pathway has been well-known for decades (See, Rudney and Sexton (1986) Regulation of Cholesterol Biosynthesis Ann. Rev. Nutrition 6:245) Coordinated regulation of cholesterol biosynthesis mRNA expression is mediated by the sterol regulatory element-binding protein (SREBP) transcription factors. Interventions that limit expression of SREBP-dependent mRNAs result in beneficial effects on circulating cholesterol.

SREBP-1a/-1c and SREBP-2 are subject to complex post-transcriptional regulation, and require a protein called SCAP for maturation into active transcription factors. In one study, genetic reduction of SCAP in mouse liver resulted in diminished SREBP protein levels, SREBP mRNA, and cholesterol biosynthesis mRNA expression (Matsuda et al., (2001) Genes Dev 15: 1206-1216). Consequently, liver cholesterol content fell ˜20%, and total plasma cholesterol fell ˜24%. A global microarray study in mouse liver confirmed that SREBPs are both necessary and sufficient for coordinated regulation of the complete suite of cholesterol biosynthesis genes (Horton et al., (2003) PNAS 100: 12027-12032).

In another study, coordinated gene repression of mouse liver sterol synthesis genes by systemic administration of a microRNA-122 antagonist also resulted in a ˜44% reduction in plasma cholesterol (Krutzfeldt et al., Nature 438 (2005): 685-689). Similar results were obtained in rodents fed a high-fat diet (Esau et al., Cell Metab 3 (2006): 87-98), and in non-human primates (Lanford et al., Science 327 (2010): 198-201). Thus, coordinated repression of cholesterol biosynthesis mRNAs results in beneficial effects on circulating cholesteroL

A number of PHI compounds were evaluated for their effect on the expression of cholesterol biosynthesis genes in mouse liver. Male Swiss Webster mice (˜25 g) were dosed orally with Compound A (90 mg/kg) or vehicle control, then sacrificed 4 h, 8 h, or 24 h later. Liver tissue was harvested and stored in RNALater (Life Technologies). Total RNA was isolated with Trizol (Life Technologies) and purified with RNEasy96 (Qiagen) according to the manufacturers' protocols. RNA concentration was measured by NanoDrop (Thermo), and quality was assessed by Bioanalyzer (Agilent).

Biotinylated microarray probe was prepared using the 3′IVT Express kit (Affymetrix) and hybridized to mouse 430A 2.0 arrays as recommended in the manufacturer's protocol. Arrays were stained, washed, and scanned using Affymetrix equipment and software. Three arrays were run for each treatment and time point, each representing the liver sample from one individual animal.

Male Swiss Webster mice (˜25 g) were dosed orally with a compound as indicated in Tables 4A and 4B (Compounds F, G, H, I, J, C, or K) or vehicle control. Animals were sacrificed 4 h or 8 h later. Each compound was tested in a separate animal study with separate controls. Liver tissue was harvested and stored in RNALater (Life Technologies). Total RNA was isolated with Trizol (Life Technologies) and purified with RNEasy96 (Qiagen) according to the manufacturers' protocols. RNA concentration was measured by NanoDrop (Thermo), and quality was assessed by Bioanalyzer (Agilent).

For microarray analysis, RNA from animals in each treatment group was pooled in pairs (n=3 pairs for 4 h vehicle, n=2 pairs for all other treatment groups, for a total of 13 arrays per compound study). This approach was used to improve the signal-to-noise ratio on microarrays. Biotinylated probe was prepared using the One-Cycle cDNA synthesis kit (Affymetrix) and hybridized to mouse 430A 2.0 arrays as recommended in the manufacturer's protocol. Arrays were stained, washed, and scanned using Affymetrix equipment and software.

Array quality was assessed with GeneChip Operating System and/or Command Console software (Affymetrix). CEL files were uploaded to GeneSpring 7.3.1 (Agilent) using GC-RMA baseline correction. Principal Components Analysis was used to identify and reject outlier arrays. Data are reported as fold-change compared to timepoint-matched vehicle controls from the same study. Values less than 1.0 indicate a reduction of RNA in the treated animal liver tissue. The RNA expression values for 16 genes in the cholesterol biosynthesis pathway are shown in Tables 4A and 4B.

TABLE 4A
Effect of Various PHI compounds on Expression of Cholesterol Biosynthesis
Genes in the Mouse Liver

| | Cpd F | | Cpd G | | Cpd H | | Cpd I | |
|---|---|---|---|---|---|---|---|---|
| | 10 mg/kg | | 10 mg/kg | | 60 mg/kg | | 10 mg/kg | |
| Gene | 4 h | 8 h | 4 h | 8 h | 4 h | 8 h | 4 h | 8 h |
| acetyl-Coenzyme A | 0.77 | 1.09 | 1.18 | 0.67 | 0.71 | 0.44 | 1.21 | 0.43 |
| acetyltransferase 2 (Acat2) | | | | | | | | |
| cytochrome P450, family 51 | 0.62 | 1.05 | 1.00 | 0.62 | 0.66 | 0.28 | 0.81 | 0.12 |
| (Cyp51) | | | | | | | | |
| 7-dehydrocholesterol reductase | 0.63 | 1.18 | 0.91 | 1.07 | 0.83 | 0.39 | 1.41 | 0.30 |
| (Dhcr7) | | | | | | | | |
| farnesyl diphosphate farnesyl | 0.56 | 1.42 | 1.15 | 0.98 | 0.59 | 0.33 | 0.85 | 0.32 |
| transferase 1 (Fdft1) | | | | | | | | |
| farnesyl diphosphate synthetase | 0.43 | 1.15 | 1.16 | 0.73 | 0.69 | 0.24 | 1.45 | 0.16 |
| (Fdps) | | | | | | | | |
| 3-hydroxy-3-methylglutaryl- | 0.17 | 0.66 | 0.68 | 0.66 | 0.23 | 0.17 | 0.41 | 0.17 |
| Coenzyme A reductase | | | | | | | | |
| (Hmgcr) | | | | | | | | |
| 3-hydroxy-3-methylglutaryl- | 0.36 | 1.23 | 1.29 | 0.82 | 0.62 | 0.19 | 1.43 | 0.19 |
| Coenzyme A synthase I | | | | | | | | |
| (Hmgcs1) | | | | | | | | |
| isopentenyl-diphosphate delta | 0.50 | 1.08 | 1.13 | 0.94 | 0.63 | 0.28 | 1.40 | 0.12 |
| isomerase (IdiI) | | | | | | | | |
| lanosterol synthase (Lss) | 0.51 | 1.04 | 1.22 | 0.87 | 0.65 | 0.35 | 0.74 | 0.28 |
| mevalonate (diphospho) | 0.28 | 0.58 | 1.03 | 1.03 | 0.45 | 0.24 | 0.45 | 0.19 |
| decarboxylase (Mvd) | | | | | | | | |
| mevalonate kinase (Mvk) | 0.76 | 0.88 | 0.98 | 0.89 | 0.74 | 0.37 | 1.02 | 0.37 |
| NAD(P) dependent steroid | 0.55 | 1.40 | 1.46 | 1.00 | 0.76 | 0.34 | 1.66 | 0.21 |
| dehydrogenase-like (Nsdhl) | | | | | | | | |
| phosphomevalonate kinase | 0.66 | 1.53 | 0.95 | 0.70 | 0.71 | 0.5 | 1.25 | 0.41 |
| (Pmvk) | | | | | | | | |
| sterol-C4-methyl oxidase-like | 0.39 | 0.92 | 0.87 | 0.58 | 0.50 | 0.19 | 0.88 | 0.11 |
| (Sc4mol) | | | | | | | | |
| sterol-C5-desaturase (fungal | 0.49 | 0.52 | 0.70 | 0.48 | 0.92 | 0.61 | 0.87 | 0.54 |
| ERG3, delta-5-desaturase) | | | | | | | | |
| homolog (S. cerevisae) (Sc5d) | | | | | | | | |
| squalene epoxidase (Sqle) | 0.48 | 0.81 | 1.50 | 1.22 | 0.95 | 0.30 | 1.36 | 0.16 |

TABLE 4B
Effect of Various PHI compounds on Expression of Cholesterol
Biosynthesis Genes in the Mouse Liver

| | Cpd J | | Cpd C | | Cpd K | | Cpd A | |
|---|---|---|---|---|---|---|---|---|
| | 10 mg/kg | | 5 mg/kg | | 10 mg/kg | | 90 mg/kg | |
| Gene | 4 h | 8 h | 4 h | 8 h | 4 h | 8 h | 4 h | 8 h |
| acetyl-Coenzyme A | 0.58 | 0.91 | 0.63 | 0.48 | 0.94 | 0.63 | 1.24 | 0.50 |
| acetyltransferase 2 (Acat2) | | | | | | | | |
| cytochrome P450, family 51 | 0.38 | 0.80 | 0.44 | 0.31 | 0.81 | 0.32 | 0.69 | 0.45 |
| (Cyp51) | | | | | | | | |
| 7-dehydrocholesterol reductase | 0.52 | 0.94 | 0.75 | 0.47 | 1.13 | 0.61 | 1.03 | 0.61 |
| (Dhcr7) | | | | | | | | |
| farnesyl diphosphate farnesyl | 0.70 | 0.66 | 0.42 | 0.50 | 0.78 | 0.39 | 0.68 | 0.43 |
| transferase 1 (Fdft1) | | | | | | | | |
| farnesyl diphosphate synthetase | 0.38 | 0.66 | 0.61 | 0.38 | 1.01 | 0.37 | 0.87 | 0.36 |
| (Fdps) | | | | | | | | |
| 3-hydroxy-3-methylglutaryl- | 0.38 | 0.43 | 0.39 | 0.41 | 0.40 | 0.11 | 0.45 | 0.43 |
| Coenzyme A reductase | | | | | | | | |
| (Hmgcr) | | | | | | | | |
| 3-hydroxy-3-methylglutaryl- | 0.28 | 0.92 | 0.29 | 0.25 | 0.74 | 0.30 | 0.94 | 0.38 |
| Coenzyme A synthase 1 | | | | | | | | |
| (Hmgcs1) | | | | | | | | |
| isopentenyl-diphosphate delta | 0.25 | 0.70 | 0.56 | 0.32 | 0.88 | 0.34 | 0.73 | 0.51 |
| isomerase (Idi1) | | | | | | | | |
| lanosterol synthase (Lss) | 0.77 | 0.76 | 0.53 | 0.45 | 0.88 | 0.38 | 0.74 | 0.44 |
| mevalonate (diphospho) | 0.72 | 0.83 | 0.41 | 0.22 | 0.68 | 0.20 | 0.68 | 0.37 |
| decarboxylase (Mvd) | | | | | | | | |
| mevalonate kinase (Mvk) | 0.79 | 0.79 | 0.53 | 0.46 | 0.92 | 0.49 | 1.00 | 0.53 |
| NAD(P) dependent steroid | 0.42 | 1.03 | 0.66 | 0.37 | 1.05 | 0.56 | 0.86 | 0.62 |
| dehydrogenase-like (Nsdhl) | | | | | | | | |
| phosphomevalonate kinase | 0.59 | 0.84 | 0.61 | 0.54 | 0.88 | 0.96 | 1.27 | 0.63 |
| (Pmvk) | | | | | | | | |
| sterol-C4-methyl oxidase-like | 0.27 | 0.52 | 0.40 | 0.40 | 0.65 | 0.21 | 0.63 | 0.46 |
| (Sc4mol) | | | | | | | | |
| sterol-C5-desaturase (fungal | 1.22 | 0.81 | 0.45 | 0.42 | 0.92 | 0.24 | 1.09 | 0.38 |
| ERG3, delta-5-desaturase) | | | | | | | | |
| homolog (S. cerevisae) (Sc5d) | | | | | | | | |
| squalene epoxidase (Sqle) | 0.26 | 1.03 | 0.60 | 0.29 | 1.46 | 0.39 | 0.61 | 0.43 |

RNA expression for all of the genes appeared to be coordinately repressed between 4 and 8 hours after administration of the compound. This effect of the HIF prolyl hydroxylase inhibitors on cholesterol biosynthetic genes could account for the lower blood cholesterol and lower LDL-C observed in subjects treated with compounds that inhibit HIF hydroxylase activity.

The effect of the HIF prolyl hydroxylase inhibitor compounds on the expression of other genes involved in cholesterol synthesis, regulation, transport and utilization were also evaluated (data not shown). The expression of the sterol regulatory element-binding protein (SREBP) transcription factors, SREBP-1 and SREBP-2, also appeared to be down-regulated by the PHI compounds. Interestingly, SREBP-1 has recently been shown to be regulated in the liver by A2b adenosine receptor (Koupenova et al. (2012) Circulation 125:354). A2b AR is a known HIF target gene. Without being held to any particular mechanism, the effect of the PHI compounds on cholesterol may be effected, at least in part, through HIF stabilization effects on A2b AR, which in turn regulates SREBPs, which mediate the coordinated regulation of the cholesterol biosynthesis genes.

### Example 7

### Effect of Compound a on Total Cholesterol and LDL-C Levels in CKD Patients

Compound A used in a phase 2b study to test the efficacy for correction of anemia in subjects with chronic kidney disease (CKD). Subjects with CKD and hemoglobin (Hb) less than 10 g/dL were randomized 2:1 to Compound A or placebo administered orally three times a week for 8 weeks. Two cohorts tested different starting doses Compound A, with dose escalation permitted at Week 5 if Hb change in the first 4 weeks was <1 g/dL. The Hb target was 11 g/dL. The primary efficacy endpoint was the maximum change of Hb from baseline by Week 9. The percentage of Compound A-treated subjects who achieved Hb response (Hb increase of >1 g/dL from baseline Hb) (86.4%) was significantly higher than the percentage of placebo-treated subjects who achieved Hb response (24.2%). Significant decreases in total cholesterol and LDL-C were observed in the Compound A treated subjects compared to the placebo-treated subjects. Although HDL-C also decreased in the Compound A treated subjects, these subjects exhibited an improved HDL/LDL ratio. The results are shown in Table 5.

TABLE 5

| | Compound A | Placebo |
|---|---|---|
| Number of Subjects | 6.1 | 30 |
| Mean BL total cholesterol | 166.84 mg/dL | 182.60 mg/dL |
| Mean BL LDL-C | 102.60 mg/dL | 114.76 mg/dL |
| Cholesterol total change, | −33.9 (−18.5%)*** | +8 (+5.5%) |
| mg/dL (% change) | | |
| LDL Cholesterol change | −27.9 (−23.2%)*** | +4 |
| mg/dL, (% change) | | |
| HDL/LDL ratio change | 0.097** | +0.008 |

**p < 0.01
***p < 0.0001

Subjects were stratified by baseline (BL) total cholesterol (either >200 mg/dL or s 200 mg/dL) and analyzed for mean total cholesterol and LDL-C over the course of treatment. Compound A-treated subjects (n=48 for BL≦200 mg/dL; n=13 for BL>200 mg/dL) showed reduction in total cholesterol (FIG. 14) and in LDL-C (FIG. 15). Placebo treated subjects (n=20 for BL≦≦200 mg/dL; n=10 for BL>200 mg/dL) showed no significant change in total cholesterol (FIG. 14) or LDL-C (FIG. 15).

### Example 8

### Effect of Compound a on Total Cholesterol and LDL-C Levels in ESRD Patients

This study was designed to test the ability of Compound A to replace epoetin alfa for treatment of anemia in subjects with end stage renal disease (ESRD) who were on stable doses of epoetin alfa and had hemoglobin (Hb) levels of between 9.0 and 12.0 g/dL. Three cohorts of 28 subjects each were randomized (3:1) to Compound A orally three times a week for six weeks or to continue epoetin alfa. Three dose levels of Compound A were tested (mean dose in u/kg/week for the three cohorts was 4.15 mg, 4.78 mg, and 5.82 mg). The primary endpoint was maintaining Hb no lower than 0.5 g/dL below baseline. 893% of subjects over all three Compound A-treated cohorts achieved the primary endpoint with respect to Hb at the end of treatment. In addition, subjects randomized to Compound A had significantly lower total cholesterol and LDL cholesterol levels compared to subjects randomized to epoetin alfa, as shown in Table 6. Mean total cholesterol change for the Compound A-treated subjects (all cohorts) was a decrease of 15 mg/dL compared to an increase of 18 mg/dL for the epoetin alfa treated subjects. Mean change in LDL-C for Compound A-treated subjects (all cohorts) was a decrease of 25 mg/dL compared to a mean increase of 5 mg/dL in LDL-C for epoetin alfa treated subjects. HDL-C showed a decrease in the Compound A treated subjects, HDL-C in epoetin alfa treated subjects did not change significantly. Subjects were stratified by baseline (BL) total cholesterol (either >200 mg/dL or <200 mg/dL) and analyzed for mean total cholesterol and LDL-C over the course of treatment. Both groups of Compound A treated subjects (n=60 for BL ≦200 mg/dL; n=14 for BL ≧200 mg/dL) showed reduction in total cholesterol (FIG. 16) and in LDL-C (FIG. 17). Both groups of epoetin treated subjects (n=20 for BL ≦200 mg/dL; n=2 for BL >200 mg/dL) showed an increase in total cholesterol (FIG. 16). The epoetin treated subjects with the higher BL total cholesterol (n=2) showed a slight decrease in LDL-C (FIG. 17), those with the lower BL total cholesterol (n=20) showed an increase in LDL-C

TABLE 6

| | Total Compound A, | Epoetin alfa, |
|---|---|---|
| | Mean (±SD) | Mean (±SD) |
| | (3 cohorts, n = 74) | (n = 22) |
| Dose (u/kg/week, Mean) | 4.92 mg | 149.6 U |
| Mean BL Total Cholesterol (mg/dL) | 171.11 | 158.41 |
| Mean BL LDL Cholesterol (mg/dL) | 101.71 | 90.75 |
| Total Cholesterol change (mg/dL) | −15 (38)** | +18 (22) |
| LDL cholesterol change (mg/dL) | −25 (22)** | +5 (14) |

**p < 0.05 compared to epoetin alfa

### Example 9

### Effect of Compound C on Total Cholesterol and LDL-C in Healthy Volunteers

Compound C was administered weekly for 4 weeks (at days 1, 8, 15, and 22) to healthy human subjects at either 0.15 mg/kg (n=7) or 0.25 mg/kg (n=6). Placebo was administered in the same regimen as a control (n=2 or n=1). Fasting blood samples for cholesterol analysis were collected from subjects in the morning on the day of dosing before compound or placebo was administered. At both dosing levels of Compound C, total cholesterol levels and LDL-C levels decreased over the course of treatment. FIGS. 18 and 19 show the % change from baseline for total cholesterol and LDL-C, respectively, for Compound C at 0.15 mg/kg compared to placebo. FIGS. 20 and 21 show the change from baseline for total cholesterol and LDL-C, respectively, for Compound C at 0.25 mg/kg compared to placebo.

### Example 10

### Mouse High Cholesterol Models

Ten-week-old male apoE knockout mice (Jackson Laboratories, Bar Harbor, Me.) were fed a normal chow diet (0.02% cholesterol, 13.5% fat by weight; LabDiet). Ten-week-old male diet induced obese mice (DIO Mice, Jackson Laboratories, Bar Harbor, Me.) were fed a high fat diet (0.095% cholesterol, 60% fat by weight; ResearchDiets) starting at six-weeks-old. After 2 weeks of acclimatization, whole blood was collected for baseline measurement before administration of test reagents. All test reagents were administered orally by gavage needle three times a week for 2 weeks for the apoE mice, and three times a week for 4 weeks for the DIO mice. The animals were sacrificed at end of study. Whole blood was collected weekly in Heparin-tube by submandibular bleeding and isolated plasma was stored at −80° C. for measurement of total cholesterol.

### Example 11

### Effect of PHI compounds in a High Cholesterol Mouse Model—ApoE Deficient Mice

ApoE deficient mice as described in Example 10 were used to test the effect of various compounds on cholesterol levels. Compound (60 mg/kg) or vehicle was administered to the mice (n=10/group) three times a week for two weeks. A statin compound (rosuvastatin) was also tested. Blood samples were collected at the end of the study and total cholesterol blood levels were tested. A group of 10 mice was sacrificed at the beginning of the study for a baseline total cholesterol measurement. Total cholesterol levels at the end of the study for mice treated with Compound B, Compound E, Compound D, Compound F, Compound G, and rosuvastatin are shown in FIG. 22, compared to the baseline total cholesterol (baseline) and control mice treated with vehicle. Compound D significantly decreased cholesterol levels in the treated mice compared to baseline and Compound B showed a trend towards lower total cholesterol compared to baseline. The other compounds, including rosuvastatin, did not show a reduction in cholesterol in this model. Typically statins show mixed results in this model and are not very effective to reduce cholesterol in mice (Bea et al. 2003 Atherosclerosis 167:187-194).

### Example 12

### Effect of PHI compounds in a High Cholesterol Mouse Model—Diet Induced Obesity (DIO)

DIO mice as described in Example 10 were used to test the effect of various compounds on cholesterol levels. Compound (60 mg/kg) or vehicle was administered to the mice (n=10/group) three times a week for four weeks. A statin compound (rosuvastatin) was also tested. Blood samples were collected at weekly and total cholesterol blood levels were tested. Percent change from baseline in total cholesterol levels at two weeks for mice treated with Compound B, Compound E, Compound D, Compound F, Compound G, or rouvastatin are shown in FIG. 23 compared to the control mice that were treated only with vehicle. The vehicle treated mice exhibited an increase in mean total cholesterol at week 2 of about 70% compared to baseline. All tested compounds in this study showed lower % change in total cholesterol after two weeks of dosing compared to the vehicle treated. The mice treated with compound F or G in this study had lower body weights at the end of the study which might indicate some toxicity of these compounds at the doses used. Rosuvastatin-treated mice also exhibited lower total cholesterol levels in this model.

In a another study with DIO mice, Compound A (60 mg/kg), Compound L (20 mg/kg), Compound M (20 mg/kg), Compound N (20 mg/kg), rosuvastatin (20 mg/kg) or vehicle were administered to the mice (n=10/group) three times a week for two weeks. Compound A-treated mice showed a statistically significant decrease in total cholesterol levels compared to vehicle treated. These results are shown in FIG. 24.

### Example 13

### Cholesterol Reduction and Erythrogenesis

To test whether the reduction in cholesterol level by treatment with a prolyl hydroxylase inhibitor is related to the erythrogenesis or increase in hemoglobin levels that is associated with treatment with PHI, ApoE deficient mice (n=8/group) were administered Compound A or Compound D orally at 2, 20, 60, or 100 mg/kg, three times a week for 4 weeks. Total cholesterol and hemoglobin were measured weekly. At these doses, Compound A increased hemoglobin in a dose dependent manner exhibiting statistically significant increases in hemoglobin compared to vehicle-treated at the 60 mg/kg and 100 mg/kg doses (FIG. 25A). Compound D did not significantly increase hemoglobin at any of the tested doses (FIG. 25C). Both Compound A-treated and Compound D-treated animals showed lower total cholesterol levels (as shown by % change from baseline in FIGS. 25B and 25D) compared to vehicle-treated at all tested doses, with statistically significant decreases in total cholesterol (% change) at 100 mg/kg dose for Compound A (FIG. 25B), and at 20, 60, and 100 mg/kg doses for Compound D (FIG. 25D). These results suggest that the reduction in total cholesterol is not correlated with the increase in hemoglobin.

### Example 14

### Effect of PHI Compounds on Expression of Genes Involved in Cholesterol Biosynthesis in DIO Mice

Liver tissue was collected as described in Example 6 from DIO mice at the end of the study (4 weeks of dosing) described in Example 12. RNA isolation and microarray analysis were carried out as described in Example 6. The expression of 16 genes involved in cholesterol biosynthesis in mice treated with Compound B, Compound E, or Compound D, relative to that in the vehicle treated mice, is shown in Table 7.

TABLE 7

| | Cpd | Cpd | Cpd |
|---|---|---|---|
| Gene | B | E | D |
| acetyl-Coenzyme A acetyltransferase 2 (Acat2) | 0.43 | 0.69 | 0.72 |
| cytochrome P450, family 51 (Cyp51) | 0.18 | 0.26 | 0.55 |
| 7-dehydrocholesterol reductase (Dhcr7) | 0.43 | 0.52 | 0.76 |
| farnesyl diphosphate farnesyl transferase 1 (Fdft1) | 0.30 | 0.40 | 0.44 |
| farnesyl diphosphate synthetase (Fdps) | 0.26 | 0.57 | 0.92 |
| 3-hydroxy-3-methylglutaryl-Coenzyme A reductase | 0.20 | 0.36 | 0.47 |
| (Hmgcr) | | | |
| 3-hydroxy-3-methylglutaryl-Coenzyme A synthase 1 | 0.15 | 0.33 | 0.42 |
| (Hmgcs1) | | | |
| isopentenyl-diphosphate delta isomerase (Idi1) | 0.23 | 0.42 | 0.67 |
| lanosterol synthase (Lss) | 0.38 | 0.45 | 0.70 |
| mevalonate (diphospho) decarboxylase (Mvd) | 0.20 | 0.34 | 0.43 |
| mevalonate kinase (Mvk) | 0.54 | 0.58 | 0.77 |
| NAD(P) dependent steroid dehydrogenase-like | 0.34 | 0.77 | 1.02 |
| (Nsdhl) | | | |
| phosphomevalonate kinase (Pmvk) | 0.48 | 0.74 | 1.14 |
| sterol-C4-methyl oxidase-like (Sc4mol) | 0.13 | 0.27 | 0.38 |
| sterol-C5-desaturase (fungal ERG3, delta-5- | 0.30 | 0.34 | 0.42 |
| desaturase) homolog (S. cerevisae) (Sc5d) | | | |
| squalene epoxidase (Sqle) | 0.21 | 0.52 | 0.89 |

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

1. A method of reducing the circulating level of low density lipoprotein cholesterol (LDL-C) in a subject in need thereof, the method comprising administering to the subject an effective amount of a compound that inhibits HIF hydroxylase activity, whereby the circulating level of LDL-C in said subject is reduced.

2. (canceled)

3. (canceled)

4. A method of treating high cholesterol in a subject in need thereof, the method comprising reducing the circulating level of low density lipoprotein cholesterol (LDL-C) in a subject having high cholesterol by administering to the subject an effective amount of a compound that inhibits HIF hydroxylase activity, whereby the circulating level of LDL-C in said subject is reduced and the high cholesterol is treated.

5. A method of reducing the circulating level of total cholesterol in a subject under treatment for high cholesterol with a cholesterol lowering agent selected from the group consisting of a HMGCoA reductase inhibitor, a nicotinic acid, a fibric acid, and a bile acid-binding resin, the method comprising administering to the subject an effective amount of a compound that inhibits HIF hydroxylase activity, whereby the circulating level of total cholesterol is reduced.

6. The method of claim 1, wherein the compound that inhibits HIF hydroxylase activity is a heterocyclic carboxamide.

7. The method of claim 6, wherein the compound that inhibits HIF hydroxylase activity is selected from the group of a compound of Formula I, a compound of Formula II, a compound of Formula III, and a compound of Formula IV.

8. The method of claim 6, wherein the compound is an isoquinoline carboxamide.

9. (canceled)

10. (canceled)

11. The method of claim 6, wherein the compound is selected from the group consisting of [(4-Hydroxy-1-methyl-7-phenoxy-isoquinoline-3-carbonyl)-amino]-acetic acid, [(1-Chloro-4-hydroxy-isoquinoline-3-carbonyl)-amino]-acetic acid, {[5-(4-Chloro-phenoxy)-1-cyano-4-hydroxy-isoquinoline-3-carbonyl]-amino}-acetic acid, {[4-Hydroxy-7-(4-methoxy-phenoxy)-isoquinoline-3-carbonyl]-amino}-acetic acid, [(1-Cyano-4-hydroxy-5-phenoxy-isoquinoline-3-carbonyl)-amino]-acetic acid, {[2,3-Dichloro-7-cyano-4-hydroxy-1-(4-methoxy-benzyl)-1H-pyrrolo[2,3-c]pyridine-5-carbonyl]-amino}-acetic acid, {[7-Cyano-1-(2-fluoro-benzyl)-4-hydroxy-1H-pyrrolo[2,3-c]pyridine-5-carbonyl]-amino}-acetic acid, [(1-Cyano-4-hydroxy-7-isopropoxy-isoquinoline-3-carbonyl)-amino]-acetic acid, {[1-Cyano-7-(2,6-dimethyl-phenoxy)-4-hydroxy-isoquinoline-3-carbonyl]-amino}-acetic acid, [(7-Cyano-4-hydroxy-1-naphthalen-2-ylmethyl-1H-pyrrolo[2,3-c]pyridine-5-carbonyl)-amino]-acetic acid, and {[3-Bromo-7-cyano-4-hydroxy-1-(4-methoxy-benzyl)-1H-pyrrolo[2,3-c]pyridine-5-carbonyl]-amino}-acetic acid.

12. The method of claim 1, wherein the subject is human.

13. The method of claim 12, wherein the subject has a high circulating level of total cholesterol.

14. The method of claim 12, wherein the subject has a circulating level of total cholesterol of 200 mg/dL or greater.

15. (canceled)

16. The method of claim 12, wherein the subject has a high circulating level of LDL-C.

17. The method of claim 12, wherein the subject has a circulating level of LDL-C of greater than 100 mg/dL.

18. (canceled)

19. (canceled)

20. The method of claim 12, wherein the subject has chronic kidney disease.

21. The method of claim 12, wherein the subject end stage renal disease.

22. The method of claim 12, wherein the subject is anemic.

23. The method of claim 1, wherein the circulating LDL-C level is reduced from the pre-treatment level by at least 10%.

24. (canceled)

25. (canceled)

26. The method of claim 1, wherein the circulating LDL-C level is reduced from the pre-treatment level by at least 10 mg/dL.

27. (canceled)

28. (canceled)

29. (canceled)
